(19) United States
(12) Patent Application Publication
(10) Pub. No.: US 2024/0117357 A1
(43) Pub. Date: Apr. 11, 2024

(54) REGULATION OF METABOLISM AND OBESITY BY MITOCHONDRIAL MUL1 E3 UBIQUITIN LIGASE

(71) Applicant: University of Central Florida Research Foundation, Inc., Orlando, FL (US)

(72) Inventors: Antonis S. Zervos, Orlando, FL (US); Lucia Cilenti, Orlando, FL (US)

(73) Assignee: University of Central Florida Research Foundation, Inc., Orlando, FL (US)

(21) Appl. No.: 18/328,381

(22) Filed: Jun. 2, 2023

Related U.S. Application Data
(60) Provisional application No. 63/348,687, filed on Jun. 3, 2022.

(51) Int. Cl. C12N 15/113; A61K 31/201; A61P 3/04; C12N 9/22; G01N 1/30; G01N 33/50; G01N 33/92

(52) U.S. Cl. CPC C12N 15/1137; A61K 31/201; A61P 3/04; C12N 9/22; G01N 1/30; G01N 33/5067; G01N 33/92; C12N 2310/20

(57) ABSTRACT

A novel molecular pathway involving the MUL1 E3 ubiquitin ligase that regulates mitochondrial metabolism including regulation of lipogenesis and adiposity has been discovered. The significance of this pathway is exemplified in animals in which Mul1 is inactivated. These animals have a metabolic phenotype, and they are resistant to high fat diet (HFD)-induced obesity. Applications include an inactivator of mitochondrial Mul1 E3 ubiquitin ligase, compositions/kits including this inactivator, and methods for using this inactivator to regulate/inactivate mitochondrial Mul1 E3 ubiquitin ligase for prevention and/or treatment of conditions such as weight gain, obesity, type 2 diabetes, and nonalcoholic fatty liver disease (NAFLD).

### STATEMENT REGARDING FEDERALLY SPONSORED RESEARCH OR DEVELOPMENT

This invention was made with government support under HL132511 awarded by the National Institutes of Health. The U.S. government has certain rights in the invention.

### REFERENCE TO AN ELECTRONIC SEQUENCE LISTING

The content of the electronic sequence listing (File Name: UCF010SEQLIST.xml; Size: 2,886 bytes; and Date of Creation: Jun. 2, 2023) is herein incorporated by reference in its entirety.

### FIELD OF THE INVENTION

The invention generally relates to analysis of the molecular pathways regulating metabolic states of cells; particularly to a novel molecular pathway that involves the MUL1 E3 ubiquitin ligase (MUL1) that regulates mitochondrial metabolism and fat oxidation; and most particularly to inhibition of MUL1 in this new pathway as a target for development of pharmacological inhibitors of MUL1 for treatment of obesity and associated metabolic syndromes.

### BACKGROUND

Mitochondria are responsible for energy production as well as the synthesis of intermediates used in macromolecular assembly. Accordingly, mitochondria can alter their bioenergetic and biosynthetic function to meet the metabolic demands of the cell or in response to altered physiological environment such as low O₂ levels (hypoxia) or nutrient stress. The regulation of metabolism necessitates continuous and effective communication between the mitochondria with the rest of the cell which involves numerous proteins with diverse functions.

Mitochondria function to meet the high energy demand when cells are subjected to various conditions by providing ATP through oxidative phosphorylation (OXPHOS). Low oxygen levels activate signaling pathways that provide metabolic and adaptive mechanisms to the new environment (1). HIF-1α is the primary transcriptional regulator of the cell response to low oxygen levels (hypoxia) (2-4). Accumulation of HIF-1α protein and its translocation to the cell nucleus leads to the transcriptional activation of several hundred genes that carry a hypoxia response element (HRE) in their promoters (5, 6). This leads to HIF-la-dependent reprogramming of cellular metabolism that shifts the ATP production from oxidative phosphorylation, which is limited under low oxygen levels, to glycolysis (7, 8). There is an important phenomenon (Warburg effect) associated with most cancer cells where glycolysis is predominantly used as the main source of ATP production, even under normal levels of oxygen (normoxia) (9, 10). Accumulating evidence indicates that induced aerobicglycolysis is also important in many cellular processes including embryogenesis, innate and adaptive immunity, type 2 diabetes, starvation, as well as cardiomyopathy (9-15). The role of aerobic glycolysis in these processes is unclear but it provides a swift supply of ATP as well as the building blocks to support many cellular processes and cell growth (16-18). The mechanism that potentially “bypasses” the tight regulation of cellular metabolism by the HIF-1α aerobic conditions is also unknown. Under normoxia (aerobic conditions), HIF-1α is continuously synthesized and degraded in the cytosol through a highly regulated process that involves the CRL2^{VHL} ligase complex and proteasomal degradation (19, 20). The oxygen sensor propyl hydroxylase 2 (PHD2) hydroxylates HIF-1α which then binds to the Von Hippel-Lindau (VHL) tumor suppressor protein and gets ubiquitinated by the CRL2^{VHL} ligase complex (21).

MUL1 is a multifunctional E3 ubiquitin ligase anchored in the outer mitochondrial membrane (OMM) with its RING finger domain facing the cytoplasm. FIGS. 1A-B. MUL1 participates in various biological pathways involved in mitophagy, apoptosis, mitochondrial dynamics, and innate immune response. The unique topology of MUL1 enables it to “sense” mitochondrial stress in the intermembrane mitochondrial space and convey these signals through the ubiquitination of specific cytoplasmic substrates. FIG. 2A. MUL1 can perform K48- and K63-ubiquitination, as well as SUMOylation FIG. 2B. The instant inventors' study focuses on MUL1's specific K48-ubiquitination of four MUL1 specific substrates, UBXN7, ULK1, Mfn2, and Akt2, that accumulate in MUL1(−/−) cells, which exhibit altered mitochondrial metabolism.

The instant inventors have identified a novel pathway that operates upstream of the CRL2^{VHL} ligase complex. This pathway originates in the mitochondria and involves the regulation of the UBXN7 cofactor by the mitochondrial MUL1 ligase. MUL1 ubiquitinated UBXN7 and targets it for degradation by the proteosome. FIGS. 3A-C. This allows a tight regulation of UBXN7 protein level that is kept low but sufficient for the normal function of the CRL2^{VHL} ligase complex. If UBXN7 is deregulated, as happens when MUL1 is inactive, the CRL2^{VHL} ligase complex becomes inoperable leading to accumulation of UBXN7 and HIF-1α, reduction in oxidative phosphorylation, and a metabolic shift from OXPHOS to glycolysis under aerobic conditions. Thus, Mul1, through UBXN7, supports a reciprocal regulation of HIF-1α and NRF2 proteins.

It is becoming abundantly clear that aerobic glycolysis is paramount not only in cancer but also in many biological processes involving proliferating cells as it provides a swift supply of ATP as well as the building blocks to support many cellular processes and cell growth (18). The instant inventors are the first to show this new pathway wherein mitochondria, through MUL1, regulate HIF-1α protein levels. The data presented herein show this pathway is very important under normal conditions and is potentially involved in mitochondrial induction of aerobic glycolysis. Considering its involvement in this pathway, MUL1 is a promising target for drug development.

### SUMMARY OF THE INVENTION

Modulation of MUL1 reveals promising therapeutic options for treatment of various conditions such as obesity and its associated conditions and metabolic syndromes. As discussed, MUL1 is a multifunctional E3 ubiquitin ligase that is involved in various pathophysiological processes including apoptosis, mitophagy, mitochondrial dynamics, and innate immune response. The studies disclosed herein revealed a new function for MUL1 in the regulation of mitochondrial metabolism. The metabolic phenotype of MUL1(−/−) cells was characterized using metabolomic, lipidomic, gene expression profiling, metabolic flux, and mitochondrial respiration analyses. In addition, the mechanism by which MUL1 regulates metabolism was investigated. The transcription factor HIF-1α, as well as the serine/threonine kinase Akt2, were identified as the mediators of the MUL1 function. MUL1 ligase, through K48-specific polyubiquitination, regulates both Akt2 and HIF-1α protein level and the absence of MUL1 leads to the accumulation and activation of both substrates. Specific chemical inhibitors, and activators of HIF-1α and Akt2 proteins, as well as Akt2(−/−) cells, were used to investigate the individual contribution of HIF-1α and Akt2 proteins to the MUL1-specific phenotype. This study describes a new function of MUL1 in the regulation of mitochondrial metabolism and reveals how its downregulation/inactivation can affect mitochondrial respiration and cause a shift to a new metabolic and lipidomic state. The significance of this pathway is exemplified in animals where MUL1 is inactivated. These animals have a metabolic phenotype, and they are resistant to high fat diet (HFD)-induced obesity.

In a most general aspect, the invention provides a new paradigm for treatment of obesity. In a general aspect, the invention reveals a new function of MUL1 E3 ubiquitin ligase in the regulation of mitochondrial metabolism. MUL1 E3 ubiquitin ligase is also referred to herein as “Mul1”, “MUL1”, “MUL1” and “Mul1.” In a general aspect, the invention reveals a new function of MUL1 E3 ubiquitin ligase in the regulation of lipogenesis and adiposity. In another general aspect, the invention provides a new MUL1 molecular pathway for targeting in drug development. In another general aspect, the invention provides inactivators of MUL1. In another general aspect, the invention provides downregulators of the expression of MUL1. In another general aspect, the invention provides pharmacological modulators of MUL1. In yet another general aspect, the invention provides pharmacological regulators of MUL1. In another general aspect, the invention provides pharmacological inhibitors of MUL1.

In an aspect, the invention provides a modulator of mitochondrial MUL1 E3 ubiquitin ligase. The term “modulator” makes non-limiting reference to a substance and/or composition capable of affecting expression or function of MUL1.

In another aspect, the invention provides a regulator of mitochondrial MUL1 E3 ubiquitin ligase. The term “regulator” makes non-limiting reference to a substance and/or composition capable of controlling expression or function of MUL1. A regulator may inhibit or inactivate MUL1. A non-limiting example of a regulator of mitochondrial Mul1 E3 ubiquitin ligase is an inhibitor that inhibits and/or prevents expression or function of MUL1.

In an aspect, the invention provides a pharmaceutical composition including a modulator or a regulator of mitochondrial MUL1 E3 ubiquitin ligase and at least one pharmaceutically acceptable carrier. The phrase “pharmaceutically acceptable carrier” refers to an inactive and non-toxic substance used in association with an active substance, i.e. a pharmacological inhibitor of MUL1, especially for aiding in the application/delivery of the active substance. “Inactive”, in this context, refers to inactivity with regard to the activity of the active substance. Non-limiting examples of pharmaceutically acceptable carriers are diluents, fillers, binders, disintegrants, superdisintegrants, flavorings, sweeteners, lubricants, alkalizers/alkalinizing agents, and absorption enhancers/penetration enhancers/permeation enhancers. Pharmaceutically acceptable carriers can be in any usable form such as a solid or a liquid. Further, pharmaceutically acceptable carriers can have more than one function, a non-limiting e.g., a filler can also be a disintegrant. Additionally, pharmaceutically acceptable carriers may also be referred to as non-medicinal ingredients (NMIs) or pharmaceutically acceptable excipients. Any pharmaceutically acceptable carrier used for production and/or delivery of drugs is contemplated for use with the inventive modulator/regulator/inhibitor.

Another aspect of the invention includes a method for inhibiting and/or inactivating mitochondrial Mul1 E3 ubiquitin ligase in a cell. The method includes providing a pharmaceutical composition, including a modulator or a regulator of mitochondrial MUL1 E3 ubiquitin ligase and at least one pharmaceutically acceptable carrier, to the cell and administering the pharmaceutical composition to the cell.

Another aspect of the invention includes a method for inhibiting and/or inactivating mitochondrial Mul1 E3 ubiquitin ligase in a subject in need thereof. The method includes providing a pharmaceutically effective amount of a pharmaceutical composition, including a modulator or a regulator of mitochondrial MUL1 E3 ubiquitin ligase and at least one pharmaceutically acceptable carrier, to the subject and administering the pharmaceutical composition to the subject.

The term “subject” refers to any human or animal who will benefit from use of the compositions, methods, and/or treatments described herein. A preferred, but non-limiting subject is a human patient having difficulty with weight management.

The phrase “effective amount”, “pharmaceutically effective amount” or “therapeutically effective amount” refers to the amount of a composition necessary to achieve the composition's intended function, for example, in the instant invention, a decrease in weight or resistance to weight gain.

Another aspect of the invention includes a method for treating a condition responsive to regulation of mitochondrial Mul1 E3 ubiquitin ligase in a subject in need thereof. The method includes providing a pharmaceutically effective amount of a pharmaceutical composition, including a modulator or a regulator of mitochondrial MUL1 E3 ubiquitin ligase and at least one pharmaceutically acceptable carrier, to the subject and administering the pharmaceutical composition to the subject. A non-limiting example of regulation is inhibition or inactivation. The condition responsive to regulation of mitochondrial Mul1 E3 ubiquitin ligase can be, but is not limited to, weight gain, obesity, obesity-associated conditions, and metabolic syndrome. The obesity-associated condition can be, but is not limited to, non-alcoholic fatty liver disease, Type II diabetes, heart disease, kidney disease, stroke, and cancer.

Another aspect of the invention includes a method for preventing and/or reversing weight gain and/or obesity in a subject in need thereof. The method includes providing a pharmaceutically effective amount of a pharmaceutical composition, including a modulator or a regulator of mitochondrial MUL1 E3 ubiquitin ligase and at least one pharmaceutically acceptable carrier, to the subject and administering the pharmaceutical composition to the subject.

Another aspect of the invention includes a method for regulating weight gain in a subject in need thereof. The method includes providing a pharmaceutically effective amount of a pharmaceutical composition, including a modulator or a regulator of mitochondrial MUL1 E3 ubiquitin ligase and at least one pharmaceutically acceptable carrier, to the subject and administering the pharmaceutical composition to the subject.

In another aspect, the components of the inventive compositions can be packaged in containers and assembled in kits together with instructions for use.

In yet another aspect, the invention provides markers for identification or recognition of the inactivation of MUL1 such as, but not limited to a gene expression signature indicative of inactivation of mitochondrial MUL1 E3 ubiquitin ligase. The gene expression signature exhibits upregulation of trefoil factor 3 (TFF3), asparagine synthetase (ASNS), and cytochrome P450 2A4 (CYP2A4) genes, and downregulation of stearoyl-CoA desaturase 1 (SCDI) gene.

In another aspect, the invention provides a method for inhibiting lipogenesis in cells, such as, but not limited to, human cells or animal cells. A preferred example of cells is human liver cells. The method includes providing cells, inactivating mitochondrial MUL1 E3 ubiquitin ligase (MUL1) in the cells; treating a portion of the cells with a simulator of a high fat diet for a pre-determined period of time; using a remaining portion of the cells as untreated control cells; applying a stain for identification of lipid droplets to the treated cells and untreated control cells; identifying lipid droplets in the treated cells and in the untreated control cells using a stain capable of identification of lipids; visualizing lipid droplets identified in the treated cells and in the untreated control cells using an optical instrument; and comparing an amount of stained lipid droplets visualized in the treated cells to an amount of stained lipid droplets visualized in the untreated control cells, wherein a decrease of stained lipid droplets in the treated cells indicates inhibition of lipogenesis in the treated cells. CRISPR-Cas9, is a preferred, but non-limiting system for inactivating MUL1. Treating cells with oleic acid for 24 hours is a preferred, but non-limiting, example of treating a portion of the cells with a simulator of a high fat diet for a pre-determined period of time.

In yet another aspect, the invention provides a method for protecting a subject against obesity induced by a high fat diet (HFD) by inactivation of mitochondrial MUL1 E3 ubiquitin ligase (MUL1). The method includes providing an inactivator of MUL1, such as, but not limited to, an inhibitor of MUL1; and administering the inactivator of MUL1 to the subject, thereby inactivating MUL1 and protecting the subject against obesity induced by a high fat diet (HFD). A “high fat diet” refers to a diet in which at least 60% of the calories are obtained from fat. Preferred, but non-limiting subjects, are overweight human patients and their overweight companion animals.

The above-listed aspects are exemplary embodiments only and are not meant to limit the invention. Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIGS. 1A-B are drawings illustrating a mitochondrion, the cellular organelle of energy production. FIG. 1A illustrates the parts of the mitochondrion and FIG. 1B illustrates the location of Mul1 E3 ubiquitin ligase embedded in the outer mitochondrial membrane.

FIGS. 2A-B are schematic diagrams illustrating Mul1 pathways. FIG. 2A is a schematic diagram illustrating the ubiquitination pathway. FIG. 2B is a schematic diagram of Mul1 pathway and its targets. Mul1 ubiquitin ligase is involved in K63, K48 ubiquitination as well as SUMOylation of various substrates involved in diverse processes.

FIGS. 3A-C are schematic diagrams illustrating the new pathway that operates upstream of the CRL2^{VHL} complex. This pathway involves the UBXN7 cofactor protein and its regulation by mitochondrial Mul1 E3 ubiquitin ligase. FIG. 3A shows the active Mul1 E3 ubiquitin ligase and FIG. 3B shows the inactive Mul1 E3 ubiquitin ligase. FIG. 3C shows the CRL3^{KEAP1} and the CRL2^{VHL} complexes.

FIGS. 4A-I show data illustrating that MUL1 is a multifunctional E3 ubiquitin ligase and its inactivation leads to the accumulation of its various substrates. FIG. 4A is a schematic diagram illustrating activity of MUL1. MUL1 is a mitochondrial E3 ligase with a diverse role in immune response, mitochondrial dynamics/mitophagy and metabolism. MUL1 mediates its function through K63, or K48-ubiquitination, as well as SUMOylation of the specific substrates shown. FIG. 4B shows a Western blot analysis used to monitor the protein level of several known MUL1 K48 ubiquitination substrates in two independent clones of HEK293 WT and MUL1(−/−) cells. β-actin was used to verify equal loading in each lane. FIG. 4C is a bar graph representing the densitometrical analysis of the proteins shown in FIG. 4B, normalized against β-actin. Results shown as means±SD of three independent experiments. *, P≤0.02: MUL1(−/−) vs WT. FIG. 4D shows a Western blot analysis used to monitor the Akt1, Akt2, Akt3 protein level as well as phosphorylated AKT form (P-AKT Thr 308) and the GSK-3β substrate (P-GSK-3β S9) in WT and MUL1(−/−) cells. FIG. 4E is a bar graph representing the densitometrical analysis of upregulated proteins shown in FIG. 4D, normalized against β-actin. Results shown as means±SD of three independent experiments. *, P≤0.03: MUL1(−/−) vs WT.

FIGS. 4F-G show data illustrating that MUL1 inactivation in HeLa cells affects Akt2 and HIF-1α, protein levels, and metabolic flux. FIG. 4F shows a Western blot analysis. HeLa WT and HeLa MUL1 (−/−) cells were treated with 5 μM of MG132 for 4 hours; whole cell extracts were prepared; and the Western blot analysis was used to monitor UBXN7, HIF-1α, GLUT1, Akt2, P-GSK-3(3 S9, as well as MUL1 protein expression. β-actin was used to verify equal loading in each lane. FIG. 4G is a bar graph representing the densitometrical analysis of the proteins shown in FIG. 4F, normalized against β-actin. Results shown as means±SD of three independent experiments. *, P≤0.04: MUL1(−/−) con vs WT con.

FIGS. 4H-I show data illustrating the role of UBXN7 and Akt2 in Mul1 regulation of metabolism. FIG. 4H shows a Western blot analysis. Akt2 and its phosphorylated form (P-Akt2 Thr 308) accumulated in MUL1(−/−) cells, whereas there was no difference in Akt1 and Akt3 proteins. MUL1 specifically targets Akt2 kinase in its active form. FIG. 4I is a graph representing the densitometrical analysis of the protein shown in FIG. 4H, normalized against β-actin. *: p<0.004 vs WT.

FIGS. 5A-F show data illustrating the role of Akt2 and HIF-1α in mitochondrial respiration and glycolytic phenotype of HEK293 MUL1(−/−) cells. HEK293 WT and MUL1(−/−) cells were treated with either 2.5 μM perifosine (Peri), 500 nM chetomin (CTM), or both drugs for 4 hours. FIG. 5A is a graph showing monitoring of the Glycolytic capacity in WT and MUL1(−/−) cells using the Glycolytic Stress Test. Extracellular Acidification Rate (ECAR) was measured using the Seahorse Extracellular Flux analyzer. #, P≤0.04: MUL1(−/−) con vs WT con; *, P≤0.03: MUL1(−/−) treated vs MUL1(−/−) con. FIG. 5B is a bar graph showing quantification of the glycolysis, glycolytic capacity, and glycolytic reserve obtained from three independent experiments. #, P≤0.04: MUL1(−/−) con vs WT con; *, P≤0.03 for MUL1(−/−) treated vs MUL1(−/−) con. FIG. 5C is a graph showing monitoring of mitochondrial respiration in WT and MUL1(−/−) cells using the Mitochondrial Stress Test. Oxygen Consumption Rate (OCR) was measured with Seahorse analyzer. FIG. 5D is a bar graph showing quantification of the mitochondrial respiration data for basal respiration, maximal respiration, ATP production, and spare respiratory capacity obtained from three independent experiments. *, P≤0.03: MUL1(−/−) treated cells vs MUL1(−/−) con, #, P≤0.04: MUL1(−/−) con vs WT con. Data from three separate experiments are presented as means±SEM. FIG. 5E shows a Western blot analysis; the activity of HIF-1α and Akt2 proteins, in the presence or absence of the inhibitors, was verified by monitoring the expression of the respective target proteins, GLUT1 and P-GSK-3β S9. FIG. 5F show bar graphs illustrating the densitometrical analysis of the proteins shown in FIG. 5E, normalized against β-actin. Results shown as means±SD of three independent experiments. #, P≤0.03 vs WT con, *, P≤0.02: MUL1(−/−) treated cells vs MUL1(−/−) con.

FIGS. 6A-F show expanded ¹³C NMR spectra of HEK293 WT and HEK293 MUL1(−/−) cells. FIG. 6A shows the labeling pattern of lactate (C-2), glutamate (C-4), and glutamate (C-2). Lactate-C2 spectra indicating that pyruvate cycled through pyruvate kinase (PK) flux. The C2D12 and C2D23 represent the [1,2-¹³C] lactate and [2,3-¹³C] lactate isotopomers respectively, whereas 2Q signals represent [U-¹³C] lactate. S: singlet; D12, D23 and D45: doublet; Q: quartet. FIG. 6B is a bar graph showing the Glutamate ¹³C signal ratio obtained from ¹³C-NMR spectra of HEK293 WT, MUL1(−/−), MUL1(−/−)+Peri and MUL1(−/−)+CTM cells utilizing the [U-¹³C]glucose. P≤0.05: *, WT vs MUL1(−/−); #, WT vs MUL1(−/−)+Peri; ‡, WT vs MUL1(−/−)+CTM; ¶, MUL1(−/−) vs MUL1(−/−)+Peri; §, MUL1(−/−) vs MUL1(−/−)+CTM; £, MUL1(−/−)+Peri vs MUL1(−/−)+CTM. FIG. 6C shows a metabolic flux model demonstrating the ¹³C-labeling pattern of the metabolites derived from [U-¹³C]glucose. [U-¹³C]glucose derived [U-¹³C]pyruvate enters the TCA cycle through YPC (red dots) or PDH flux (green dots). Glucose oxidation through PDH flux labeled C4-C5 of glutamate (green dots) and C2-C3 of glutamate (red dots) through YPC flux. Key enzymatic steps involved in the metabolic flux model are as follows: (1) lactate dehydrogenase (LDH), (2) pyruvate kinase (PK), (3) pyruvate dehydrogenase (PDH), (4) pyruvate carboxylase (YPC), (5) glutamate dehydrogenase (GDH), (6) Phosphoenolpyruvate Carboxykinase (PEPCK), and (7) anaplerosis via succinyl-CoA (Ys). (Note: metabolic model demonstrates the ½ turn of the tricarboxylic acid (TCA) cycle). FIG. 6D is a bar graph showing the metabolic flux rates calculated from the ¹³C-isotopomers of glutamate observed in the ¹³C-NMR spectra. Signal areas obtained from the results of the peak fitting procedure were used as an input to a metabolic model and solved numerically using tcaCALC. All flux rates are referenced to a citrate synthase (CS) flux of 1 and is equivalent to Kreb's cycle flux. Statistical significance was P≤0.05: *, WT vs MUL1(−/−); #, WT vs MUL1(−/−)+Peri; ‡, WT vs MUL1(−/−)+CTM; ¶, MUL1(−/−) vs MUL1(−/−)+Peri; §, MUL1(−/−) vs MUL1(−/−)+CTM; £, MUL1(−/−)+Peri vs MUL1(−/−)+CTM.

FIGS. 6E-F show data illustrating that MUL1 inactivation in HeLa cells affects Akt2 and HIF-1α, protein levels, and metabolic flux. FIG. 6E is a bar graph showing glutamate ¹³C signal ratio obtained from ¹³C NMR spectra of HeLa WT and HeLa MUL1(−/−) cells utilizing the [U-¹³C] glucose. All signal ratios were calculated with respect to the total area of the corresponding glutamate resonance. FIG. 6F is a bar graph showing steady-state flux rates. The steady-state flux rates, relative to a Kreb's cycle flux, are calculated from the isotopomers of glutamate observed at carbon positions 2, 3, 4, and 5 in the ¹³C NMR spectra and modeled using tcaCALC. All flux rates are referenced to a citrate synthase (CS) flux of 1 and is equivalent to a Kreb's cycle flux. Pyruvate dehydrogenase (PDH), pyruvate carboxylase (YPC), pyruvate kinase (PK), and anaplerosis leading to succinyl-CoA (YS). P≤0.05 and indicated by *. HeLa WT vs MUL1 (−/−) cells. S, D, T, and Q are singlet, doublet, triplet, and quartet, respectively. Data is represented as mean±SEM.

FIGS. 7A-G show data illustrating that Akt2 and HIF-1α proteins contribute to support the metabolic phenotype of HEK293 MUL1(−/−) cells. FIG. 7A shows the glycolytic capacity in HEK293 WT and Akt2(−/−) cells before or after treatment with the HIF-1α activator DMOG (100 μM for 4 hours). Extra Cellular Acidification Rate (ECAR) was measured using the Seahorse analyzer. HEK293 MUL1(−/−) cells were used as a positive control. #, P≤0.035: vs WT con; *, P≤0.035: vs MUL1(−/−) con; ‡, P≤0.045: AKT2(−/−) con vs AKT2(−/−)+DMOG. FIG. 7B is a bar graph showing quantification of the glycolysis, glycolytic capacity and glycolytic reserve obtained from three independent experiments. Data from three separate experiments are presented as means±SEM. #, P≤0.03: vs WT con; *, P≤0.035: vs MUL1(−/−) con; ‡, P≤0.045: AKT2(−/−) con vs AKT2(−/−)+DMOG. FIG. 7C is a Western blot analysis showing the effect of HIF-1α activator, DMOG, on the expression levels of HIF-1α, GLUT1, Akt2, P-GSK-3β S9, in Akt2(−/−), or MUL1(−/−) cells. FIG. 7D is a bar graph showing the densitometrical analysis of the proteins shown in FIG. 7C, normalized against β-actin. Results shown as means±SD of three independent experiments. *, P≤0.03 vs WT con; #, P≤0.025 vs Akt2(−/−) con. FIG. 7E is a bar graph showing the glutamate ¹³C signal ratio obtained from ¹³C-NMR spectra of Akt2(−/−) and Akt2(−/−)+DMOG cells utilizing the [U-¹³C]glucose. (Note: S, D, T, and Q are singlet, doublet, triplet and quartet, respectively. All signal ratios were calculated with respect to the total area of the corresponding glutamate resonance. Data is represented as Mean±SEM). FIG. 7F shows metabolic flux rates calculated from the ¹³C-isotopomers of glutamate observed in the ¹³C-NMR spectra. Signal areas obtained from the results of the peak fitting procedure were used as an input to a metabolic model and solved numerically using tcaCALC. All flux rates are referenced to a citrate synthase (CS) flux of 1 and is equivalent to a Kreb's cycle flux. Statistical significance was P≤0.05: *, for Akt2(−/−) vs Akt2(−/−)+DMOG.

FIG. 7G shows data illustrating metabolic flux rates derived from the ¹³C-isotopomers of glutamate observed in the ¹³C-NMR spectra. Signal areas were used as an input to derive metabolic model and solved numerically using tcaCALC. All flux rates are referenced to a citrate synthase (CS) flux of 1 and is equivalent to a Kreb's cycle flux. Statistical significance was P≤0.05: *, WT vs MUL1(−/−); #, WT vs Akt2(−/−); ‡, WT vs Akt2(−/−)+DMOG; MUL1(−/−) vs Akt2(−/−); §, MUL1(−/−) vs Akt2(−/−)+DMOG, and £, Akt2(−/−) vs Akt2(−/−)+DMOG.

FIGS. 8A-L show data illustrating metabolomic comparison of HEK293 WT and HEK393 MUL1(−/−) cells. FIG. 8A is a 2D scores plot of the principal component analysis (PCA) from LC-MS positive ion mode data. The degree of variance is displayed in parentheses on each axis and the shaded areas indicate the 95% confidence regions based on the data points for each group. FIG. 8B is a 2D scores plot of partial least squares discriminant analysis (PLS-DA) from LC-MS positive ion mode data. The amount of variance explained is displayed in parentheses on each axis and the shaded areas indicate the 95% confidence regions based on the data points for each group in PLS-DA models. Supervised PLS-DA maximizes the separation between both groups using the group label and cross validation was also used to determine the optimal number of components required to build the PLS-DA model. The sum of squares captured by the model (R²) and the cross-validated R² (Q²) parameters were used to determine the robustness of the mathematical model. FIG. 8C is a graph showing corresponding variable importance in projection (VIP) scores plot of PLS-DA model B. Variable importance in projection (VIP) scores plots from PLS-DA model demonstrating the differences in the level of top 25 metabolites between HEK293 WT and MUL1(−/−) cells. FIG. 8D is a 2D scores plot of PCA from LC-MS negative ion mode data. FIG. 8E is a 2D scores plot of PLS-DA model from the LC-MS negative ion mode data. FIG. 8F is a graph showing PLS-DA variable importance in projection (VIP) scores plot of PLS-DA model E.

FIGS. 8G-H show data illustrating clustering of the top twenty-five metabolites between HEK293 WT and MUL1(−/−) cells. FIG. 8G illustrates clustering result shown as heatmap and demonstrating the differences in the level of the top 25 metabolites between HEK293 WT and MUL1(−/−) cells identified via PLS-DA and VIP scores, using LCMS data in the positive mode. FIG. 8H illustrates clustering result shown as heatmap that demonstrate the differences in the level of the top 25 metabolites between HEK293 WT and MUL1(−/−) cells identified via PLS-DA and VIP scores, using LC-MS data in the negative mode. (Note: The scale bar represents normalized intensities of the features. Heatmap visualizes a numerical table into a corresponding 2D color map to provide an overview of the data values, indicating level changes from low (cold) to high (hot) intensity.

FIG. 8I shows a semi-quantitative metabolomics panel. Box and Whisker plots displayed the differential level of significantly different metabolites between HEK293 WT and MUL1(−/−) cells.

FIGS. 8J-L show a metabolite set enrichment analysis. FIG. 8J illustrates an interactive network of pathways showing the connections between individual pathways. Significantly different metabolites between HEK293 WT and MUL1(−/−) cells were used for metabolite set enrichment analysis. It is used to investigate if a group of functionally related metabolites are significantly enriched, eliminating the need to preselect compounds based on some arbitrary cut-off threshold. FIG. 8K illustrates the top 25 enrichment overview of MSEA. FIG. 8L is a dot plot of the enrichment analysis results. The size of the circles per metabolite set represents the Enrichment Ratio and the color represents the P-value.

FIGS. 9A-D show data illustrating RNA-sequencing and joint pathway analysis in HEK293 MUL1(−/−). FIG. 9A is a volcano plot showing global transcriptional change across the groups compared. Each data point in the volcano plot represents a gene. The log 2 fold change of each gene is represented on the x-axis and the log 10 of its adjusted p-value is on the y-axis. Genes with an adjusted p-value less than 0.05 (P<0.05) and a log 2 fold change greater than 1 are indicated by red dots. These represent up-regulated genes. Genes with an adjusted P<0.05 and a log 2 fold change less than −1 are indicated by green dots. These represent down-regulated genes. FIG. 9B is a heatmap of the top 50 differentially expressed genes shown for HE293 WT and MUL1(−/−) cells. FIG. 9C is a heatmap of the differentially expressed genes involved in cellular metabolism. Most of the genes related to the glucose and glutathione metabolism are downregulated in HEK293 MUL1(−/−) cells. The genes related to fatty acid and retinoic acid metabolism are upregulated as well as downregulated in HEK293 MUL1(−/−) cells. FIG. 9D is a graph showing the fold change in HEK293 MUL1(−/−) cells versus the WT control was used as an input to preform joint metabolic pathway analysis employing gene and metabolite from uniport protein ID and HMDB ID, respectively. Metabolic pathways containing both metabolites and metabolic genes were used for the integrative pathway analysis along with Fisher's exact test for the enrichment analysis. The metabolic pathways with higher P-values and pathway impacts were significantly altered in HEK293 MUL1(−/−) cells.

FIGS. 10A-G show data illustrating a lipidomic comparison of HEK293 WT and HEK293 MUL1(−/−) cells. FIG. 10A is a graph showing high-resolution LC-MS/MS-based lipidomic profiling of lipid classes i.e. triglycerides (TGs), diacylglycerols (DGs), phosphatidylcholines (PCs), phosphatidylethanolamines (PEs) and ceramides in the HEK293 WT and MUL1(−/−) cells demonstrating significant changes in TGs and DGs lipid classes. Lipidomic profiling of ten highest abundant (FIG. 10B) TGs, (FIG. 10C) DGs, (FIG. 10D) PCs, (FIG. 10E) PEs and (FIG. 10F) ceramides in WT and MUL1(−/−) cells, providing more insights into the changes in the level of individual lipids. HEK293 MUL1(−/−) cells demonstrate significant changes in the level of several lipids with respect to the WT control cells. (Note: Values for each bar are expressed as mean±SEM (n=4) and individual lipid intensity was normalized to the total lipid intensity of each sample. P≤0.05: a: WT vs MUL1(−/−). FIG. 10G shows a lipid ontology enrichment analysis of the MUL1(−/−) cells. Enrichment analysis of MUL1(−/−) vs WT cells in the “ranking mode”. The gray vertical lines indicate the cut-off value of significantly up (red bars) and down (blue bars) lipids in MUL1(−/−) cells with respect to WT (q<0.05). The bar colors are scaled with the enrichment (−log FDR q-values). Lipid Ontology (LION): a web-based interface was used for the identification of lipid-associated terms in lipidomes.

FIG. 11 is a schematic diagram illustrating the potential MUL1-dependent pathway in the regulation of metabolism. The metabolism in MUL1(−/−) cells can be summarized as increased glycolytic rate, increased anaplerotic (Y_{PC}) and PK fluxes, and increase in the lipid storage. This regulation in metabolism is mediated through Akt2 and HIF-1α proteins as suggested by the inhibition of these proteins, using perifosine and chetomin, respectively. The individual roles of Akt2 and HIF-1α are established in Akt2(−/−) cells resulting in reduced PK flux, whereas activation of HIF-1α by DMOG increases glycolysis, as well as PK and Y_{PC} fluxes compared to Akt2(−/−) cells.

FIG. 12 is a photo comparing size of a wild type (WT) mouse to size of a MUL1 knockout (KO) mouse. MUL1 KO mice are slightly smaller but otherwise healthy.

FIGS. 13A-D are photos comparing size of WT mice to size of MUL1 KO mice. FIGS. 13A-B show MUL1(−/−) male mice compared to MUL1(+/+) male mice and FIGS. 13C-D show MUL1(−/−) female mice compared to MUL1(+/+) female mice.

FIGS. 14A-B are micrographs comparing a muscle section of a MUL1(+/+) mouse (FIG. 14A) to a muscle section of a MUL1(−/−) mouse (FIG. 14B).

FIGS. 15A-C are Western blot analysis comparing protein expression in tissues of MUL1(+/+) mice to protein expression in tissues of MUL1(−/−) mice. Proteins are HIF-1α, UBXN7, GLUT1, MUL1, and GAPDH (control). Brain tissue is compared in FIG. 15A; heart tissue is compared in FIG. 15B; and liver tissue is compared in FIG. 15C.

FIG. 16 is an illustration showing and explaining the Promethion Cage.

FIGS. 17A-F show data illustrating whole body metabolic studies of MUL1(−/−) mice and WT mice on a high-fat diet (HFD) using 16-channel Promethion system gas-tight metabolic cage of FIG. 16. FIGS. 17A-F show a comparison of characteristics of MUL1(−/−) mice fed a high fat diet (HFD) to characteristics of MUL1(+/+) mice fed a high fat diet. N=4 KO; N=4 WT. Food consumption is compared in FIG. 17A; wheel running is compared in FIG. 17B; activity/locomotion is compared in FIG. 17C; oxygen (O₂) consumption is compared in FIG. 17D; carbon dioxide (CO₂) production is compared in FIG. 17E; body weight is compared in FIG. 17F. Representative data were collected over a period of 24 hours; night (dark) and day (light). *p<0.05 vs WT group n=3

FIGS. 18A-H show data illustrating phenotype of MUL1 KO mice on high-fat diet (HFD) and provides evidence that MUL1 inactivation protects against HFD-induced obesity. FIG. 18A—Mul1(+/+) and Mul1(−/−) mice after 16 weeks on HFD. FIG. 18B—Body weight of male and female animals was monitored every 2 weeks during HFD period. FIG. 18C-Glucose tolerance test (GTT). FIG. 18D—insulin tolerance test (ITT) was performed on male mice after 16 weeks of HFD (n=4 per group). FIG. 18 E—tissue samples: White adipose inguinal (iWAT), subcutaneous (sWAT), and mesenteric (mWAT). FIG. 18F—H&E and Oil red (ORO) staining on liver tissue sections. FIG. 18G—Western blot analysis of liver extracts to monitor the expression of proteins involved in MUL1 pathway. FIG. 18H—Densitometric analysis of protein expression from FIG. 18G. *p<0.04 vs Mul1(+/+).

FIGS. 19A-F show data illustrating that Mul1(−/−) mice are protected against HFD (high fat diet)-induced obesity and other metabolic consequences. FIG. 19A—Representative images of Mul1(+/+) and Mul1(−/−) mice (male and female) maintained on HFD to highlight the size difference between these animals. All mice were fed a regular chow until they were 8 weeks old. They were then placed on a HFD, in which at least 60% of the calories are obtained from fat, for 16 weeks. FIG. 19B—is a graph showing body weight of Mul1(+/+) and Mul1(−/−) male and female animal groups that were monitored every 2 weeks during the HFD period. FIG. 19C—is a graph showing results of a glucose tolerance test. FIG. 19D—is a graph showing result of an insulin tolerance test that was performed on male mice after 16 weeks of HFD (n=5 per each group). FIG. 19E-Representative images to highlight the difference in white adipose tissue volume between the Mul1(+/+) and Mul1(−/−) mice: inguinal (iWAT), subcutaneous (sWAT), and mesenteric (mWAT). FIG. 19F—Histology was performed on liver tissues of Mul1(+/+) and Mul1(−/−) animals on HFD; Representative image of Hematoxylin and eosin (H&E) and Oil red O (ORO) staining of liver tissues. All data are presented as the mean of individuals in each group±S. D. of three independent experiments, * p<0.05 and ** p<0.01.

FIGS. 20A-G Mul1(−/−) mice on HFD have increased respiration and energy expenditure. Mice were individually housed in Promethion system metabolic cages at 22° C. for 6 days of data collection. Animal groups were maintained on ND or HFD as indicated, throughout the assay. Representative data were selected over a period of 48 hours (dark/light cycle). FIG. 20A-Oxygen consumption graph, FIG. 20B—Oxygen consumption plot bar, FIG. 20C—Energy expenditure line graph, and FIG. 20D-Energy expenditure plot bar. FIG. 20E-Average food intake FIG. 20E—activity measured by pedestrian locomotion, and FIG. 20G-Body weight. Data are presented as means±S.E.M, p value is indicated for each measurement (n=5-7). Data analysis was performed using CalR software: grey bars indicate period of darkness, white bars day light. The significant code is indicated as *p<0.05, **<0.01 and ***<0.001 Mul1(−/−) vs Mul1(+/+).

FIGS. 21A-I Thermoregulation and cold tolerance are intact in Mul1(−/−) mice. Representative data were collected from Mul1(+/+) and Mul1(−/−) mice on ND over a period of 48 hours at 22° C. followed by a cold challenge at 4° C. for another 48 hours. FIG. 21A-Oxygen consumption line graph and FIG. 21B—Oxygen consumption plot bar. FIG. 21C-Respiratory exchange line graph and FIG. 21D—Respiratory exchange plot bar. FIG. 21E-Energy expenditure line graph and FIG. 21F—Energy expenditure plot bar. FIG. 21G—Body temperature of animals recorded during the cold exposure over 10 hours at 4° C. FIG. 21H—Western blot analysis of BAT extracts isolated from animals following cold exposure to monitor the expression of UCP1, and MUL1 proteins using specific antibodies. GAPDH protein expression was used to verify equal loading on each lane. FIG. 21I—Body weight before and after cold exposure. Data plotted as the mean±S.E.M, n=4. Data analysis was performed using CalR software: grey bars indicate the period of darkness, white bars the daylight.

FIGS. 22A-H Metabolomic analysis of Mul1(−/−) mouse liver on HFD. FIG. 22A and FIG. 22D score plots of principal component analysis (PCA) from LC-MS data of positive and negative ion modes, respectively. The amount of variance explained is shown in parentheses on each axis and the shaded area indicates the 95% confidence region. FIG. 22B and FIG. 22E score plots of partial least squares discriminant analysis (PLS-DA) from similar datasets. The amount of variance is displayed in parentheses on each axis and the shaded areas indicate the 95% confidence regions based on the data points for each group in PLS-DA models. FIG. 22C and FIG. 22F—variable importance in projection (VIP) scores plot derived from PLS-DA models, FIG. 22B and FIG. 22E, respectively. VIP scores plots from PLS-DA models demonstrated the differences in the level of top 25 metabolites between Mul1(+/+) and MUL1(−/−) liver. FIG. 22G—Bar diagram demonstrating the levels of significantly different metabolites between the liver of Mul1(+/+) and Mul1(−/−) animals on HFD. The metabolites shown in the plot were identified by LC-MS positive ion mode and FIG. 22H-metabolite signal intensity was normalized to per mg of the liver tissue weight. The metabolites shown in the plot were identified by LC-MS negative ion mode and metabolite signal intensity was normalized to per mg of the liver tissue weight. The p value of the all significantly different metabolites is indicated *≤0.05 Mul1(+/+) vs MUL1(−/−) liver.

FIGS. 23A-G Lipidomic profiling of Mul1(−/−) mouse liver on HFD. FIG. 23A—High-resolution LC-MS/MS based profiling of total lipids and lipid classes (triglycerides (TGs), diacylglycerols (DGs), phosphatidylcholines (PCs), phosphatidylethanolamines (PEs) and ceramides) in Mul1(+/+) control and Mul1(−/−) mice liver. Profiling of ten highest abundant triglycerides (TGs) FIG. 23 B, diacylglycerols (DGs) FIG. 23C, phosphatidylcholines (PCs) FIG. 23D, phosphatidylethanolamines (PEs) FIG. 23E, and ceramides FIG. 23F in Mul1(+/+) and Mul1(−/−) mice liver, providing more insights into the profiles of individual lipids. (Note: Values for each bar are expressed as mean±SEM (n=4) and individual lipid intensity was normalized to per mg of liver tissue weight. *≤0.05 Mul1(+/+) vs Mul1(−/−) mice liver. FIG. 23G—Lipid Ontology (LION) enrichment analysis of Mul1(−/−) mice liver. Enrichment analysis of Mul1(−/−) and Mul1(+/+) liver in the “ranking mode”. The gray vertical lines indicate the cut-off value of significantly up (red bars) and down (blue bars) lipids in Mul1(−/−) mice liver with respect to Mul1(+/+) (q<0.05).

FIGS. 24A-C Differential gene expression identifies genes involved in fatty acid synthesis that are downregulated in Mul1(−/−) mice HFD-liver. FIG. 24A—Heatmap of the differentially mRNA expressed genes involved in fatty acid biosynthesis and metabolism; most of the genes in this group are downregulated and only few are upregulated, FIG. 24B, top four ranked differentially expressed genes of which three are upregulated: TFF3, ASNS and CYP2A4, and one, the SCD1, is downregulated. FIG. 24C-Joint metabolic pathway analysis, employing gene and metabolite with uniport protein ID and HMDB ID, respectively with the fold change in Mul1(+/+) and Mul1(−/−) liver. Metabolic pathways including metabolites and metabolic genes were used for the integrative pathway analysis with Fisher's exact test for the enrichment analysis. The metabolic pathways with higher p value as well as pathway impact were significantly perturbed in Mul1(−/−) liver (*p value <0.03).

FIGS. 25A-G The SCD1 pathway of lipogenesis is dysregulated in the absence of MUL1. Liver and sWAT tissue extracts were prepared from mice on ND or HFD as described in Methods. Equal amounts of extract were analyzed by SDS-PAGE and Western blot analysis using specific antibodies. FIG. 25A—expression of SCD1 protein in sWAT and FIG. 25B-liver tissues from Mul1(+/+) and Mul1(−/−) mice. β-actin was used to verify equal loading in each lane. FIG. 25C—Graph represents the densitometrical analysis of the SCD1 expression (left) and MUL1 expression (right) normalized against β-actin. *p≤0.036 vs ND(+/+); #p≤0.011 vs HFD(+/+). FIG. 25D—The expression of CPT1, AMPK, pAMPK, was monitored in ND and HFD-liver by Western blot analysis. FIG. 25E—Graph represents the densitometrical analysis of proteins shown in D normalized against β-actin. *p≤0.02 vs ND (+/+); #p≤0.02 vs HFD (+/+). FIG. 25F—The regulation of FASN, ACC1 and pACC1 proteins was monitored in ND and HFD-liver; HSP90 expression was used to verify equal loading on each lane. FIG. 25G—Graph shows the protein/HSP90 ratio after densitometry analysis. *p≤0.01 vs ND (+/+); #p≤0.014 vs HFD (+/+). Results are shown as means±S. D. of at least three independent experiments.

FIG. 26 Schematic diagram of the proposed lipogenic pathway, dysregulated in the absence of MUL1. The absence of MUL1 leads to a decrease in the expression of SCD1 and furthermore, inhibits the induction of this protein that occurs with HFD. This results in the activation and accumulation of phosphorylated-AMPK (pAMPK) which downregulates the protein level of ACC1 and FASN enzymes. In addition, pAMPK1 increases expression of CPT1 on the OMM that is involved in the transport of fatty acids into the mitochondria for (3-oxidation. CPT1 is also regulated by the level of Malonyl-CoA, and decreased levels of this metabolite, due to downregulation of ACC1, leads to further activation of CPT1. The low level of malonyl-CoA and the reduction in FASN protein level invariably inhibit the synthesis of fatty acids. In addition, downregulation of SCD1 decreases the production of monounsaturated fatty acids (MUFA) leading to limited lipogenesis.

FIGS. 27A-C Mul1(−/−) mice on ND present a metabolic phenotype. FIG. 27A-representative image of male Mul1(+/+) and Mul1(−/−) animals (24 weeks old) on a ND. FIG. 27B—glucose tolerance test and FIG. 27C—insulin tolerance test was performed on male mice after 16 weeks on ND (n=4 male and n=4 male per group). Data presented as the mean of individuals in each group±S. D. of three independent experiments.

FIGS. 28A-G Whole body metabolic studies using Mul1(−/−) mice on ND. Representative data were selected over a period of 48 hours (dark/light cycle). FIG. 28A-Oxygen consumption graph, FIG. 28B-Oxygen consumption plot bar, FIG. 28C—Energy expenditure line graph as well as FIG. 28D—Energy expenditure plot bar. FIG. 28E—Average food intake FIG. 28F—activity measured by pedestrian locomotion, and FIG. 28G—Body weight. Data analysis was performed using CalR software: grey bars indicate period of darkness, white bars day light. The significant code is indicated as *p<0.05 Mul1(−/−) vs Mul1(+/+) group n=4.

FIGS. 29A-C Lipidomic study of Mul1(+/+) and Mul1(−/−) mouse liver on HFD. FIG. 29A—and FIG. 29B—principal component analysis (PCA) and partial least squares discriminant analysis (PLS-DA) scores plots from LC-MS lipidomic data of Mul1(+/+) and MUL1(−/−) mice liver. The amount of variance is shown in parentheses on each axis of PCA and PLS-DA. The shaded area indicated the 95% confidence regions based on the data points for each group in PCA and PLSDA models. FIG. 29C—heatmap showing the clustering results as well as the differences in the level of top 50 lipids between Mul1(+/+) and Mul1(−/−) mouse liver.

FIG. 30A-B Human liver cells (HepG2) FIG. 30A—wild type (WT) and FIG. 30B—HepG2 cells were MUL1 was inactivated using CRISPR-Cas9 [HepG2-MUL1(−/−)]. Cells were treated with 200 μM of oleic acid (OA) for 24 hours to simulate high fat diet (HFD) and stained with Mitotracker-Red (to stain mitochondria) and Bodipy-green for lipid droplet visualization. Images were obtained using a confocal microscopy (Leica SP5). Inactivation of MUL1 in HepG2 cells significantly decreases the amount of lipid accumulation.

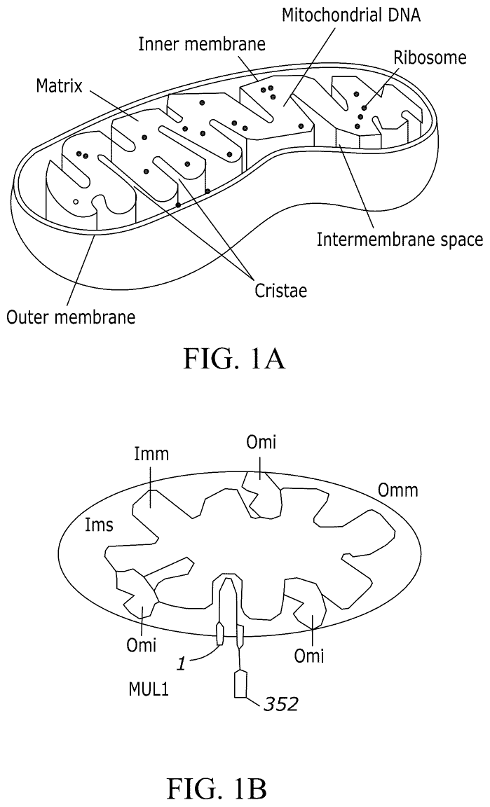

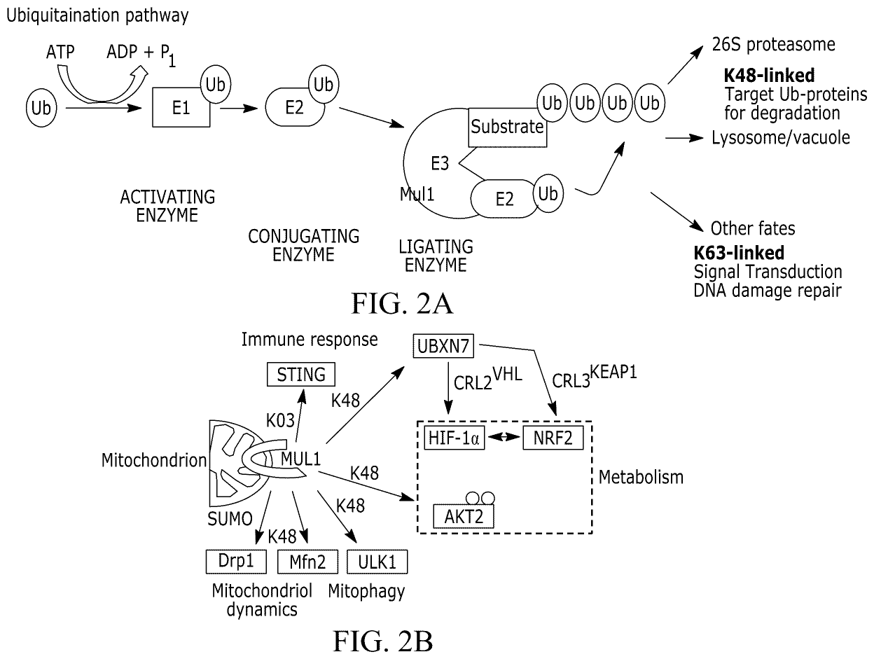

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described regulators, modulators, inhibitors, genetic profiles, compositions, kits, pharmaceutical compositions and/or methods along with any further application of the principles of the invention as described herein, are contemplated as would normally occur to one skilled in the art to which the invention relates.

### Introduction to Experimentation—Study One—In Vitro Aspects

The primary function of mitochondria is to meet the energy demand by providing ATP through oxidative phosphorylation (OXPHOS); in addition, they have a central role in cellular homeostasis, cell death, and the regulation of metabolism (138; 129; 156). Mitochondria can modify their bioenergetic and biosynthetic function to meet the metabolic demands of the cell, in response to changes in the physiological environment (nutrient stress) or low O₂ levels (hypoxia) (134; 146; 135). The regulation of metabolism requires continuous and effective communication between the mitochondria with the rest of the cell which involves numerous proteins with diverse function (160). MUL1 (also known as Mulan, MAPL, GIDE, and HADES), is a mitochondrial E3 ubiquitin ligase, and one of only three E3 ligases found in mitochondria (the other two are MARCH5 and RNF185) (6; 167; 23; 166; 30; 60; 59; 33). MUL1 is anchored in the outer mitochondrial membrane (OMM) with two transmembrane domains, a large intermembrane domain (IMD), and a RING finger domain facing the cytoplasm, which is responsible for its ligase function. Based on its topography, MUL1 can convey changing conditions within the mitochondria to ubiquitinate specific substrates in the cytoplasm. MUL1 is able to K48- or K63-ubiquitinate, as well as SUMOylate a number of specific substrates and its function has been implicated in the regulation of mitophagy, cell death, mitochondrial dynamics, and innate immune response (167; 23; 168; 59; 37; 39; 25; 26; 22; 169). The studies described herein are focused on the MUL1-mediated K48-polyubiquitination that invariably targets its substrates for proteasomal degradation and therefore regulates their protein level (136). A new function of MUL1 in the regulation of metabolism is described herein. Inactivation of MUL1 ligase has a profound effect on glycolysis, lipid metabolism, mitochondrial anaplerotic fluxes, and pyruvate cycling. The mediators of MUL1 function in metabolism, the transcription factor HIF-1α, and the serine/threonine kinase Akt2 were characterized. The previous studies have shown that MUL1 can regulate the HIF-1α protein to support a glycolytic phenotype in cells (22; 34). In addition, the Akt2 protein, that is implicated in the regulation of metabolism, is also a known substrate of MUL1 (28; 27; 40). Results presented herein show MUL1, through K48 polyubiquitination, co-regulates both Akt2 and HIF-1α protein level to maintain a normal metabolic state. Inactivation of MUL1 leads to accumulation and activation of both Akt2 and HIF-1α proteins and drives a unique metabolic state that has not been previously characterized. The new function of mitochondrial MUL1 E3 ubiquitin ligase in mitochondrial respiration and the regulation of metabolism has been identified and characterized as described herein. Furthermore, the MUL1 involvement in metabolism mediated by the concurrent regulation of Akt2 and HIF-1α proteins has been delineated.

### Materials and Methods—Experiments Using Cells

### Cell Culture and Chemicals

HEK293 wild type (WT), HEK293 MUL1(−/−) and HEK293 Akt2(−/−) cells were grown using Dulbecco's modified Eagle's medium (DMEM high glucose, sodium pyruvate) supplemented with 10% fetal calf serum (Atlanta Biologicals), 2 mM L-glutamine, 50 units/ml penicillin, and 50 μg/ml streptomycin (Thermo Fisher Scientific, USA). HeLa cells were maintained in Dulbecco's modified Eagle's medium supplemented with 10% fetal bovine serum (Atlanta Biological), 50 units/ml penicillin, and 50 μg/ml streptomycin. Chemicals: Perifosine, chetomin, DMOG, oligomycin, FCCP, antimycin A, rotenone, (SIGMA) were dissolved in DMSO, stored at −80° C., and used at the indicated concentrations. XF DMEM media pH 7.4, glucose, pyruvate, and glutamine solutions (Agilent Technologies). DMSO (0.1%) was used as vehicle control.

### SDS-PAGE and Western Blot Analysis

Control (untreated) as well as cells treated with perifosine, chetomin or DMOG were lysed using a Triton X-100 based lysis buffer (1% Triton X-100, 10% glycerol, 150 mM NaCl, 20 mM Tris (pH 7.5), 2 mM EDTA) in the presence of protease inhibitors (Thermo Fisher Scientific). Approximately 40 μg of whole cell extract was resuspended in SDS sample buffer, boiled for 4 minutes, and analyzed by SDS-PAGE (12% or 15% gels), then transferred onto PVDF membranes (Genesee) using a semi-dry cell transfer blot (Bio-Rad). Nonfat dry milk (4%) or 5% BSA (for Akt2 and phospho-specific antibodies) in TBST buffer (25 mM Tris-HCl, pH 8.0, 125 mM NaCl, 0.1% Tween 20) were used to block nonspecific binding of the membrane. The membranes were incubated with the indicated primary antibodies: HIF-1α (Bioss Antibodies, 1:2,000), AKT (pan), Akt1, Akt2, Akt3, phospho-Akt-T308, phospho-GSK-3β-S9, ULK1 (Cell Signaling Technology 1:2,000), GLUT1, (ABclonal 1:2,000), MFN2, and β-actin (Santa Cruz Biotechnology, 1:3,000). MUL1 and UBXN7 rabbit polyclonal antibodies were homegrown and used at 1:5,000 dilution. Secondary peroxidase-conjugated goat anti-rabbit or goat anti-mouse antibodies (Jackson ImmunoResearch) were used at 1:10,000 dilution. The membrane was visualized by enhanced chemiluminescence (ECL) (Thermo Fisher Scientific).

### Generation of HEK293 Akt2(−/−) and HeLa Mul1(−/−) Using CRISPR/Cas9 Gene Editing

To ablate Akt2 expression, the target sequence 5′-GACCCCATGGACTACAAGTG-3′ (SEQ ID NO:1) located in exon 4 was selected using the CRISPOR program (doi: 10.1186/s13059-016-1012-2; http://crispor.tefor.net) and cloned into pSpCas9(BB)-2A-GFP (PX458) vector (Addgene) as previously described (155). To knockout Mul1 in HeLa cells similar method was used with the specific target sequence 5′-GCCGCCGTCATGGAGAGCGG-3′ (SEQ ID NO:2) in exon1. The resulting vectors (PX458-Akt2-target and PX458-Mul1-target) or the empty PX458 control vector were transfected into HEK293 or HeLa cells respectively. Forty-eight hours later, single GFP-positive cells were sorted into 96 well plates using a FACS ARIA II sorter (BD Biosciences). The clones were expanded and Akt2 or MUL1 protein expression was monitored by Western blot analysis. In addition, genomic DNA was isolated and used for PCR amplification and DNA sequencing to verify deletion of the target sequence surrounding exon 4 of the Akt2 gene or exon 1 of the Mul1 gene. Three independent HEK293 Akt2(−/−) and three HeLa MUL1(−/−) clones as well as two HEK293 or HeLa WT control clones (transfected with PX458 empty vector) were chosen for further experiments. The HEK293 MUL1(−/−) cells have been previously described (159; 22; 34).

### Glycolytic Stress Assay

To monitor glycolysis in the cell lines, the Glycolysis Stress Test was performed using a XFe24 Extracellular Flux Analyzer (Agilent Technologies). HEK293 WT, MUL1(−/−) and Akt2(−/−) cells were seeded in triplicates on poly-lysine D-coated XFe24 microplates at a density of 60,000 cells per well in assay medium (XF DMEM pH 7.4 containing 2 mM glutamine and 1 mM sodium pyruvate without glucose) for one hour in a CO₂-free incubator. Extracellular acidification rate (ECAR) was measured under basal conditions and again after sequential injections of glucose (10 mM) in port A, ATP synthase inhibitor oligomycin (1.5 μM) in port B, and the glycolysis inhibitor 2-deoxyglucose (2-DG) (300 mM) in port C. Basal glycolysis was assessed by recording three measurements, following the addition of each compound by a 3-2-3 mix/measurement cycle. Glycolysis is the ECAR after the addition of glucose. Glycolytic capacity is the increase in ECAR after the injection of oligomycin following glucose. Glycolytic reserve is the difference between glycolytic capacity and glycolysis. Data analysis was performed using Report Generators software for Glycolysis Stress Test (Agilent Technologies).

### Mitochondrial Stress Assay

Mitochondrial stress assay was performed using an XFe24 Extracellular Flux Analyzer (Agilent Technologies) following the workflow provided by the manufacturer's instructions. Briefly, for Oxygen Consumption Rate (OCR) measurements, HEK293 WT and MUL1(−/−) cells were seeded in triplicates on poly-lysine D-coated XF24 microplates at a density of approximately 60,000 cells per well in assay medium (XF DMEM medium pH 7.4 supplemented with 10 mM glucose, 2 mM glutamine, and 1 mM pyruvate), followed by incubation at 37° C. in CO₂-free incubator for 60 minutes. Three baseline measurements were recorded before the injection of the following compounds: 1.5 μM of oligomycin in port A, 1.0 μM of FCCP in port B and 0.5 μM rotenone/antimycin in port C. Data analysis was performed using Cell Mito Stress Test Report Generators software (Agilent Technologies).

Stable Isotope Tracer Experiment with Cells for NMR Spectroscopy

For NMR analysis, approximately 20×10⁶ HEK293 WT, MUL1(−/−), or Akt2(−/−) cells were grown to confluency following treatment with perifosine, or chetomin inhibitors as well as with the HIF-1α activator DMOG and DMSO was used as vehicle control. Freshly prepared [U-¹³C]glucose tracer containing medium (glucose free DMEM with 10% dialyzed FBS and 15 mM [U-¹³C]glucose) was added to the cells for 6 hours at 37° C. Cells were scraped from the plates, washed twice with cold PBS, and immediately snap frozen in liquid nitrogen (161).

### Extraction of the Metabolites and NMR Analysis

Cell pellets were bead-homogenized in 1 ml acetonitrile:isopropanol:water (3:3:2, v:v:v), centrifuged at 10,000×g for 15 min at 4° C. and the supernatant removed and dried in a speedvac. Dried cell extracts of each sample were re-suspended in 0.5 ml of acetonitrile: water (1:1, v:v) solution, vortexed, and centrifuged for 15 min at 10,000×g at 4° C. The supernatant was transferred to a new tube and dried in a speedvac The dried extract of each cell pellet was re-suspended in phosphate buffer (prepared in Deuterium oxide (D₂O)) for NMR analysis. The final volume of the sample (50 μl) consisted of 90% (v/v) deuterated 50 mM sodium phosphate buffer (pH 7), with 2 mM ethylene diamine tetra-acetic acid (EDTA), whereas 10% (v/v) was occupied by 5 mM D₆-4,4-dimethyl-4-silapentane-1-sulfonic acid (DSS) and 0.2% sodium azide (NaN₃) in D₂O (147; 130).

### NMR Spectroscopy and Data Processing

¹H-NMR spectra were collected on an 800 MHz NMR, equipped with a 5 mm TXI CryoProbe and Avance III console (Bruker Biospin), using TopSpin software (version 3.6.3). The ¹H-NMR spectra were acquired using a noesypr1d pulse sequence consisting of a 1 sec relaxation delay (dl) and a mixing time of 100 ms. A 4 sec acquisition time (AQ) over a spectral width (sw) of 12 ppm gave a final time-to-repeat of 5.1 sec. A total of 64 scans were acquired for each spectrum. The conventional ¹H-decoupled ¹³C-NMR spectra were acquired for each sample at 150.13 MHz, using a ¹³C-optimized 1.5 mm high temperature superconducting (HTS) probe on an Agilent NMR system with the magnetic field strength of 14 tesla. ¹³C-NMR spectra were recorded using an acquisition time (AQ) of 1.5 sec, relaxation delay (dl) of 1.5 sec, flip angle of 45°, acquired size (TD) of 54 k with the spectral width of 250 ppm (Ramaswamy et al., 2013). All NMR spectra were acquired at room temperature (25° C.). NMR data processing was performed in MestReNova software (v14.0.1-23284, Mestrelab Research S.L.). ¹H-NMR spectra were Fourier Transformed (FT) with a line-broadening factor of 0.5 Hz, zero filling to 65,536 data points, and baseline correction with the spline method. ¹³C-NMR spectra were processed with the following processing parameters: zero-filling to 128 k data points, exponential line broadening of 0.5 Hz, manual phase correction, and Whittaker smoother method for baseline correction.

### ¹³C-NMR Analysis of the Glutamate Isotopomers

The positional ¹³C-isotopomer distribution pattern of glutamate was determined by ¹³C-NMR. ¹³C-NMR resonances of various isotopomers of glutamate are identified from the ¹³C-¹³C J-coupling constants and quantified by line fitting to the glutamate resonances at C2, C3, C4, and C5 positions. The ¹³C-labeling was described first by the position of the ¹³C label, then by the multiplicity of the resonance and its origin. For example, C2S would denote the isotopomer of glutamate labeled only at the C2 position. The descriptor C2D12 describes the resonance of the C2 position that is split into a doublet (D), from coupling to an adjacent ¹³C label at the C1 position. The descriptor C2D23 denotes the resonance of the C2 position that is split into a doublet (D), from coupling to an adjacent ¹³C label at the C3 position. When a ¹³C-labeled C2 is flanked by two other neighboring ¹³C-labeled positions (C1 and C3), the resonance is split twice by the J-coupling into a doublet of doublets, or a quartet (Q), denoted C2Q. Similarly, other carbon resonances of glutamate were assigned, and peak areas were extracted for each of the isotopomers.

Fluxomic Analysis Using ¹³C-Isotopomer Data in tcaCALC

Metabolic flux relative to Krebs cycle turnover was calculated from the ¹³C-isotopomers of glutamate observed at carbon positions 2, 3, 4, and 5 in the ¹³C-NMR spectra of each cell sample. The tcaCALC program in MATLAB was utilized to perform an isotopomer analysis to estimate relative pathway fluxes (128). The metabolic model provides the best fit to our ¹³C-NMR data by calculating the relative fluxes of pyruvate dehydrogenase (PDH), pyruvate carboxylase (Y_{PC}), pyruvate kinase (PK), and anaplerosis leading to succinyl-CoA (Ys). All flux rates are referenced to a citrate synthase (CS) flux of 1 and is equivalent to Kreb's cycle flux. The input file contains the peak area ratios with initial parameters for PDH, PK, Y_{PC}, and Ys fluxes of 0.2, 03, 0.1, and 0.2, respectively.

### LC-MS Analysis for Metabolomics and Lipidomic

Cell pellets of HEK293 WT or MUL1(−/−) cells were subjected to the Folch extraction procedure, which resulted in two phases i.e., aqueous phase and chloroform phase, containing polar metabolites and non-polar lipids, respectively. The aqueous phase was analyzed using the Thermo Q-Exactive Orbitrap mass spectrometer with Dionex UHPLC and autosampler. All samples were analyzed in positive and negative heated electrospray ionization (HESI) with a mass resolution of 35,000 at m/z 200 as separate injections. Separation was achieved on an ACE 18-pfp 100×2.1 mm, 2 μm column with mobile phase A as 0.1% formic acid in water and mobile phase B was acetonitrile. The flow rate was 350 μl/min with a column temperature of 25° C. A 4 μl sample solution was injected for negative and 2 μl for positive ion mode LC-MS analysis. MZmine software was used to identify features, deisotopes, align features and perform gap filling. The metabolomics data were searched against SECIM's internal retention time metabolite library. LC-MS analysis for lipidomics was performed on the chloroform phase of the Folch extracted cell samples, utilizing a Thermo Q-Exactive Orbitrap mass spectrometer with Dionex UHPLC and autosampler. All samples were analyzed in HESI source with a mass resolution of 35,000 at m/z 200. LC separation was achieved on an Acquity BEH C18 1.7 μm, 100×2.1 mm column. The mobile phase A (60:40 Acetonitrile:10 mM Ammonium formate with 0.1% formic acid water) and mobile phase B (90:8:2 2-propanol:acetonitrile:10 mM ammonium formate with 0.1% formic acid in water) was used for elution of the lipids. The flow rate was 500 μl/min, and the column temperature was maintained at 50° C. Lipidomics data were analyzed using LipidMatch software (187).

### Metabolomics Data Analysis

The web-based tool: MetaboAnalyst (https://www.metaboanalyst.ca) was used for metabolomic data analysis, interpretation, and integration of the joint metabolic pathway analysis of genomics and metabolomics data. For multivariate statistical analysis, LC-MS data from positive and negative ion modes were imported into MetaboAnalyst software and subjected to normalization by the sum of the intensities, data transformation via log transformation, and pareto scaling. Joint metabolic pathway analysis was performed with gene and metabolite using uniport protein ID and HMDB ID, respectively. The fold change for MUL1(−/−) compared to WT genes and metabolites was used as an input to perform joint pathway analysis. Metabolic pathways containing both metabolites and genes were used for the integrative pathway analysis along with Fisher's exact test for the enrichment analysis. Simultaneously, an interactive network of pathway analysis, showing the connections between pathways and an individual pathway was carried out, using significantly different metabolites between HEK293 WT and MUL1(−/−) cells using results from both positive and negative mode ionization. A quantitative metabolomics panel of significantly different metabolites between HEK293 WT and MUL1(−/−) cells were created, employing statistical analysis via t-test in MetaboAnalyst (188). Box and Whisker plots were prepared in GraphPad prism, which displayed the differential level of significantly different metabolites between HEK293 WT and MUL1(−/−) cells

### Lipidomic Data Analysis

High-resolution LC-MS/MS-based lipidomic profiling of lipid classes was carried out using the normalized individual lipid intensity to the total lipid intensity of each sample. The values for each lipid class and the top 10 most abundant lipids in each class are expressed as mean±SEM (n=4). Lion term enrichment analysis of HEK293 WT versus MUL1(−/−) cells in the ranking mode was carried out by lipid ontology (LION): a web-based interface used for the identification of lipid-associated terms in lipidomes (189). The cut-off value of significantly up and down lipids in HEK293 MUL1(−/−) with respect to WT cells (P<0.05) was determined and the data was scaled with the enrichment (−log FDR q-values).

### RNA Sequencing Analysis

Standard RNA-sequencing for gene profiling expression of protein-coding sequences (mRNA) was performed using HEK293 WT and MUL1(−/−) cells. Approximately 10 million HEK293 WT and MUL1(−/−) cells (n=3 per group) were used for library preparation, DNA sequencing, and data analysis (GENEWIZ Global). Using DESeq2, a comparison of gene expression between the defined groups of samples was performed. The Wald test was used to generate P-values and log 2 fold changes. Genes with an adjusted P value <0.05 (P<0.05) and absolute log 2 fold change >1 were labelled as differentially expressed genes. Significantly differentially expressed genes were clustered by their gene ontology and the enrichment of gene ontology terms was tested using Fisher exact test. Heatmaps of the top fifty, as well as, genes specific involved in lipid, glucose, carbohydrate, glutathione and retinoic acid metabolomic processes, were analyzed using RStudio and Heatmap.2 software (190).

### Statistical Analysis

All quantitative data were expressed as mean±SD, or ±SEM of three or four independent experiments. Following Western blot analysis, the optical densities of blot bands were determined using ImageJ software. Protein/β-actin ratios were obtained from the densitometry data, and the differences among groups were analyzed by one tailed Student's t test. A value of P≤0.05 was considered significant. All Seahorse data were analyzed using Report Generators software that automatically calculates and reports the assay parameters of the Agilent Seahorse XFe24 specific for each assay (Glycolysis Stress Test or Cell Mito Stress) (Agilent Technologies). For data analysis (NMR spectroscopy, LC-MS metabolomic, lipidomic as well as RNA-seq) a value of P≤0.05 was considered significant.

### Results-Study One

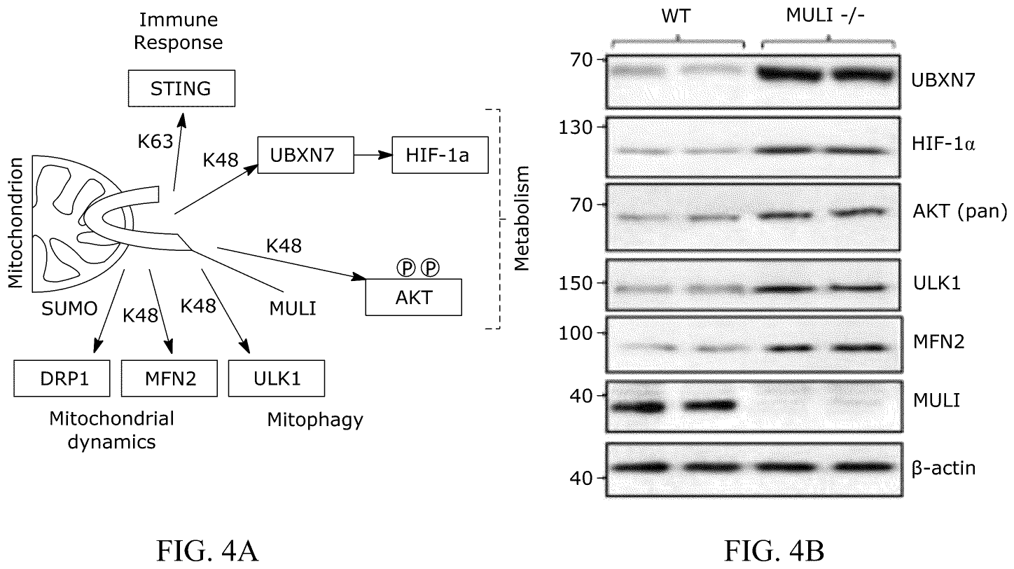

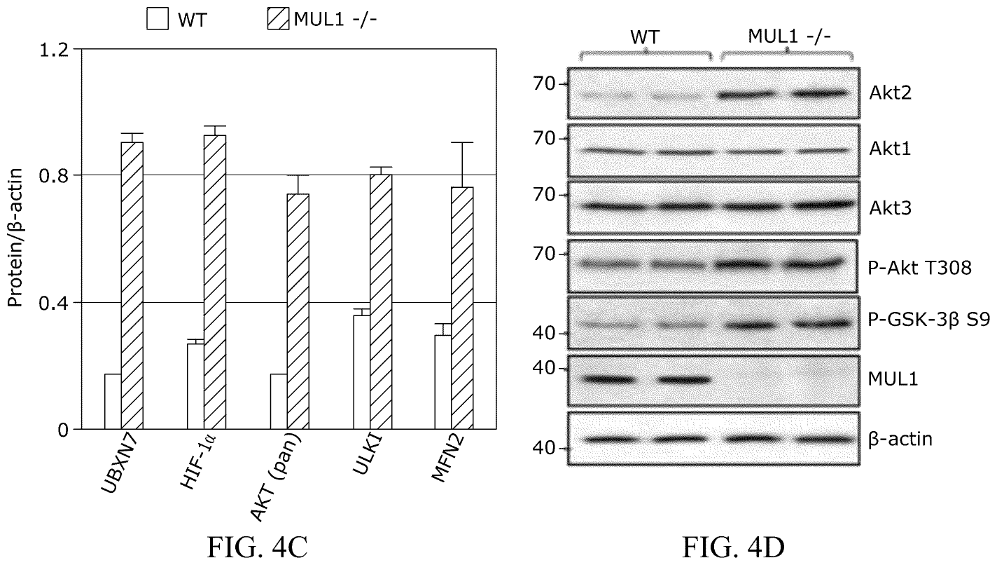

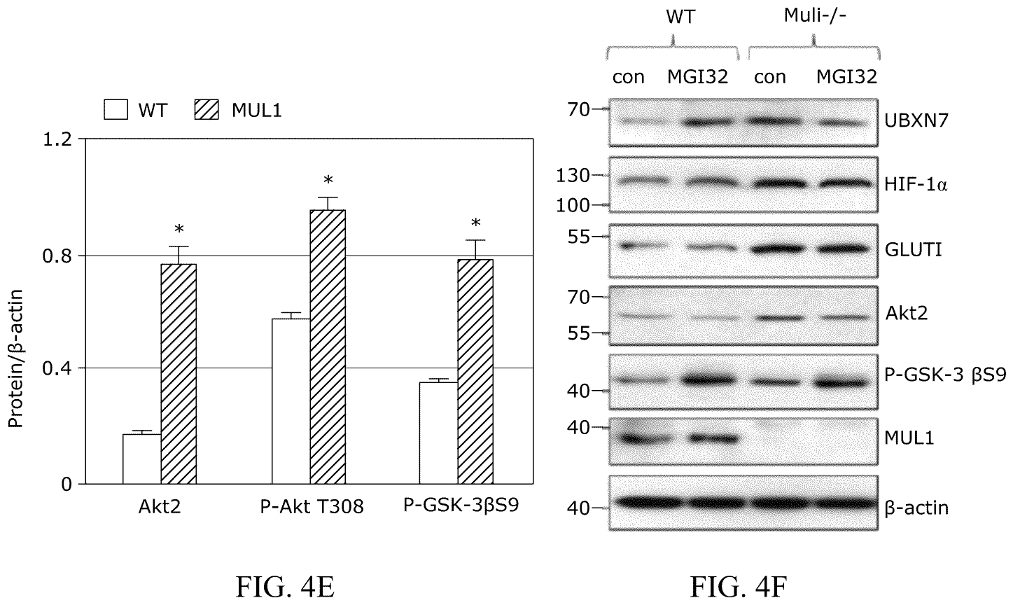

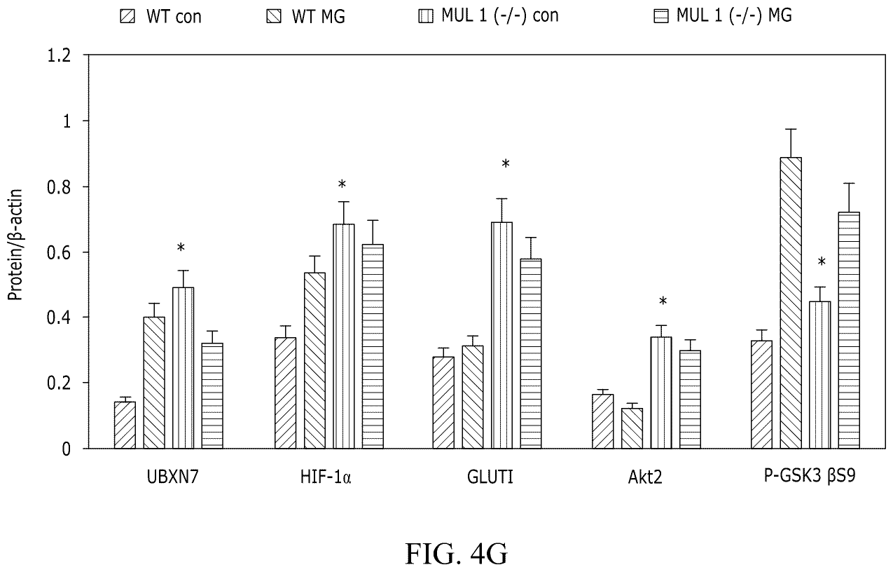

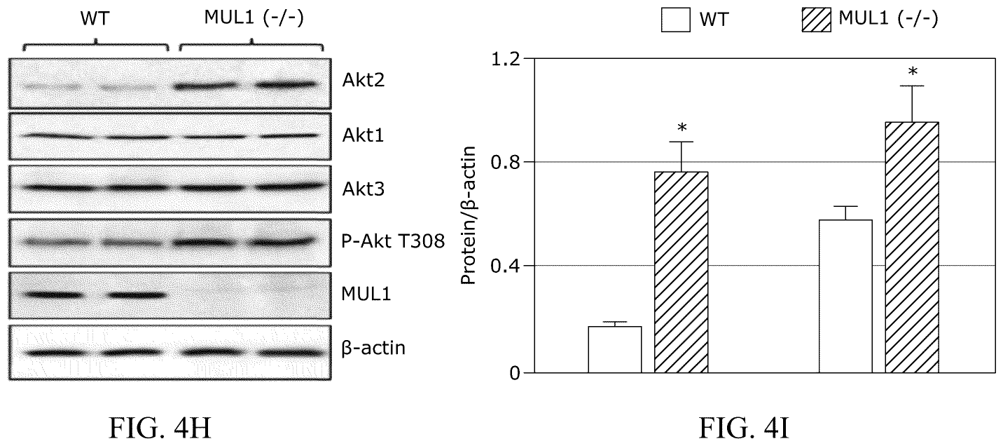

MUL1 E3 ubiquitin ligase regulates the protein level of several substrates with diverse function. MUL1-mediated K48-polyubiquitination invariably targets its substrates for proteasomal degradation and therefore regulates their protein level. There are four such known substrates: MFN2, ULK1, AKT, as well as the UBXN7 protein that controls the protein level of HIF-1α (FIG. 4A). (6; 28; 59; 37; 39; 22). FIG. 4B shows the protein level of UBXN7, HIF-1α, AKT, ULK1, and MFN2 in two independent clones of HEK293 WT or MUL1(−/−), where MUL1 has been inactivated using CRISPR-Cas9 (152; 159; 209; 22). The absence of MUL1 ligase leads to significant accumulation of all these substrate proteins (FIG. 4C). AKT defines a family of three different but highly homologous kinases, Akt1, Akt2, and Akt3 (137). To determine which one of them is regulated by MUL1, their respective protein level in HEK293 MUL1(−/−) cells using specific antibodies was monitored. FIG. 4D shows that Akt2 alone is regulated in the absence of MUL1, and there was no detectable change in either Akt1 or Akt3 protein level. In addition, accumulation of Akt2 in HEK293 MUL1(−/−) cells leads to its activation as seen by the degree of autophosphorylation (P-Akt2 Thr308) as well as the increased phosphorylation of its GSK-313 S9 substrate (FIGS. 4D-E) (141; 143).

Additionally, to verify that the results were not restricted to HEK293 MUL1(−/−) cells alone, a HeLa MUL1(−/−) cell line was created. FIGS. 4F-G show that in HeLa MUL1(−/−) cells, UBXN7, HIF-1α, and Akt2 protein levels, as well as their activation reflected in the upregulation of GLUT1 protein and GSK-313 S9 phosphorylation, closely mirror the result observed in HEK293 MUL1(−/−) cells.

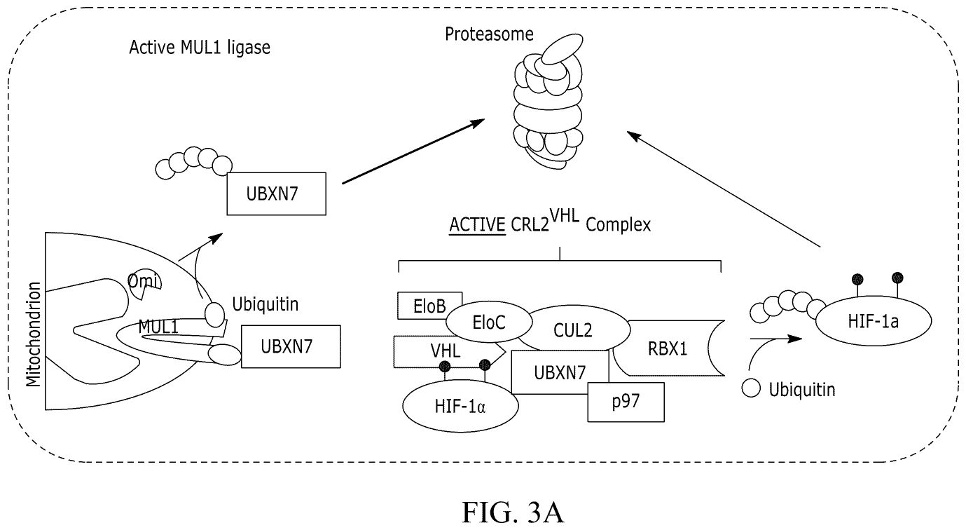

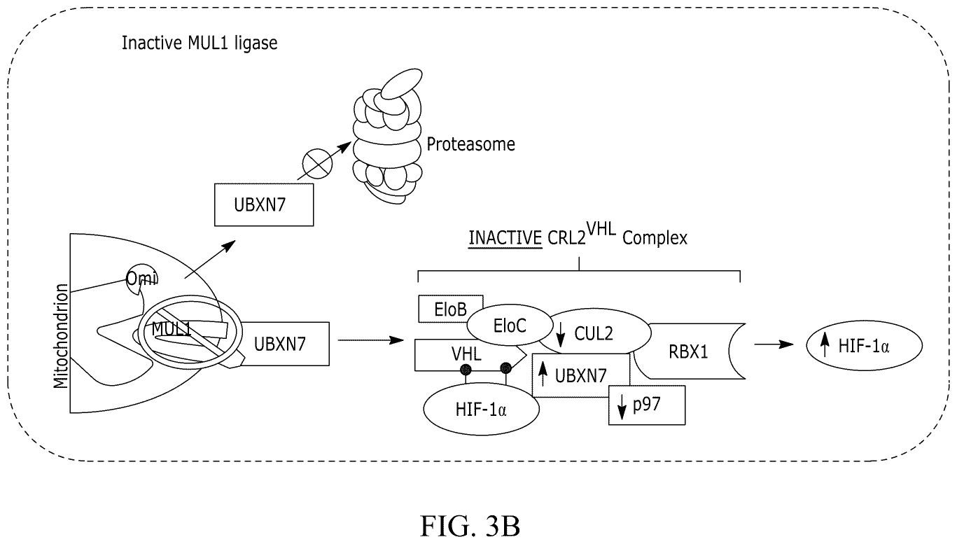

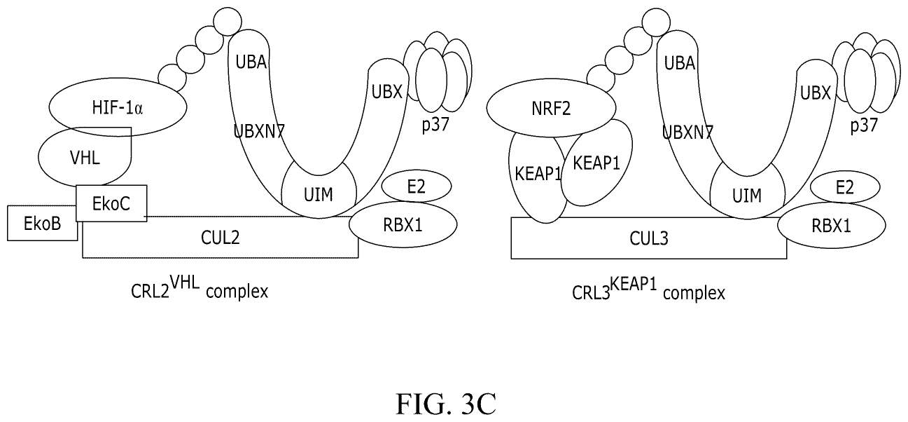

Furthermore, the instant inventors identified a new substrate of MUL1, the UBXN7 cofactor and scaffold protein of the CRL3^{KEAP1} and CRL2^{VHL} complexes (FIG. 3C) (34). It was found that inactivation of MUL1 leads to accumulation of UBXN7 which modulates the CRL2^{VHL} complex to increase the stability and activate HIF-1α in the absence of hypoxia (22). During the same conditions, accumulation of UBXN7 suppress the NRF2 transcription factor that regulated the CRL3^{KEAP1} complex. The role of this reciprocal regulation of HIF-la and NRF2 by UBXN7 in the metabolic function of MUL1 is indeterminate. In addition, MUL1 through K48-ubiquitination regulates the protein level of Akt2 kinase alone but has no effect on the other two members of the AKT family, Akt1 and Akt3 (FIGS. 4H-I). The coactivation of Akt2 and HIF-1α that occurs in MUL1(−/−) cells has never been observed or studied before.

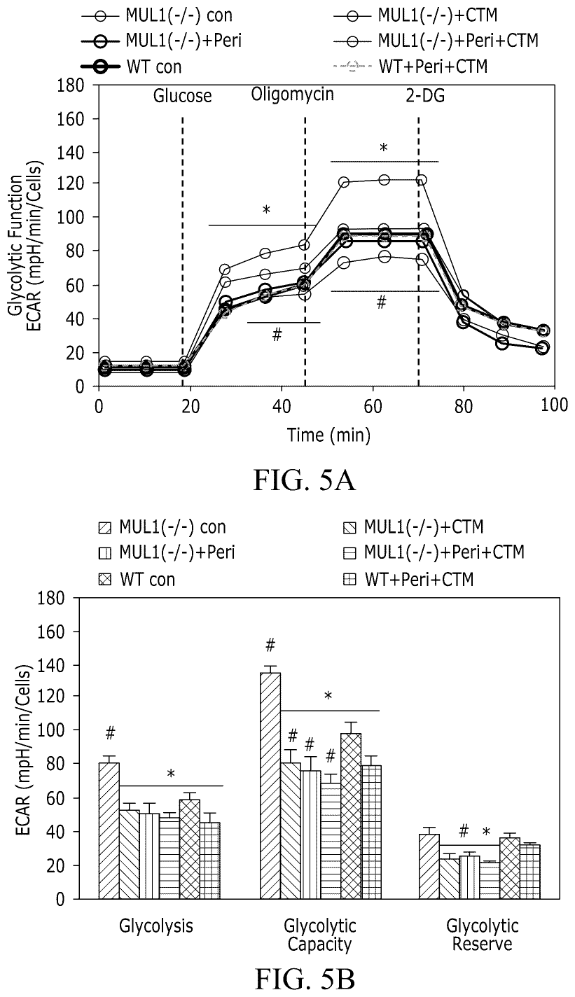

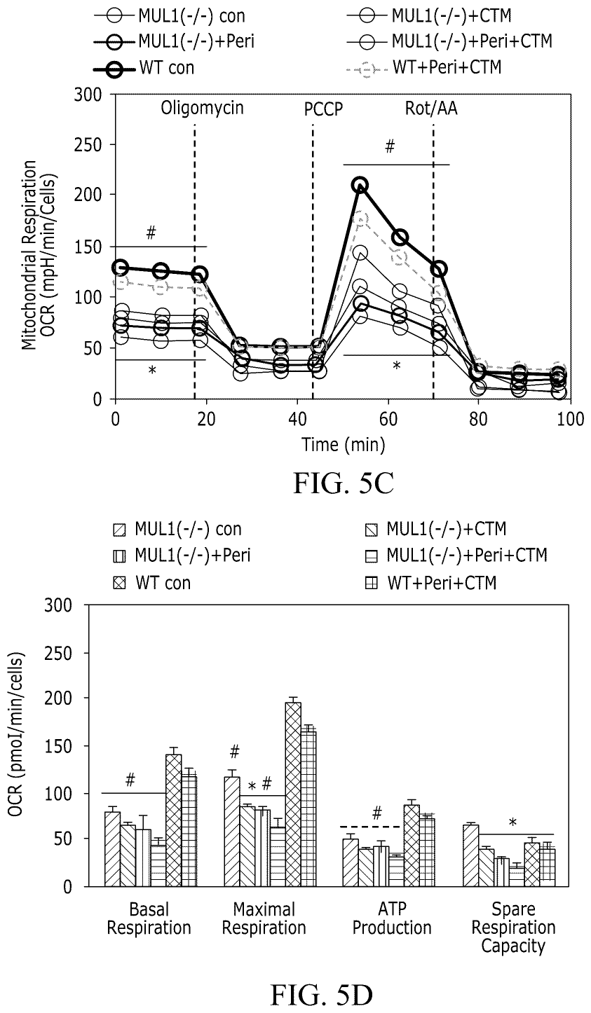

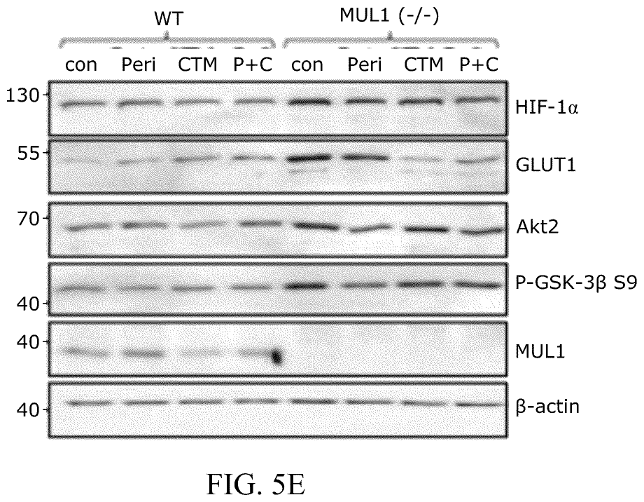

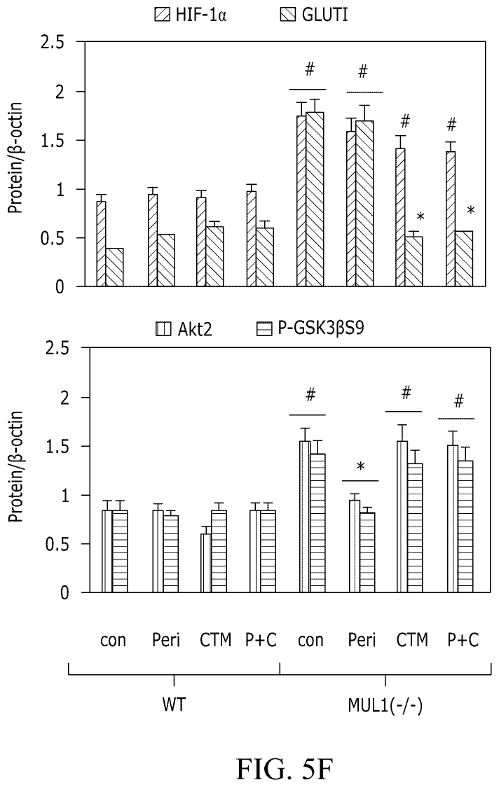

Contribution of HIF-1α and Akt2 to mitochondrial respiration and the glycolytic phenotype of HEK293 MUL1(−/−) cells. The Glycolytic Stress test was performed using a Seahorse XF^{e}24 analyzer. Glycolysis, glycolytic capacity as well as glycolytic reserve in HEK293 WT and MUL1(−/−) cells was monitored. The individual involvement of HIF-la and/or Akt2 proteins, in this process, were investigated using the specific HIF-1α inhibitor, chetomin, or the Akt2 inhibitor, perifosine (140; 158; 109; 142). FIG. 5A compares the glycolytic function, by real-time measurement of the extracellular acidification rate (ECAR) in HEK293 WT and MUL1(−/−) cells. Treatment of HEK293 MUL1(−/−) cells with perifosine reduced ECAR and glycolysis to a level lower than the one present in HEK293 WT cells (FIGS. 5A-B). Chetomin alone or in combination with perifosine works as a very potent inhibitor of the mitochondrial glycolytic function (FIG. 5A). These results clearly demonstrate that both Akt2 and HIF-1α proteins work synergistically and are indispensable for the glycolytic phenotype observed in HEK293 MUL1(−/−) cells. FIG. 5B represents the quantification of the measurements of glycolysis, glycolytic capacity as well as the glycolytic reserve in cells untreated or treated with HIF-1α and Akt2 inhibitors. A mitochondrial respiration assay was also performed and FIG. 5C is a trace of the oxygen consumption rate (OCR) of HEK293 WT and MUL1(−/−) cells untreated or treated with perifosine and/or chetomin. The OCR of HEK293 MUL1(−/−) is lower compared to the HEK293 WT cells and is further reduced after treatment with perifosine, chetomin, or with both inhibitors. FIG. 5D is a quantitative assessment of the data comparing the basal, maximal respiration, ATP production, and spare respiratory capacity. In addition, perifosine or chetomin at the concentration used here, are potent inhibitors of mitochondrial respiration and they are very effective against their respective targets of HIF-1α and Akt2 (FIG. 5E). Chetomin significantly reduces the expression of GLUT1, and perifosine inhibits Akt2 kinase activity (FIGS. 5E-F).

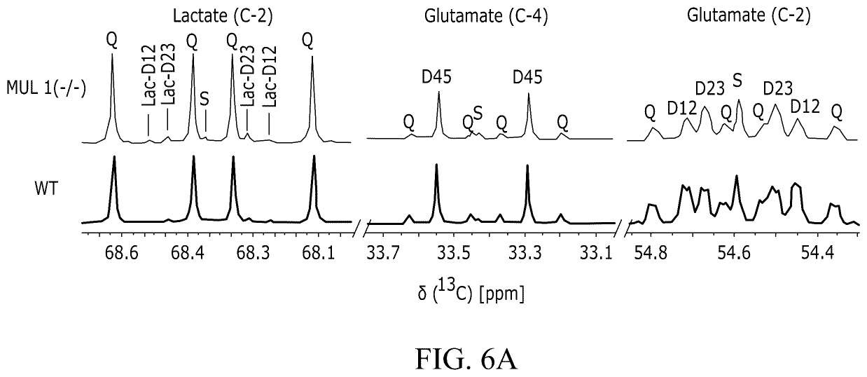

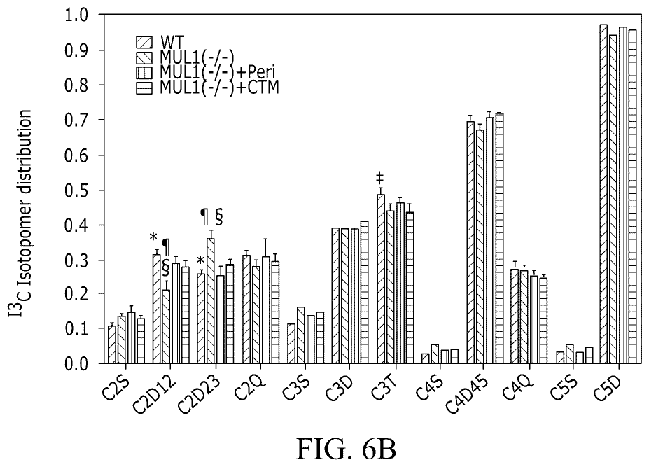

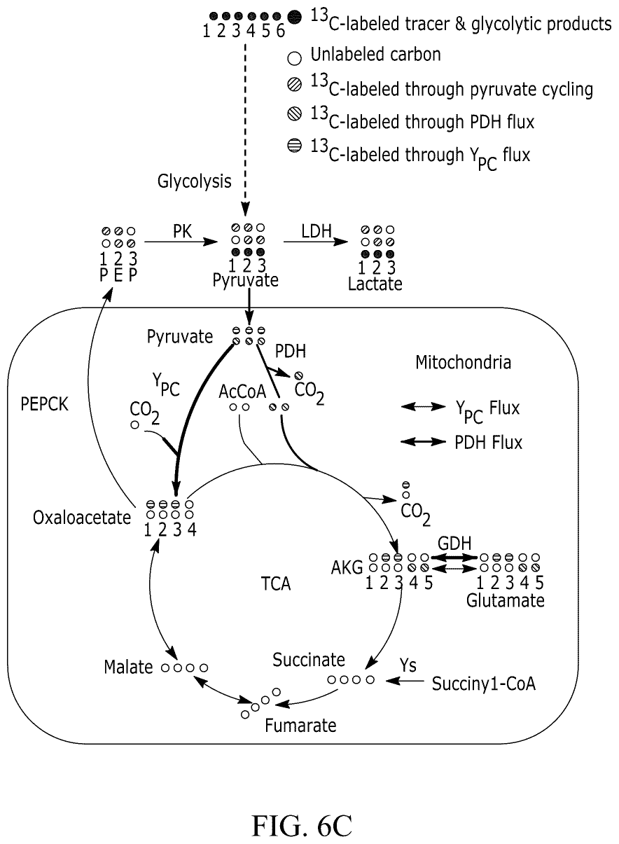

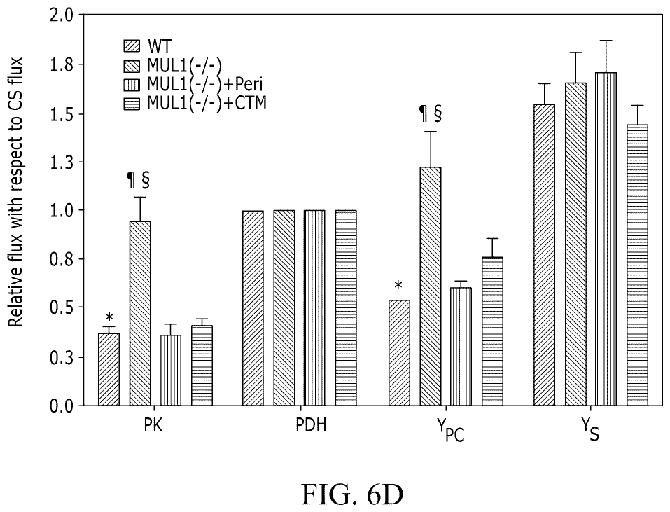

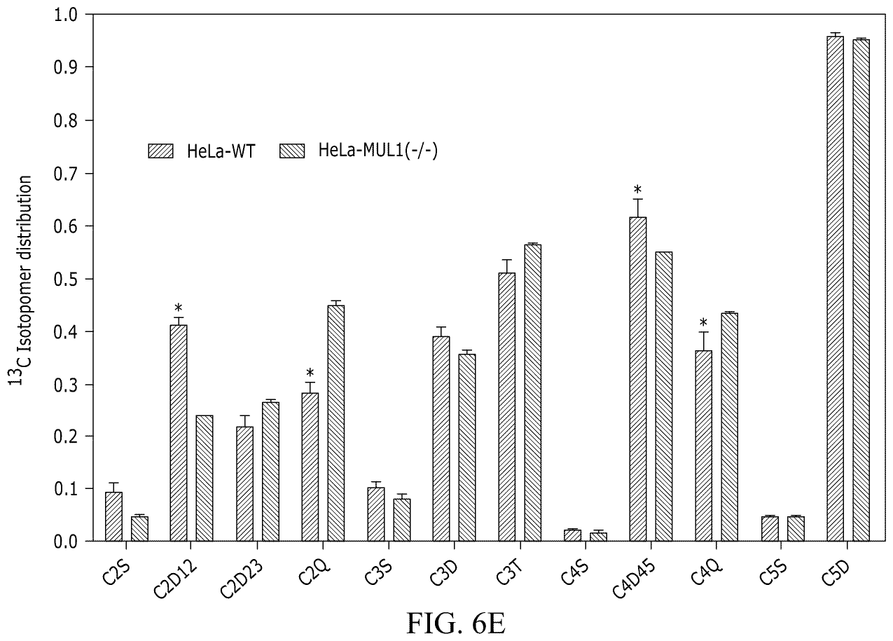

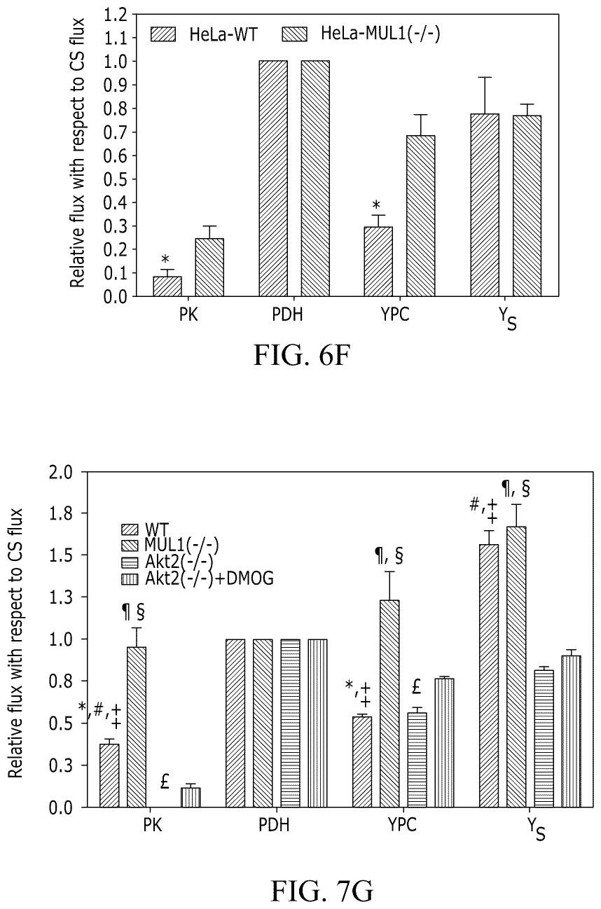

Regulation of the metabolic flux in HEK293 cells. The effects of MUL1 inactivation and the role of HIF-1α and Akt2 accumulation on the metabolic flux using NMR-based ¹³C-isotopomer analysis was investigated. HEK293 MUL1(−/−) treated with Akt2 or HIF-la inhibitors and WT cells were labelled using a [U-¹³C]glucose tracer in the cell culture medium. After multiple turns of the TCA cycle, all carbon positions of the glutamate were labeled, resulting in a complex but well-defined pattern of ¹³C-NMR spectra (153; 139). A representative ¹³C-NMR spectrum of glutamate is shown in FIG. 6A demonstrating the ¹³C-labeling pattern of C-2 and C-4 carbons. The results of the glutamate ¹³C signal ratio obtained from ¹³C-NMR spectra of HEK293 WT, MUL1(−/−), MUL1(−/−)+perifosine, and MUL1(−/−)+chetomin are summarized in FIG. 6B. The schematic diagram of the metabolic flux model in FIG. 6C shows the ¹³C-isotope labeling patterns of the metabolites derived from [U-¹³C]glucose metabolism after a single turn of the cycle. [U-¹³C]glucose derived [U-¹³C]pyruvate produces lactate via LDH and can enter the tricarboxylic acid (TCA) cycle through Y_{PC} (red arrow) and PDH flux (green arrow). In the first turn of the TCA cycle, [U-¹³C]pyruvate oxidation through PDH produces labeled glutamate C4-C5 (green dots) positions, whereas Y_{PC} flux labels glutamate C2-C3 (red dots). Pyruvate cycling via phosphoenolpyruvate carboxykinase (PEPCK) and PK fluxes is also shown in FIG. 6C. Pyruvate cycling was included as a possible pathway, as the presence of [1,2-¹³C₂] and [2,3-¹³C₂]lactate isotopomers is only possible via this pathway (FIG. 6A). The relative metabolic flux rates of PK, Y_{PC}, pyruvate dehydrogenase (PDH), and anaplerosis leading to succinyl-CoA (Yₛ) were estimated, using glutamate ¹³C-isotopomer distribution data obtained from ¹³C-NMR analysis of the cell extracts. All flux rates are referenced to a citrate synthase (CS) flux of 1 and is equivalent to Kreb's cycle flux. Using tcaCALC, multiple different metabolic models can be compared to each other, with the most parsimonious model selected (FIG. 6D) (128). HEK293 MUL1(−/−) cells significantly increased the PK and Y_{PC} fluxes compared to WT cells and the Akt2 or HIF-1α inhibitors significantly decreased the PK and Y_{PC} flux rates in MUL1(−/−) cells compared to untreated cells (FIG. 6D).

Additionally, the same metabolic flux analysis using the [U-13C]glucose tracer for HeLa WT and HeLa MUL1(−/−) cells to confirm the effect of MUL1 knock-out on energy metabolism in an alternative cell line was performed. Glutamate isotopomer analysis of HeLa WT and MUL1(−/−) cells show the same effects of increased pyruvate anaplerosis into the TCA cycle as well as significant flux through PK (FIGS. 6E-F). These results closely mirror those seen in the HEK293 cells.

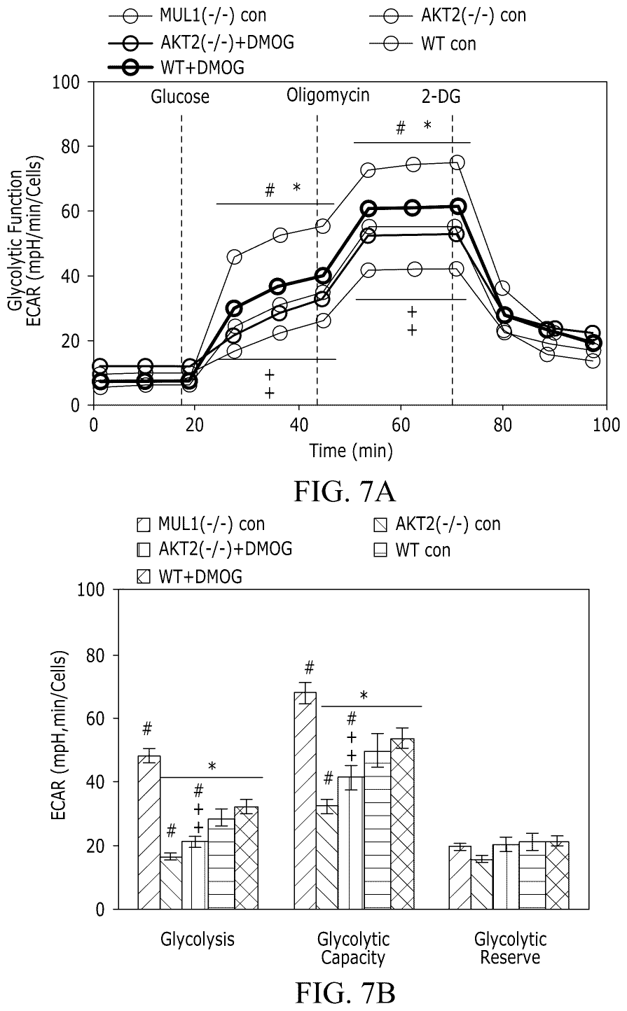

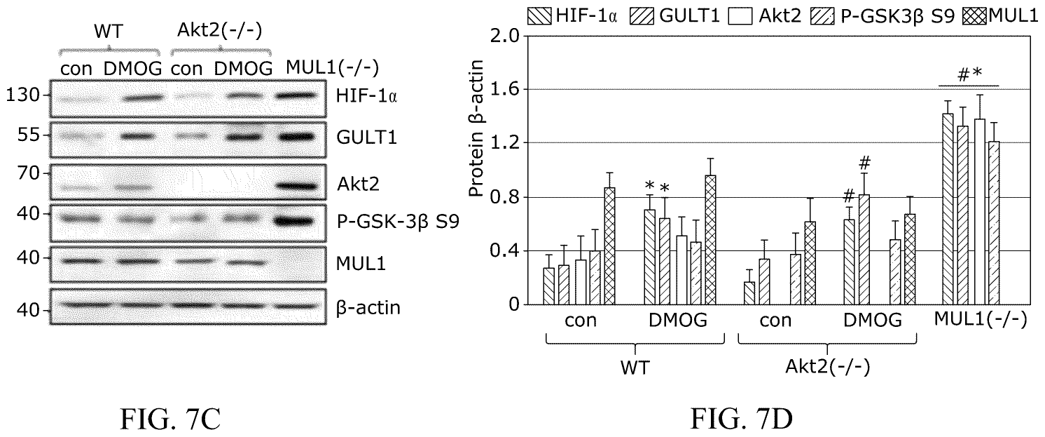

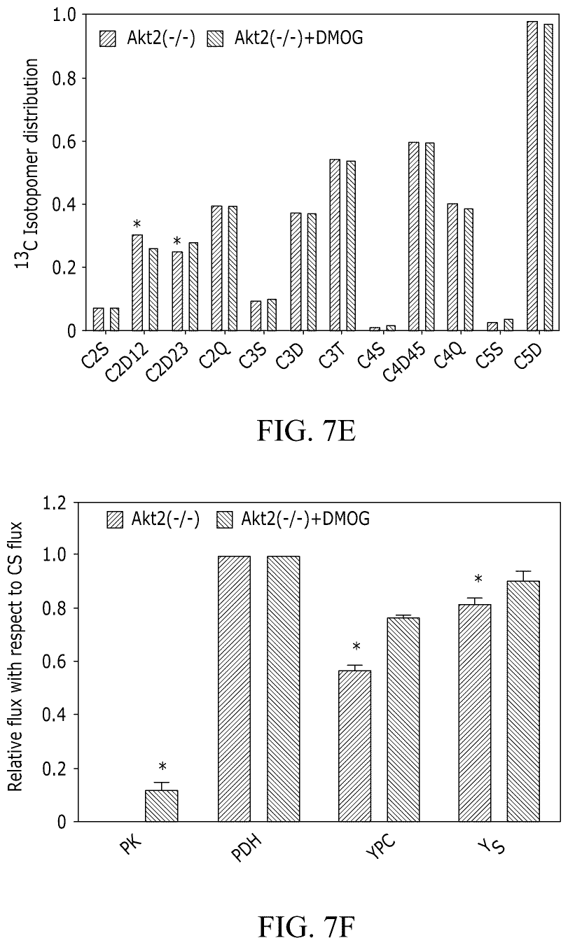

Effect of HIF-1α activation in the regulation of glycolysis in HEK293 Akt2(−/−). HEK293 Akt2(−/−) cells using CRISPR-Cas9 (see Methods section) were created in order to investigate the role of HIF-1α in glycolysis in the absence of the Akt2 protein. ECAR was measured for both HEK293 Akt2(−/−) and WT cells in the presence or absence of dimethyloxalyglycine (DMOG), a known activator of HIF-1α (162). HEK293 Akt2(−/−) had substantially reduced glycolysis compared to MUL1(−/−) cells (FIG. 7A). When HIF-1α was activated in HEK293 Akt2(−/−) cells, there was a small but significant increase in glycolysis, as well as glycolytic capacity (FIG. 7B). The same treatment in HEK293 WT cells was unremarkable (FIGS. 7A-B). FIG. 7C is Western blot analysis to monitor the protein expression and activity of HIF-1α and Akt2 proteins in HEK293 WT and Akt2(−/−) cells before or after treatment with DMOG. FIG. 7D is a densitometric analysis of the Western blot data from FIG. 7C normalized against β-actin. The combined data from these experiments strongly suggest that accumulation and activation of both Akt2 and HIF-1α is required for the glycolytic phenotype observed in MUL1(−/−) cells. Furthermore, we performed a ¹³C-NMR-based metabolic flux analysis to determine the effect of HIF-1α on metabolism in HEK293 cells by HIF-1α activation in the absence of Akt2 protein. The isotopomer results from ¹³C-NMR spectra of HEK293 Akt2(−/−) and Akt2(−/−)+DMOG cells are summarized in FIG. 7E. ¹³C-labeling pattern in glutamate for C2D12 and C2D23 multiplets were significantly different between HEK293 Akt2(−/−) and Akt2(−/−)+DMOG cells (FIG. 7E). Metabolic flux analysis results suggested that the activation of HIF-1α by DMOG significantly increased PK, Y_{PC}, and Y_{S} fluxes (FIG. 7F).

The metabolic fluxes of Akt2(−/−) cells with or without DMOG were also compared with HEK293 WT and MUL1(−/−) cells and plotted in FIG. 7G. Akt2 knockout significantly downregulates PK flux compared to HEK293 WT cells. PK flux was partially restored with DMOG, but did not reach the WT levels, while Y_{PC} was elevated above WT, but fell short of reaching MUL1(−/−) levels (FIG. 7G). Anaplerosis via succinyl-CoA (Yₛ) was significantly lower in both Akt2(−/−) and Akt2(−/−)+DMOG cells compared to WT and MUL1(−/−) cells (FIG. 7G). All flux rates are referenced to a citrate synthase (CS) flux of 1 and is equivalent to Kreb's cycle flux.

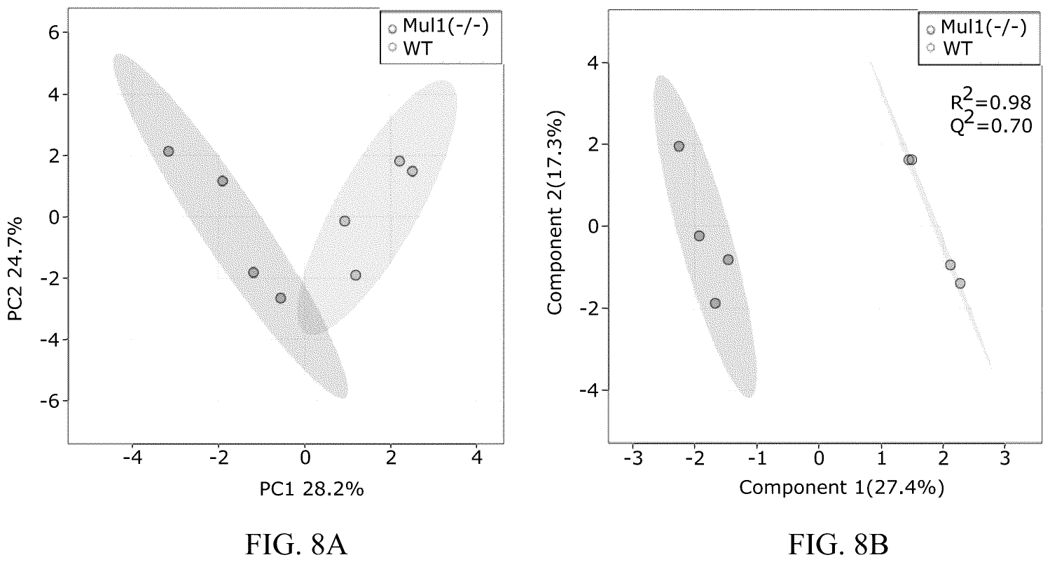

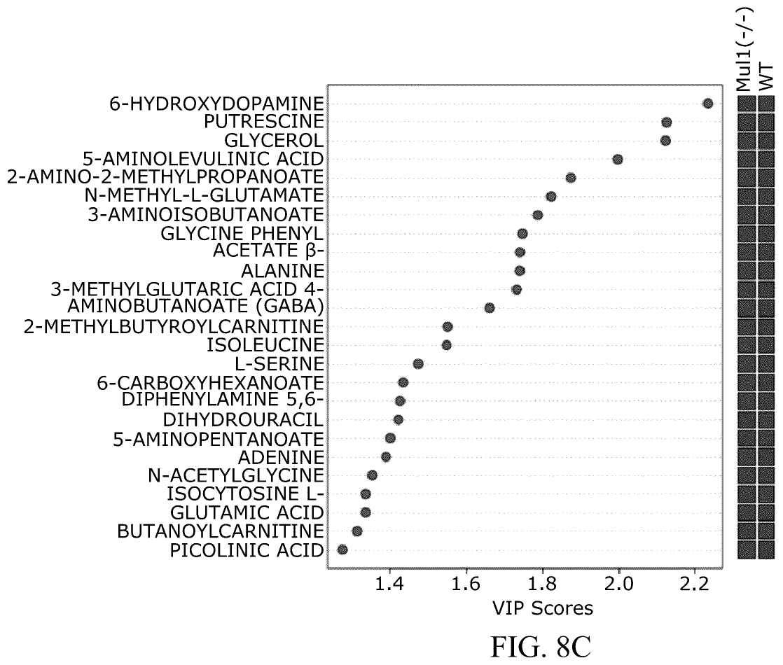

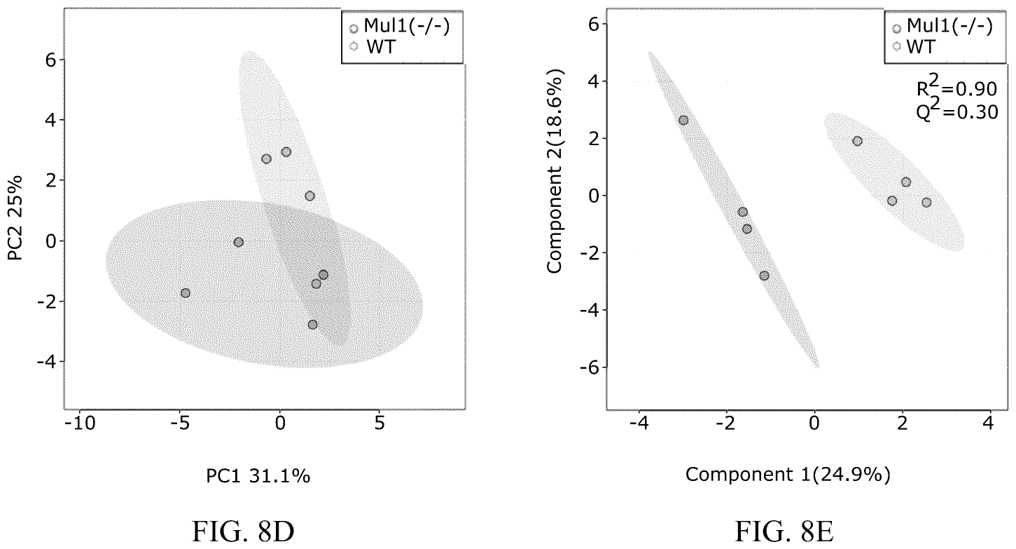

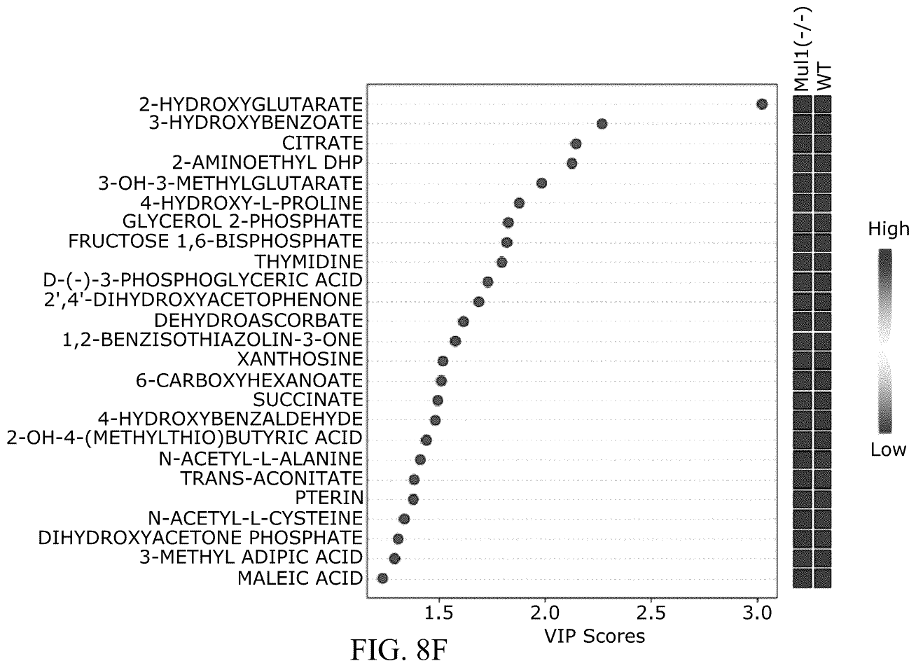

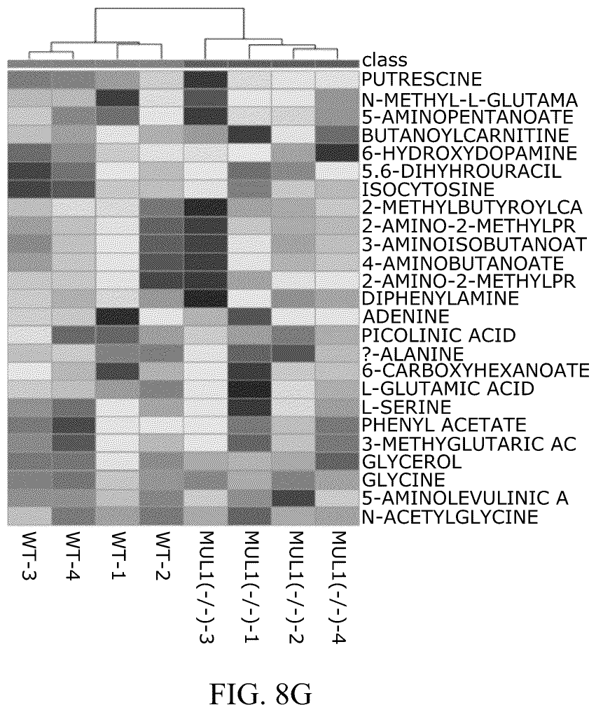

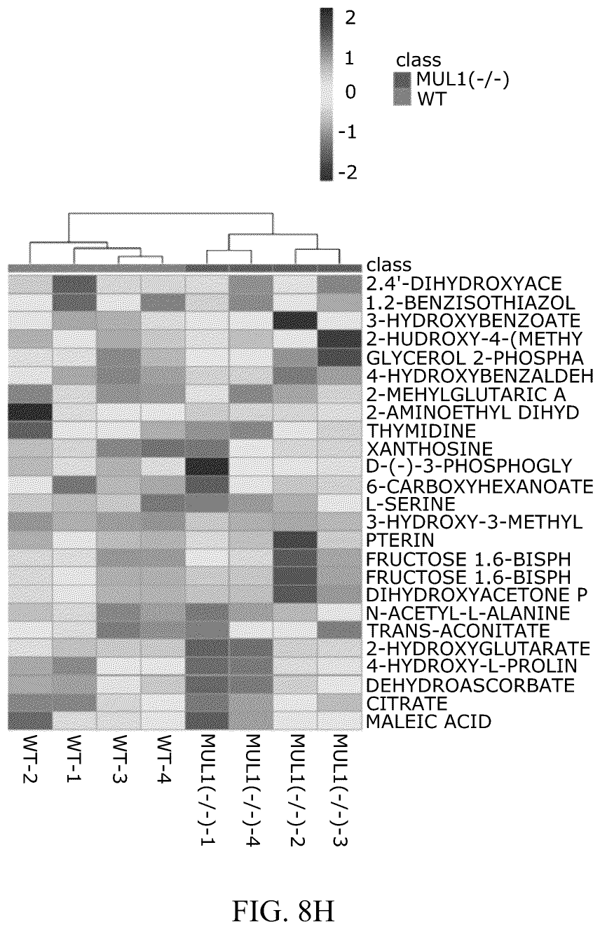

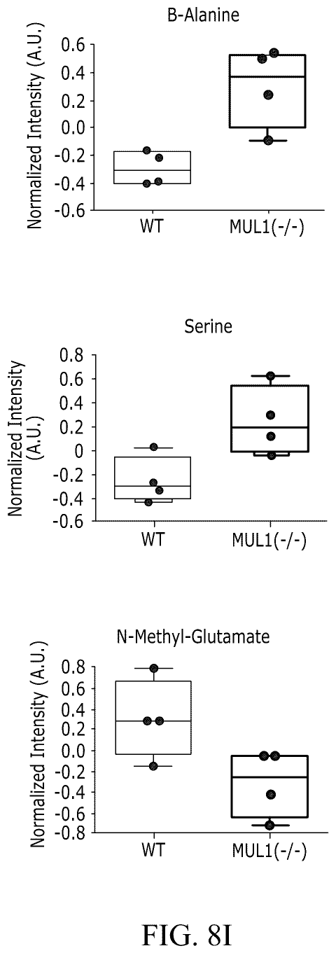

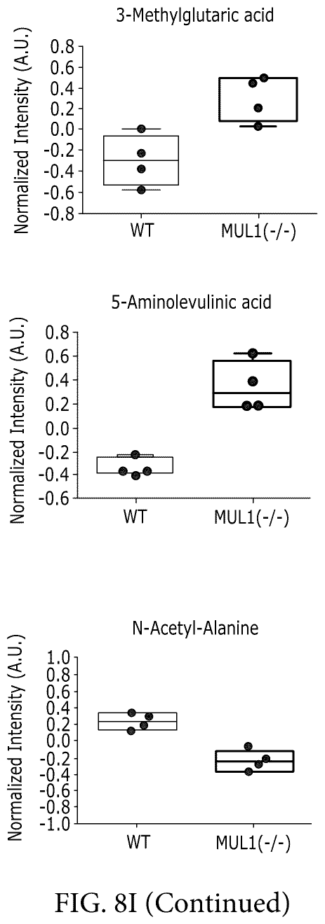

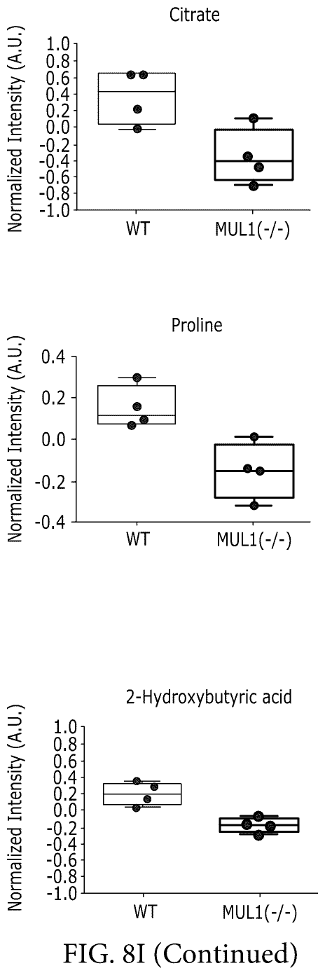

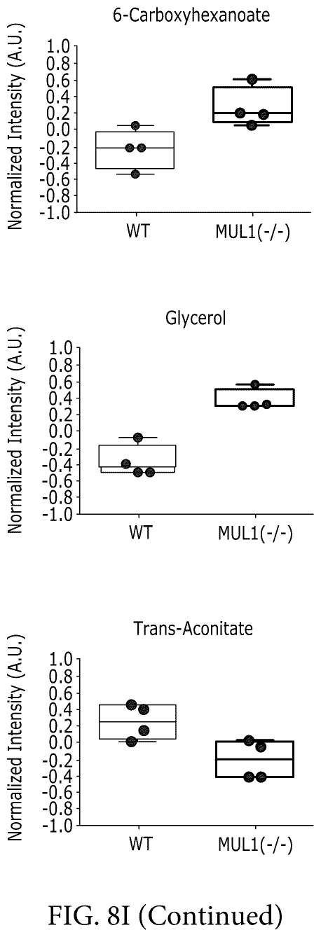

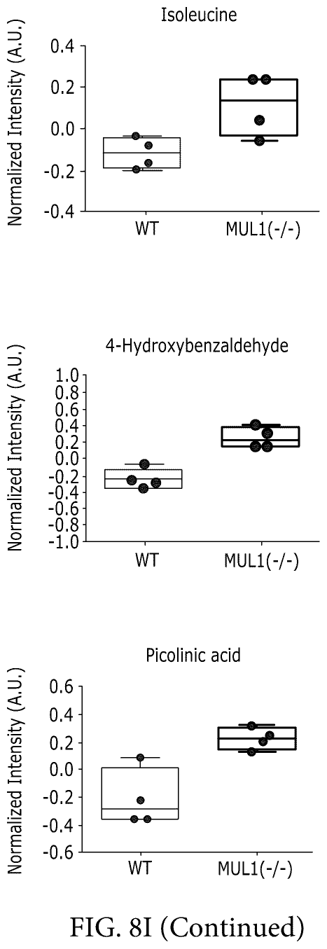

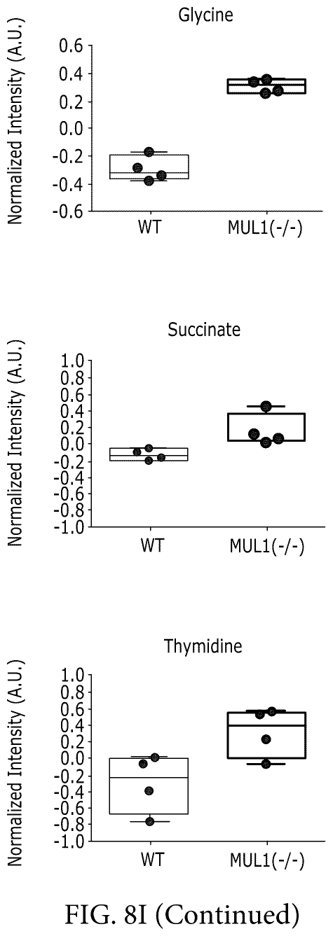

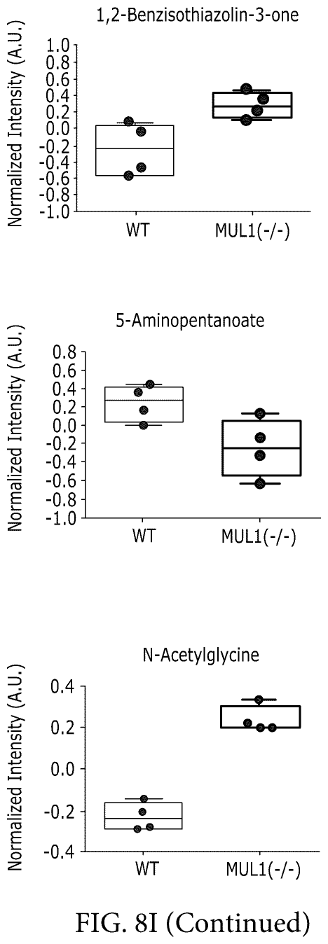

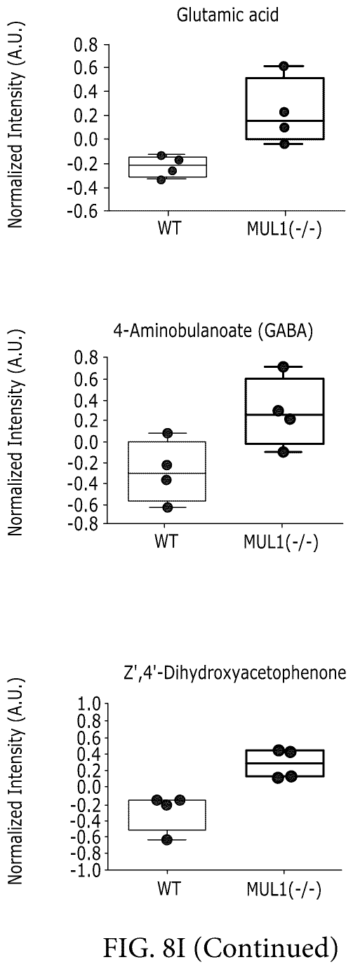

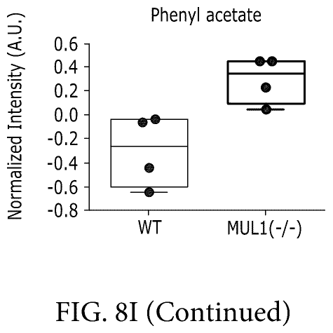

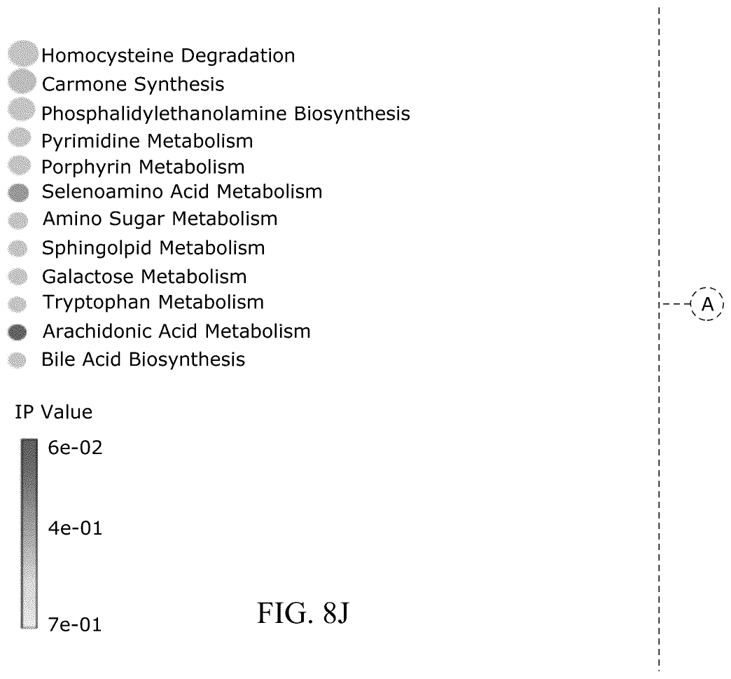

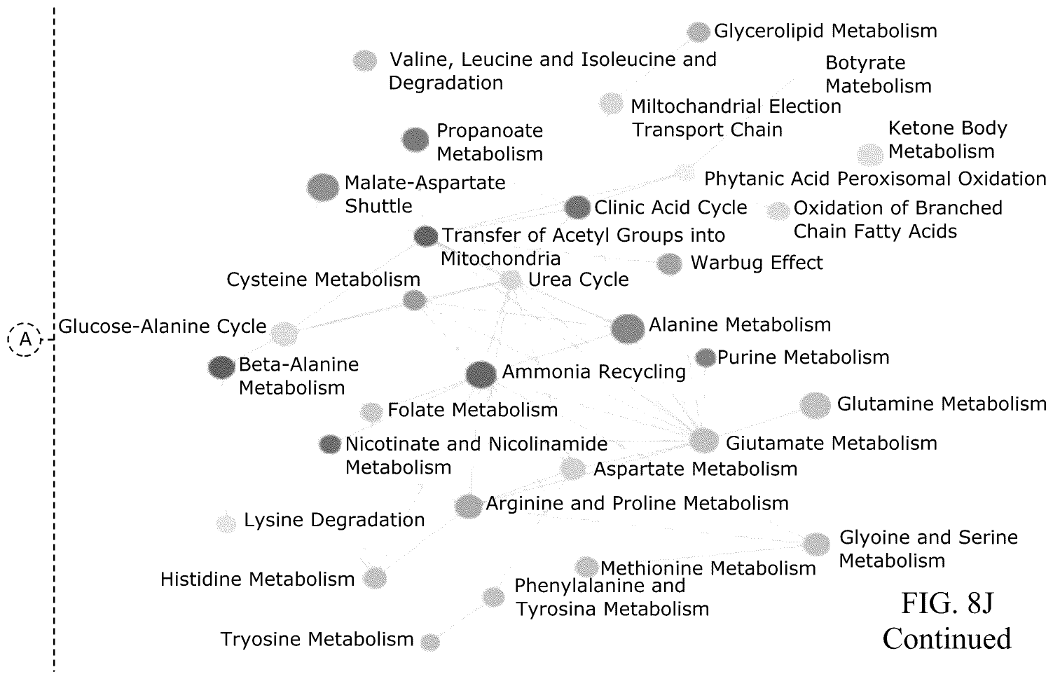

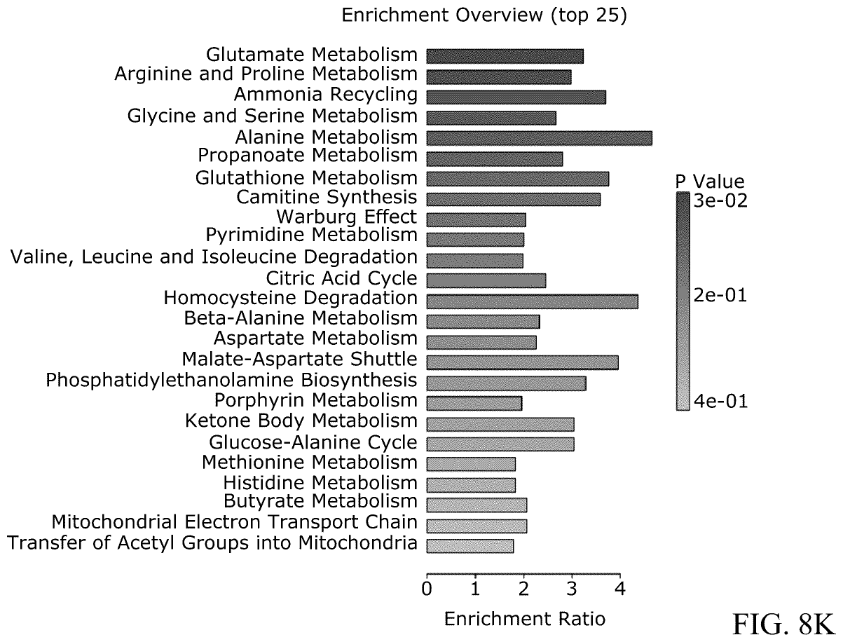

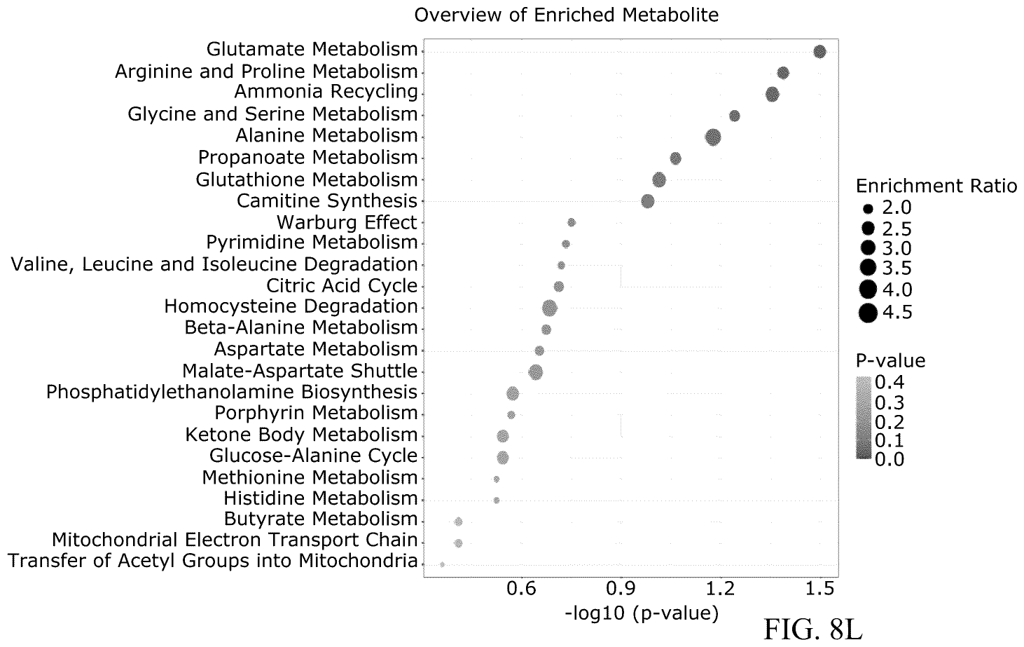

The role of MUL1 in the metabolic homeostasis. Detailed global metabolic studies using HEK293 MUL1(−/−) and WT cells was performed. The unsupervised principal component analysis (PCA) 2D scores plots showed clear separation in the metabolic profile between HEK293 WT and MUL1(−/−) cells in LC-MS positive ion mode (FIG. 8A). Whereas, for LC-MS negative ion mode data, partial separation was achieved between both groups in PCA (FIG. 8D). The supervised partial least squares discriminant analysis (PLS-DA) model was used to predict the classes of samples and maximize the separation between groups (FIGS. 8B and 8E). To determine the robustness of the mathematical model, R² and Q² parameter values were calculated for the PLS-DA model. The R²/Q² values for LC-MS positive and negative ion modes data were 0.98/0.70, and 0.90/0.30, respectively (FIGS. 8B and 8E). The PLS-DA variable importance projection (VIP) scores plot from LC-MS positive and negative modes PLS-DA model identified a large group of metabolites different between HEK293 WT and MUL1(−/−) cells (FIGS. 8C and 8F).

Heatmaps of the top 25 metabolites from both positive and negative mode MS demonstrated excellent clustering of the samples based on MUL1 status (FIGS. 8G-H). Significantly different metabolites were identified by either PLS-DA or T-test, class membership for each sample. The semi-quantitative metabolomics panel from LC-MS positive and negative modes data in FIG. 8I summarized the significantly different metabolites between HEK293 WT and MUL1(−/−) cells. The metabolites such as β-alanine, glycerol, glycine, N-acetylglycine, serine, succinate, glutamic acid, isoleucine, thymidine, GABA, and picolinic acid were found significantly higher in HEK293 MUL1(−/−) cells. Whereas, N-acetyl-alanine, citrate, trans-aconitate, 3-methylglutaric acid, proline, 2-hydroxybutyric acid, 5-amino pentanoate were significantly higher in HEK293 WT cells (FIG. 8I). All significantly different metabolites between HEK293 WT and MUL1(−/−) cells were utilized for Metabolite Set Enrichment Analysis (MSEA) to produce a network of pathways. MSEA results indicate that the provided metabolites set has significantly enriched the glutamate, arginine, proline, and ammonia recycling metabolic pathways. In addition, glycine, serine, alanine, and propanoate metabolic pathways were also substantially enhanced in the absence of MUL1 (FIGS. 8J-L).

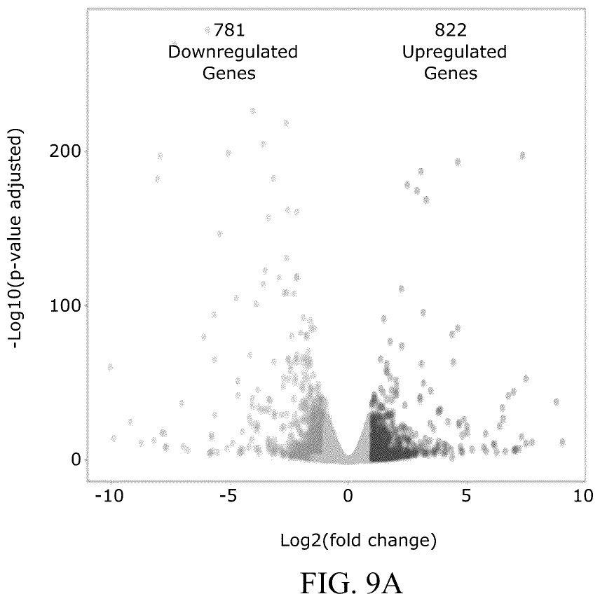

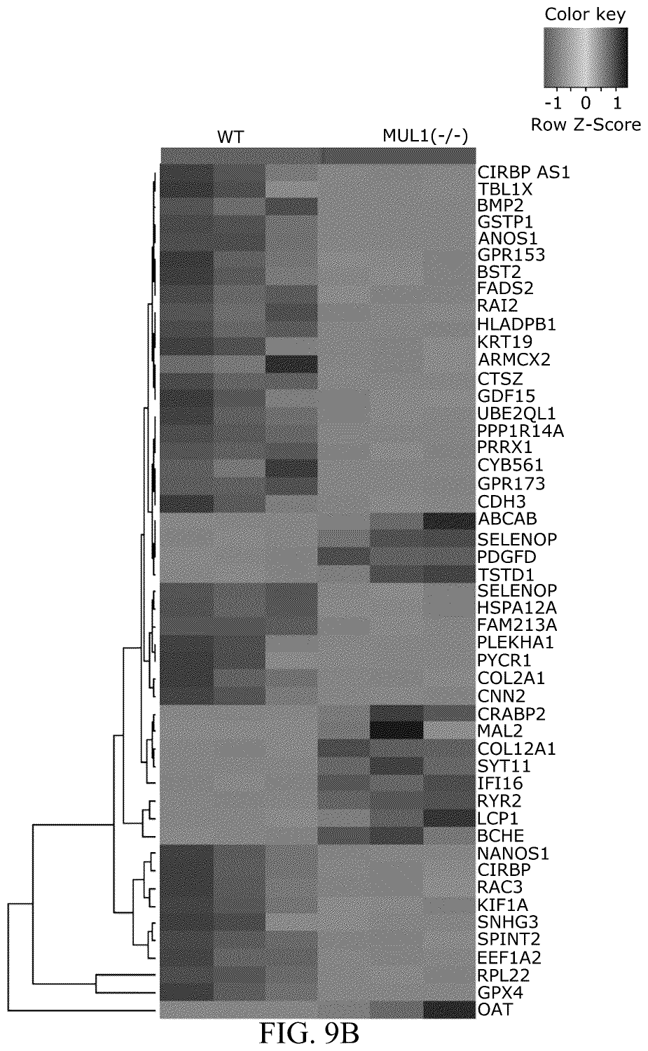

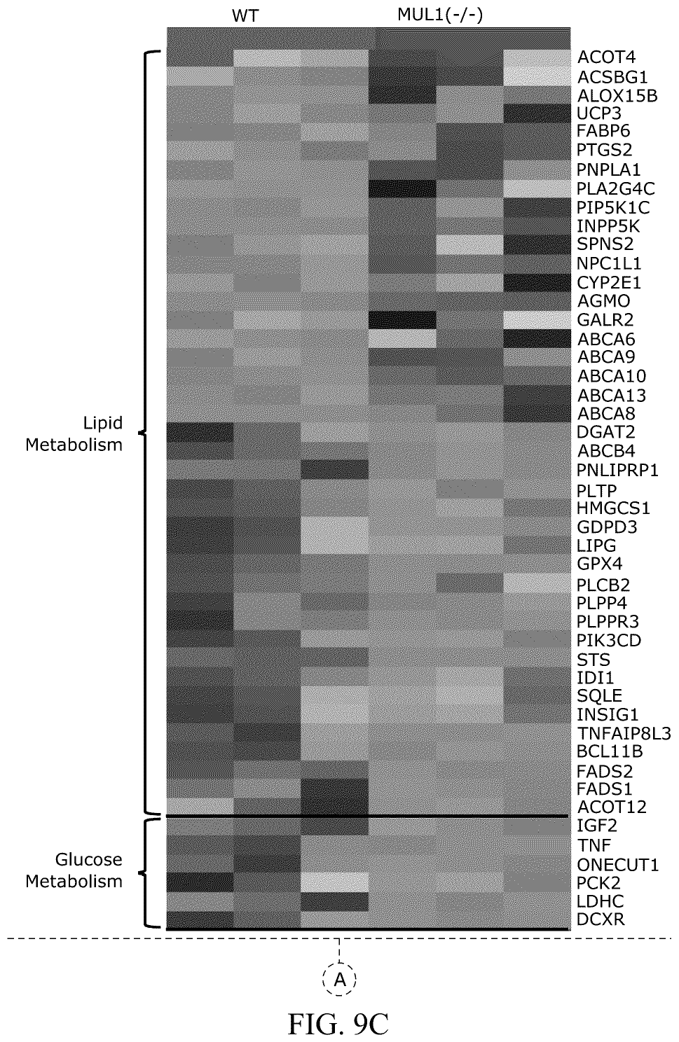

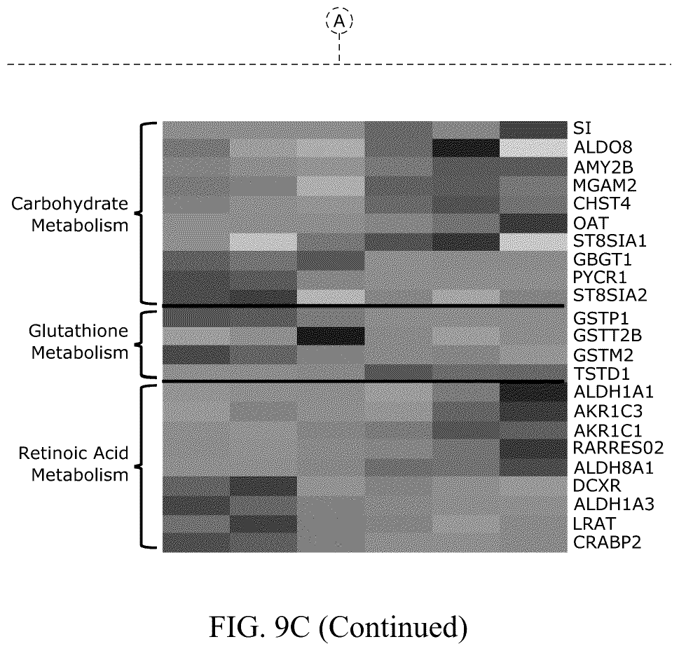

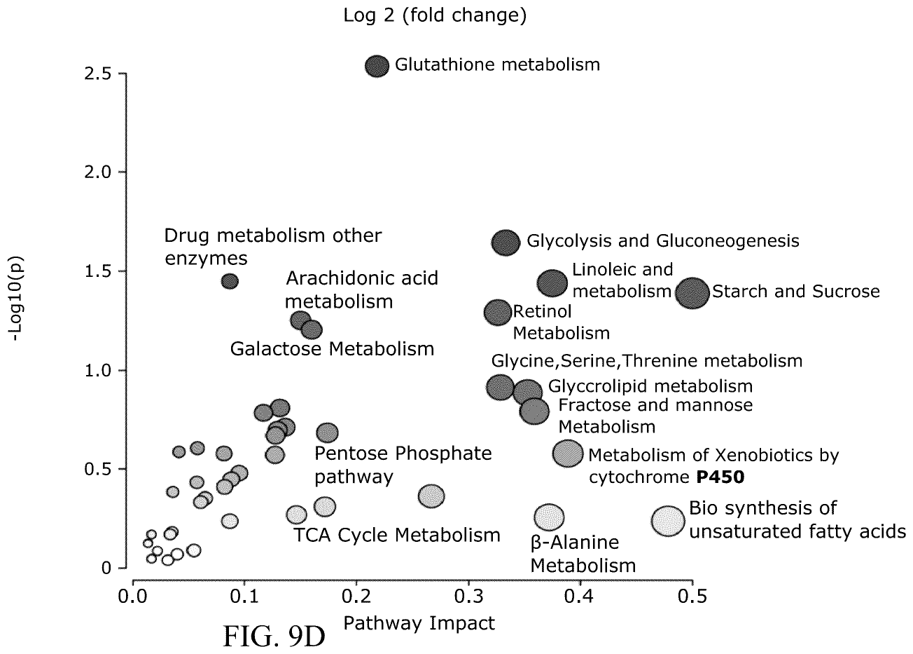

Alterations in global gene expression and Integrated multi-omics based on the metabolic pathways in HEK293 MUL1(−/−) cells. Genome-wide RNA-sequencing data comparing HEK293 WT and MUL1(−/−) gene expression showed that 781 genes were downregulated whereas 822 genes were upregulated in MUL1(−/−) cells (FIG. 9A). The heatmap of the top 50 differentially expressed genes show excellent clustering and classification between both groups (FIG. 9B). Functional enrichment analysis of data identified that a large proportion of the regulated genes in HEK293 MUL1(−/−) cells are important for metabolic processes, including lipid, glucose, retinoic acid, carbohydrate and glutathione metabolism. This analysis indicated that deletion of MUL1 upregulates a group of genes including: CYP2E1, FABP6, PNPLA1, ACSBG1, AKR1C3, AKR1C1, and PIP5K1C that are involved in lipid transport, prostaglandin, progesterone, triglyceride and retinoic acid metabolic process. In addition, GPX4, PLCB2, PLPP4, PLPPR3, FADS1, FADS2, GSTP1 and GSTM2, genes involved in phospholipid and linoleic acid metabolic process were downregulated. Genes involved in glucose and pyruvate metabolism (IGF2, TNF, DCXR, ONECUT1, LDHC and PCK2) were also downregulated. A heatmap highlighting genes that are involved in the lipid, glucose, carbohydrate, glutathione, and retinoic acid metabolism, is shown in FIG. 9C. To take advantage of multi-omics datasets of HEK293 cells, a joint pathway analysis, employing the fold change values of the metabolites and genes in HEK293 MUL1(−/−) cells as compared to WT control cells, was conducted. The results from joint pathway analysis depicted in FIG. 9D indicate that glutathione metabolism, starch and sucrose metabolism, fatty acid (linoleic acid) metabolism, glycolysis and gluconeogenesis, glycine, serine, and threonine metabolism, glycolipid metabolism, and retinol metabolism were significantly interrupted in HEK293 MUL1(−/−) cells.

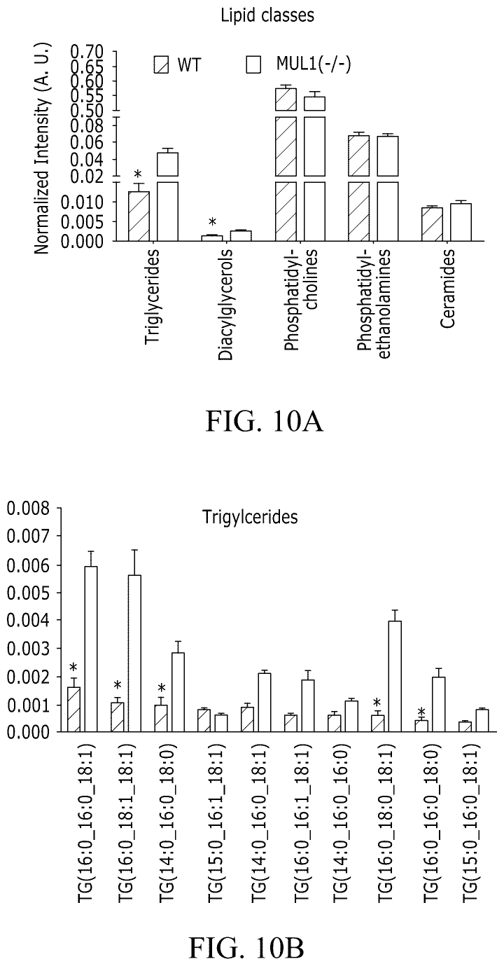

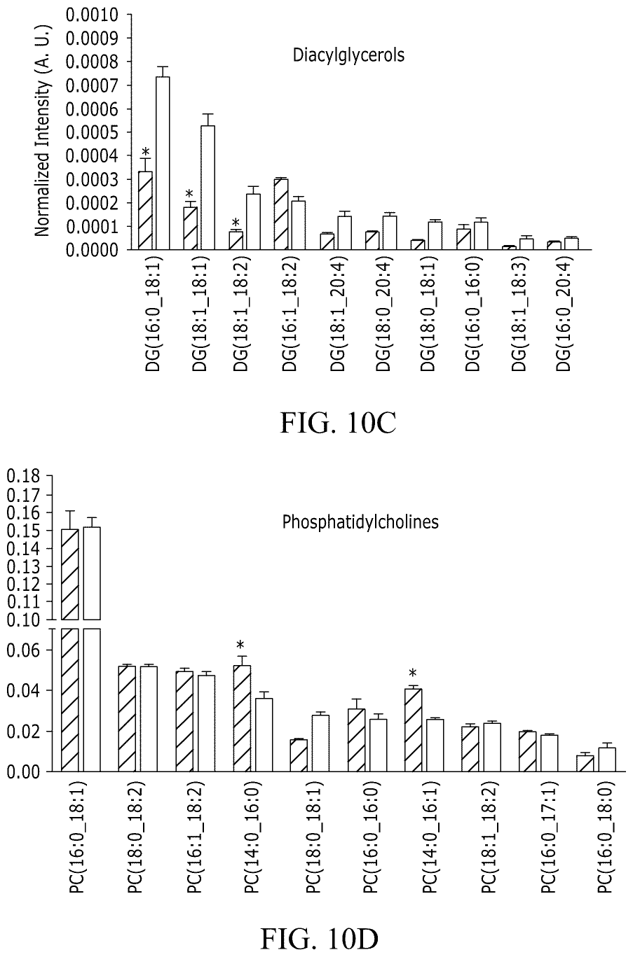

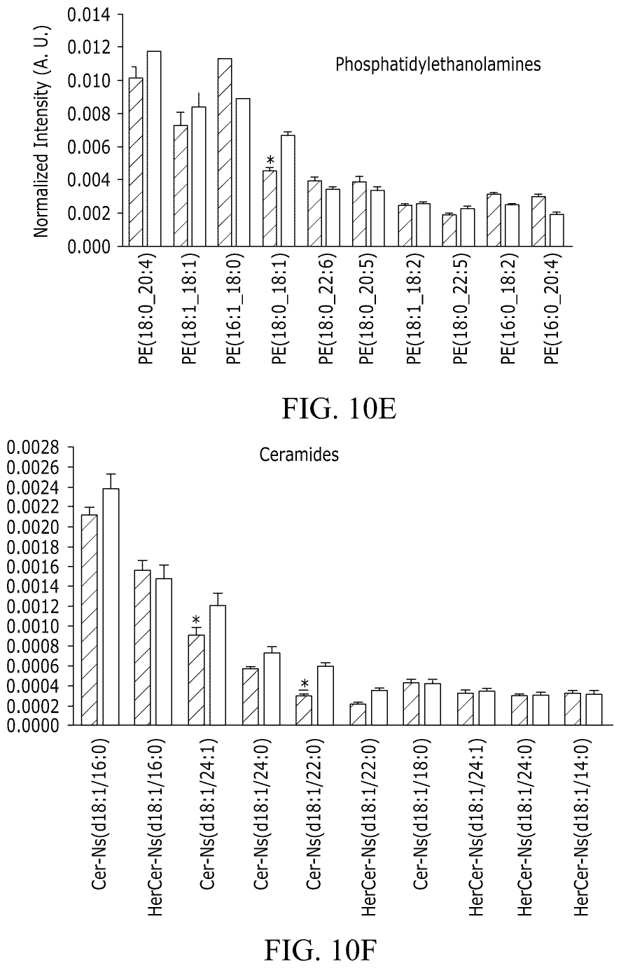

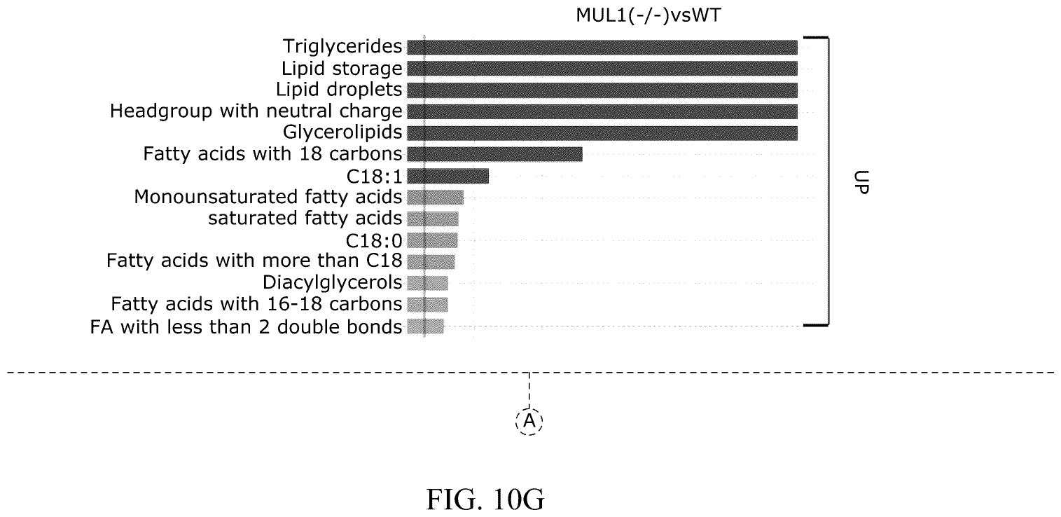

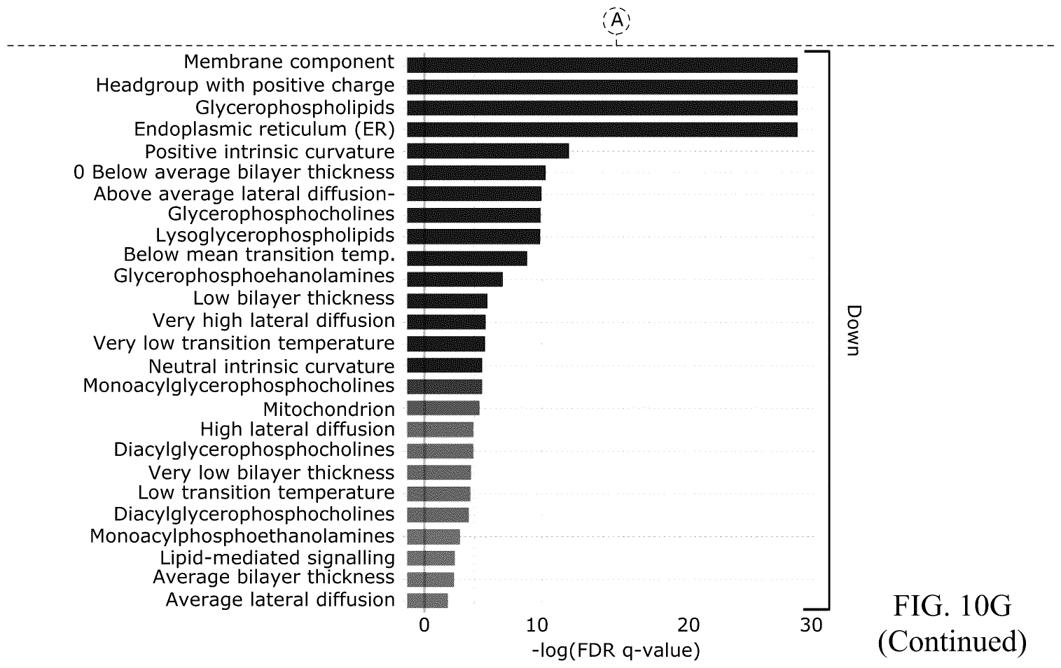

Perturbation in the lipid profile of HEK293 MUL1(−/−) cells. To monitor changes in the lipid profile LC-MS analysis was performed on HEK293 WT and MUL1(−/−) cells. The profiling of various lipid classes such as triglycerides (TGs), diacylglycerols (DGs), phosphatidylcholines (PCs), phosphatidylethanolamines (PEs), and ceramides strong changes in lipid metabolism enforced by the MUL1 knockout. The lipidomic profiling data demonstrated that the TGs and DGs are significantly higher in HEK293 MUL1(−/−) cells but there was no notable difference found for PCs, PEs, and ceramides (FIG. 10A). To provide more insight into lipid profiling of each lipid class, the 10 most abundant TGs, DGs, PCs, PEs, and ceramides were analyzed. TGs such as TG(16:0_16:0_18:1), TG(16:0_18:1_18:1), TG(14:0_16:0_18:0), TG(16:0_18:0_18:1), and TG(16:0_16:0_18:1) as well as DGs species i.e., DG(16:0_18:1), DG(18:1_18:1), and DG(18:1_18:2) were found significantly higher in MUL1(−/−) (FIGS. 10B-C). PC(14:0_16:0) and PC(14:0_16:1) and PE(18:0_20:4), and PE(18:0_18:1) were significantly lower in MUL1(−/−) cells whereas, PE(16:1_18:0) was found higher in WT cells (FIGS. 10D-E). Ceramide species such as CerNS(d18:1/24:1) and CerNS(d18:1/20:0) were significantly higher in MUL1(−/−) cells (FIG. 10F). Lipid Ontology (LION) enrichment analysis was carried out for annotation of lipids in untargeted lipidomic analysis. The results shown in FIG. 10G suggest that the most important components such as lipid storage, triglycerides, headgroup with a neutral charge, glycerolipids, fatty acids with 18 carbons, and C18:1 chain length were significantly upregulated in MUL1(−/−) cells. Lipids of membrane component, headgroup with a positive charge, glycerophospholipids, lipids of endoplasmic reticulum, lysoglycerophospholipids, glycerophosphocholines, and glycerophosphoethanolamines were significantly lower in HEK293 MUL1(−/−) cells.

### Discussion of Experimentation—Study One

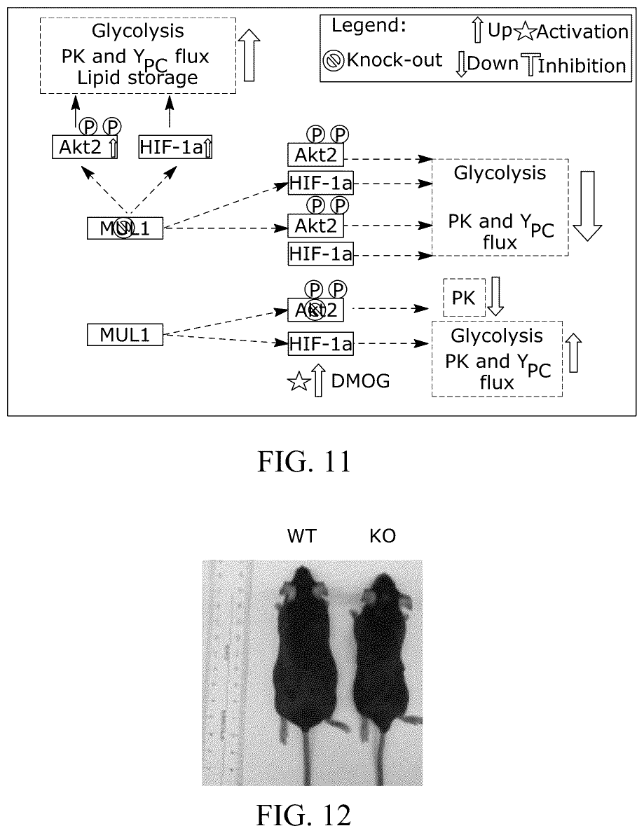

Metabolic reprogramming is the hallmark of cancer, but healthy mammalian cells can also modulate their metabolic state in response to fast growth demand or due to various conditions that adversely affect oxidative phosphorylation (OXPHOS), such as hypoxia (131; 133; 157). Metabolic regulation involves the coordinate function of numerous proteins located in different subcellular compartments including mitochondria, cytoplasm, and nucleus. Mitochondria continuously communicate their current bioenergetic and biosynthetic state to the rest of the cell through various signaling pathways and they can alter their function to accommodate changing metabolic demands (148). One of the many ways by which mitochondria communicate is through signaling proteins located in the outer mitochondrial membrane (OMM). As noted, the studies described herein are focused on one such protein, the mitochondrial MUL1 E3 ubiquitin ligase and its potential role in the regulation of metabolism. MUL1 is located in the OMM and its function has been implicated in mitophagy, cell death, mitochondrial dynamics, and innate immune response (167; 168; 37; 152; 26; 209; 22). As an E3 ubiquitin ligase, MUL1 interacts with various E2 ubiquitin conjugating enzymes to ubiquitinate specific substrates (60). MUL1 can perform two types of ubiquitination where either the lysine 48 (K48) or the lysine 63 (K63) of the ubiquitin is involved in the isopeptide linkage (136; 144). In addition, MUL1 can function as a SUMO E3 ligase to attach SUMO (small ubiquitin-like modifier) onto specific substrates (23; 25; 171). In the studies described herein, MUL1's K48-ubiquitination that invariably targets substrates for proteasomal degradation was investigated. At least four substrates are regulated and accumulate in MUL1(−/−) cells: ULK1, MFN2, HIF-1α, and Akt2 proteins (FIGS. 4A-E)) (28; 39; 132; 22). The studies described herein focused on Akt2 and HIF-1α coregulation by MUL1 since activation of these proteins is involved with metabolic phenotypes that favors glycolysis, a hallmark of cancer cells refers to as the Warburg effect (Lu et al., 2002; Pavlides et al., 2009). It was found that inactivation of MUL1 leads to suppression of OXPHOS and increased glycolysis. In addition, steady state flux rates show increased activity of Y_{PC} and PK flux in MUL1(−/−) cells. These metabolic changes can be reversed by specific Akt2 or HIF-1α inhibitors. Furthermore, data presented herein indicate that the metabolic phenotype of MUL1(−/−) cells is distinct from the glycolytic state observed in cancer cells (Warburg effect), where increased pyruvate dehydrogenase (PDH) and lactate dehydrogenase (LDH) switch OXPHOS to glycolysis (150; 84). Detailed metabolomics analyses of MUL1(−/−) cells, that clearly shows MUL1 has a very important role in both metabolic and lipidomic regulation, was performed. Detailed metabolomic and genomic analyses, using HEK293 WT and MUL1(−/−) cells, was employed to investigate the state of metabolic pathways. The results demonstrate there are significant differences in the metabolites and the gene expression profiles between these two cell lines. The multi-omics approach identified a number of metabolic pathways that were perturbed in the absence of MUL1. These include glutathione metabolism, starch and sucrose metabolism, fatty acid metabolism, glycolysis, glycine, serine, and threonine metabolism, glycolipid metabolism, and retinol metabolism. Lipidomic analysis showed significant accumulation of neutral head groups containing lipids such as triglycerides and diacylglycerides in MUL1(−/−) cells. The overall levels of other lipid species such as phosphatidylcholines, phosphatidylethanolamines, and ceramides were unaffected by MUL1 inactivation in HEK293 cells. FIG. 11 summarizes the proposed MUL1's function and pathway in the regulation of metabolism based on the data presented here.

The multifaceted analysis shows that MUL1 is involved in the regulation of metabolism and its inactivation/downregulation can lead to a distinct metabolic state not previously described. MUL1 protein level is known to be regulated but the mechanism(s) is not fully characterized. Previous studies of the instant inventor's identified Omi/HtrA2, a serine protease located in the mitochondrial intermembrane space (IMS), as a major regulator of MUL1 protein level (59). Omi/HtrA2 has a role in protein quality control within the IMS, and its activity is modulated by oxidative stress, the HAX1 protein, as well as by the NDUFA13 subunit of mitochondrial respiratory chain complex I (175; 77; 102; 100; 103; 101). In addition, K48-autoubiquitination of MUL1 is another potential mechanism that can target the ligase for degradation and regulate its protein level (39; 199).

Besides the involvement of Akt2 and HIF-1α in the regulation of metabolism by MUL1, the participation of other MUL1-substrate proteins, such as the MFN2 and ULK1 proteins, cannot be excluded. Besides Akt2 and HIF-1α, accumulation of MFN2 and ULK1 is also observed in MUL1(−/−) cells. In addition, MFN2 has been implicated in the regulation of metabolism in cancer cells and shown to interacts with PKM2 (149; 84). ULK1 plays a major role in autophagy but can also phosphorylate key glycolytic enzymes, such as hexokinase (HK), phosphofructokinase 1 (PFK1), enolase 1 (ENO1), and fructose-1,6-bisphosphatase (FBP1), in response to nutritional deprivation (79). This dual parallel function of ULK1 can sustain the activity of multiple glycolytic enzymes and support metabolic homeostasis during amino acid and growth factor deprivation (79). In the studies described herein, specific inhibitors were used to establish that Akt2 and HIF-1α are the main “drivers” of the MUL1(−/−) metabolic phenotype; any potential role MFN2 and/or ULK1 might have in this process will be downstream of these two proteins.

### Introduction to Experimentation—Study Two—In Vivo Aspects

A new function for the mitochondrial MUL1 E3 ubiquitin ligase in the regulation of metabolism was identified. The mechanism involves the K48-polyubiquitinating function of the ligase and the coregulation of Akt2 and HIF-1α proteins. The accumulation and coactivation of Akt2 and HIF-1α in MUL1(−/−) cells drives and maintains this new metabolic phenotype characterized by activated pyruvate carboxylation and PK flux, along with increased aerobic glycolysis. In addition, MUL1(−/−) cells have a distinct lipid metabolism characterized by increased triglyceride and diacylglycerol storage. These results support a very important role for MUL1 ligase in the regulation of mitochondrial metabolism and lipogenesis including a new metabolic state of aerobic glycolysis.

### Experimentation Using MUL1 (−/−) Knockout (KO) Mice

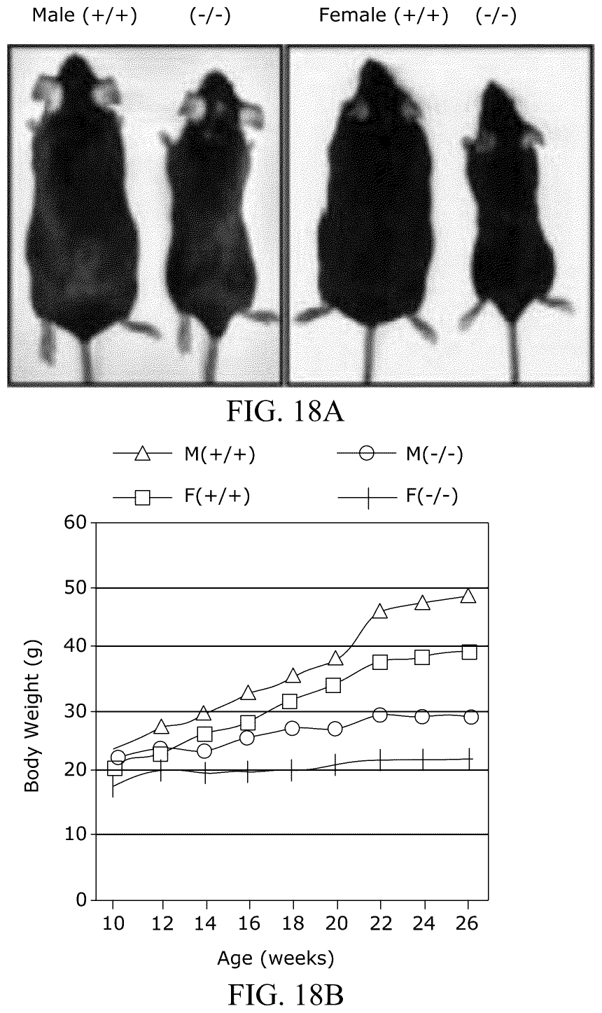

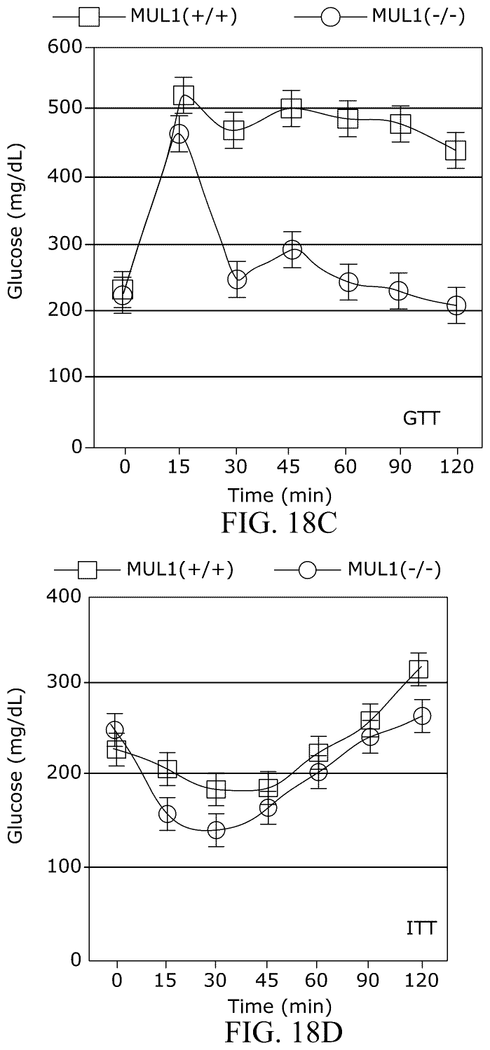

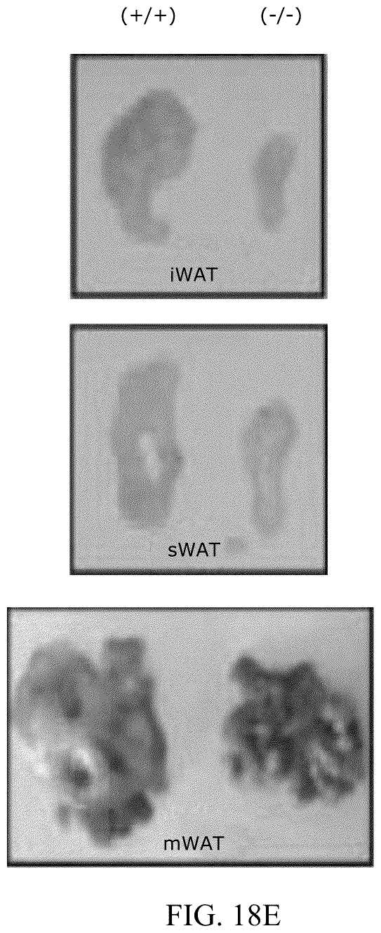

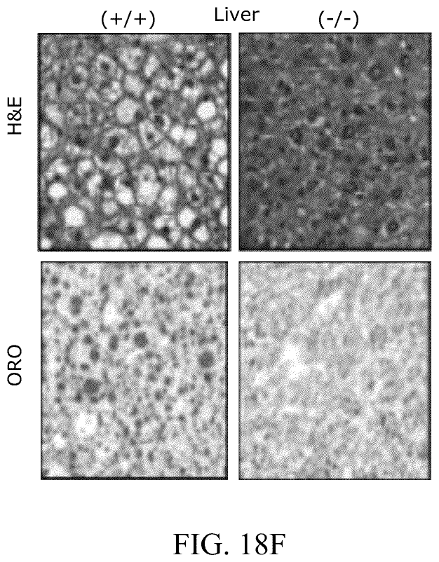

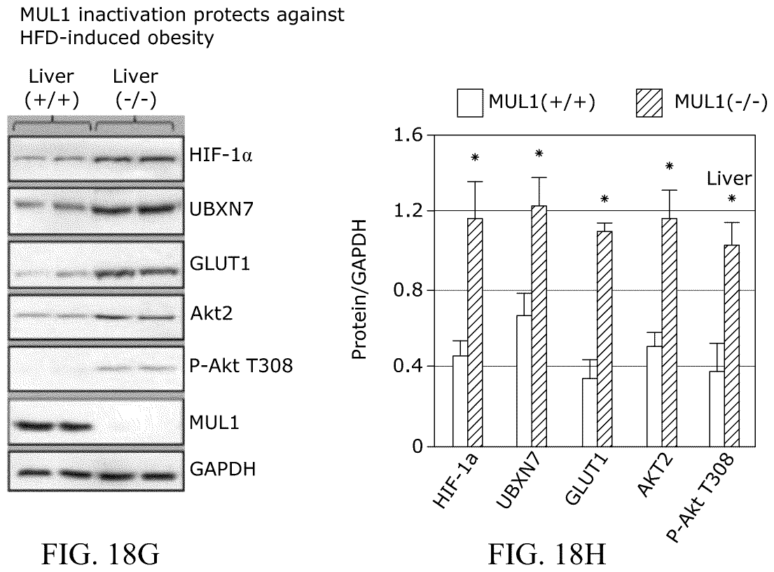

In the experimentation previously discussed herein, cultured cells were used as a convenient system to identify and dissect the various components involved in the MUL1-mediated pathway of metabolic regulation. The next step in this study is development of insight into the targets of MUL1 in vivo and to assess the downstream effects on metabolism. Mice that carry a global inactivation of MUL1 will be used in this next step. These animals have a metabolic phenotype, and the preliminary data show they are resistant to HFD-induced obesity (and associated metabolic systems), fatty liver, insulin resistance, and glucose intolerance (FIGS. 18A-H). These mice also have a very high rate of oxidative phosphorylation and lipid metabolism. The metabolic phenotype of the MUL1(−/−) has not been previously investigated and supports the concept that mitochondrial MUL1 is a major regulator of cell metabolism.

It was investigated if the data using HEK293 MUL1(−/−) cells is consistent with the phenotype of Mul1(−/−) mice that carry a global inactivation of MUL1. High fat diet (HFD) is commonly used as a modulator of rodent metabolism that models to some extent the western diet and persistent overnutrition. The HFD mouse is characterized by increasing obesity, impaired glucose tolerance, fatty acid deposition in the liver, and increased liver inflammation, among other phenotypical changes (108). Mul1(−/−) and WT mice were placed on a high fat diet (HFD 60% fat, 20% protein, 20% carbohydrates) for 16 weeks to induce obesity.

### A) Analysis of Energy Metabolism in Mul1(−/−) Mice on a HFD Using Metabolic Cages

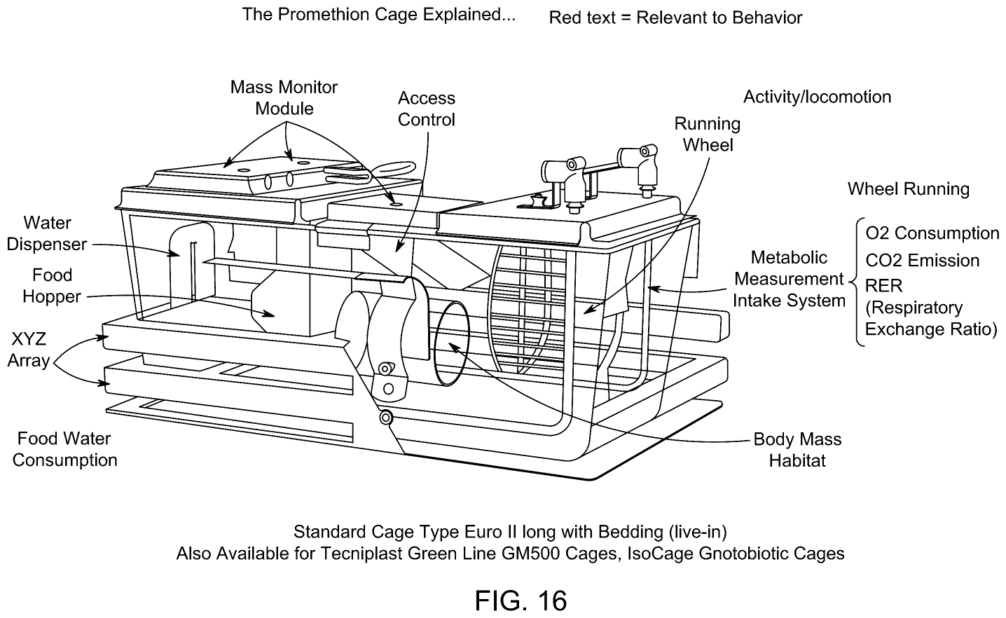

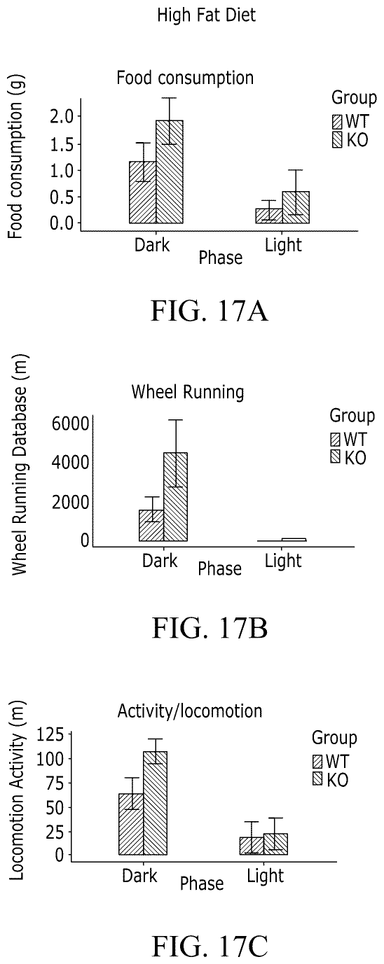

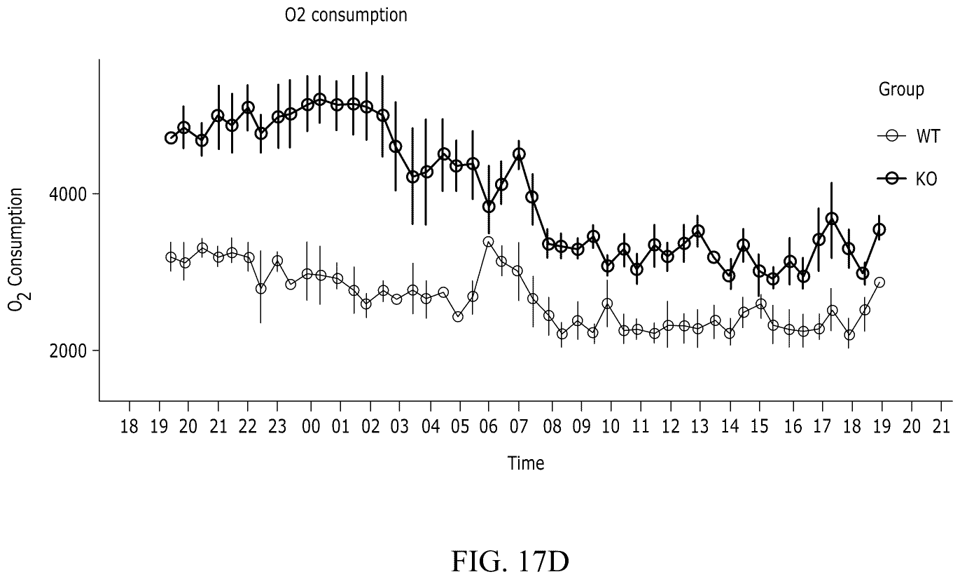

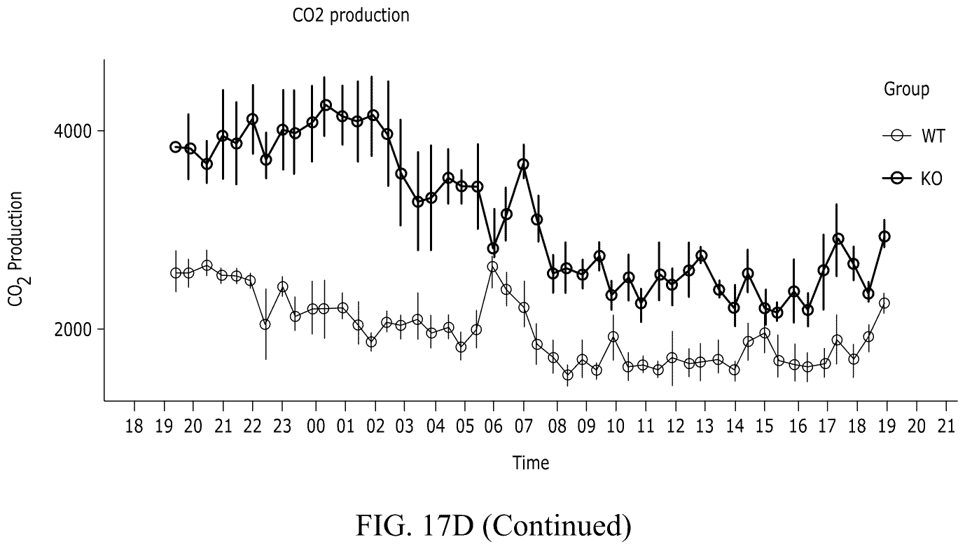

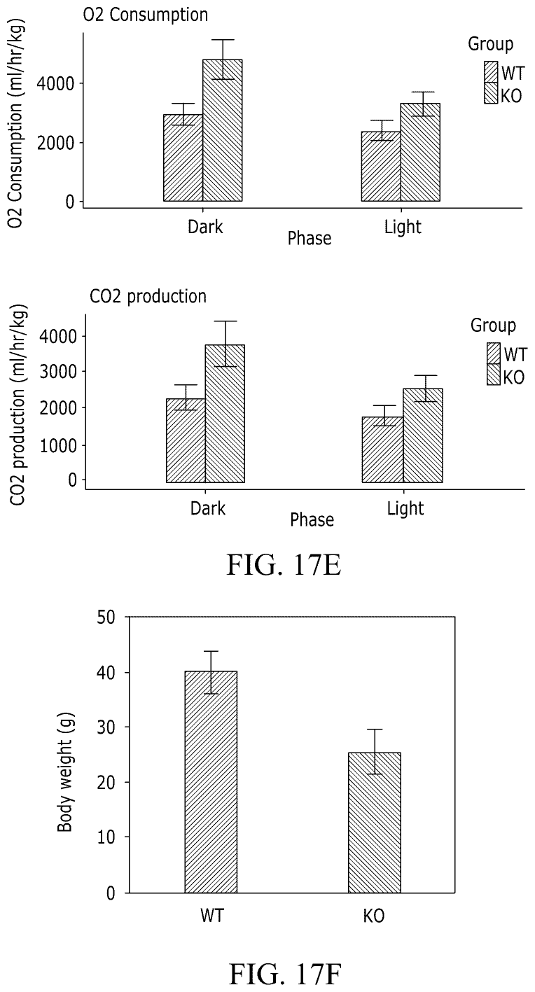

After 8 weeks on HFD animals were placed individually in a 16-channel Promethion system gas-tight metabolic cage (FIG. 16) kept at 22° C. (Sable System International). Mice were acclimated for 24 hours before the energy metabolism and the various parameters were recorded. The parameters monitored included water, food consumption, total animal activity, the volume of Oxygen consumption (VO₂), CO₂ release (VCO₂) and the respiratory exchange ratio (68). Results shown are based on animal weight and calculated as described (69, 70). FIGS. 17A-D show representative data obtained from Mul1(−/−) and WT mice, with both groups of animals being kept on HFD. Mul1(−/−) mice have a higher VO₂ and VCO₂ indicating an increased metabolic rate and oxidation of fatty acids compared to WT animals (FIGS. 17D-E). In addition, the activity is significantly higher in the HFD Mul1(−/−) mice (FIGS. 17B-C), and the food intake is not significantly different between the two groups (FIG. 17A)(71).

B) Mul1(−/−) Mice have a Metabolic Phenotype, and they are Resistant to HFD(High-Fat Diet)-Induced Obesity and Metabolic Syndrome.

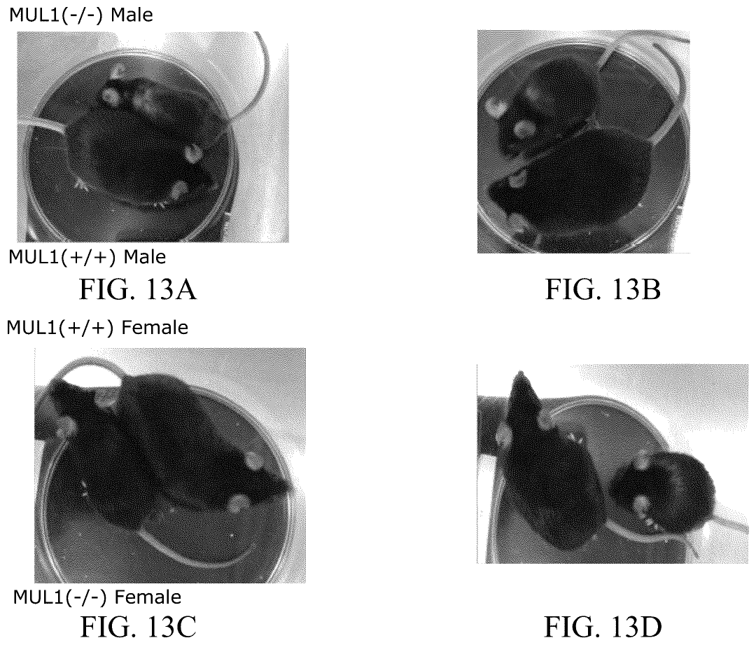

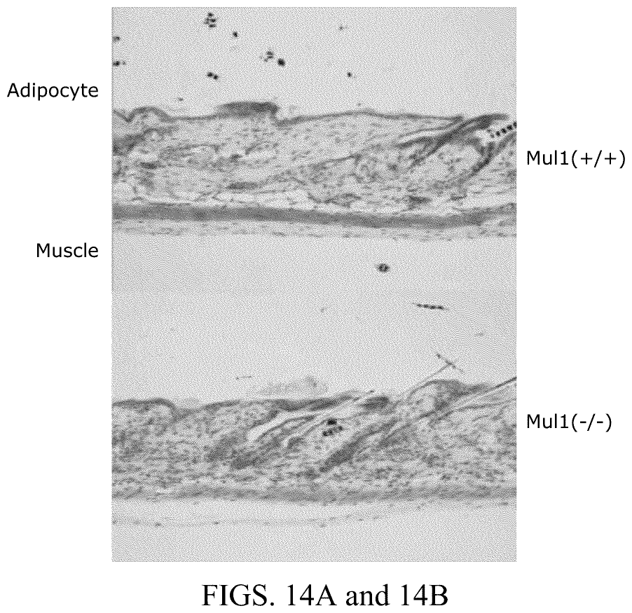

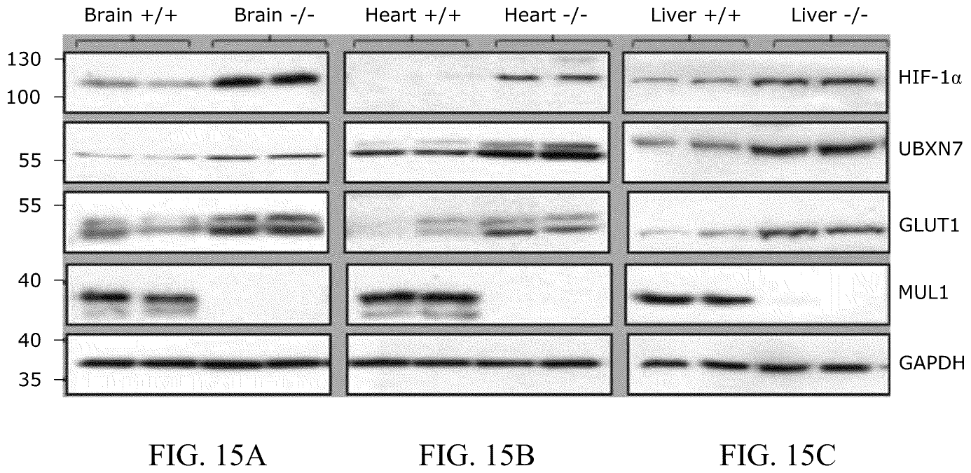

At 16 weeks on the HFD, Mul1(+/+) animals became obese, their body weight more than doubled (FIG. 12, FIGS. 13A-D, and FIG. 18A). In contrast, Mul1(−/−) animals were resistant to HFD-induced obesity with no significant change in their body weight (FIG. 18B). Insulin and glucose tolerance assays were performed and, in both cases, the Mul1(−/−) mice performed significantly better than the Mul1(+/+) animal (FIGS. 18C-D). Consistent with the effect on weight accumulation, subcutaneous, epididymal, and mesenteric adipose tissue mass was unaffected in Mul1(−/−) mice on HFD (FIG. 18E). Histological analysis of the liver clearly demonstrates that MUL1(+/+) but not Mul1(−/−) mice developed severe steatosis at 16 weeks on HFD. Staining with Oil Red O (ORO) further confirmed a massive lipid deposition in the liver of MUL1(+/+) control mice, which is absent in the liver of Mul1(−/−) animals (FIG. 18F). Tissue extracts from livers of Mul1(−/−) and WT mice were analyzed by Western blotting using specific antibodies (FIG. 18G). The bar graph of FIG. 17H shows densitometric analysis of protein expression from FIG. 18G normalized against GAPDH.

### Inactivation of Mitochondrial MUL1 E3 Ubiquitin Ligase Inhibits Lipogenesis

### Introduction

The primary function of mitochondrial metabolism is to provide ATP, through oxidative phosphorylation (OXPHOS), to support the energy demand of the cells [163,164]. In addition, mitochondria play a major role in the biosynthesis of precursors used for macromolecules such as lipids, proteins, DNA, and RNA. These bioenergetic and biosynthetic functions of mitochondria are regulated and adjusted in response to changes in the physiological environment [165]. The regulation of mitochondrial metabolism involves numerous proteins with diverse functions as well as effective communication between mitochondria with the rest of the cell. MUL1 (also known as Mulan, MAPL, GIDE, and HADES) is one of three mitochondrial E3 ubiquitin ligases, the other two being MARCH, and RNF185 [23,166,24,30,167]. MUL1 is located on the outer mitochondrial membrane (OMM) and its function has been implicated in the regulation of mitophagy, cell death, mitochondrial dynamics, innate immune response, and very recently mitochondrial metabolism [168,25, 26,169,170,59]. MUL1 can perform K48- and K63-ubiquitination, as well as SUMOylation targeting specific substrates located in the OMM or in the vicinity of mitochondria [22,25,26,171-173].

In Study One, using human cell lines, a unique function of MUL1 in the regulation of mitochondrial metabolic homeostasis [22,34,59,60,174,175] was uncovered. In Study Two, the phenotype of mice carrying a whole-body inactivation of the Mul1 gene was investigated. Mul1(−/−) animals, fed a standard diet (ND), are slightly smaller and leaner than, wildtype animals, but developmentally normal and exhibit features of impaired metabolomic and lipidomic function. Using metabolic cages, the whole-body energy expenditure and metabolism of Mul1(−/−) mice was monitored and shown to be different than their Mul1(+/+) littermates. In addition, when the Mul1(−/−) mice were placed on a high fat diet (HFD) they exhibited increased oxygen consumption (higher metabolic rate), robust resistance to HFD-induced obesity, and intact thermoregulation upon cold challenge. Metabolomic and lipidomic analyses as well as Genome-wide mRNA sequencing of the liver of Mul1(−/−) mice on HFD, identified several metabolic enzymes involved in lipogenesis and b-oxidation that are differentially regulated in the absence of MUL1. One of these enzymes, markedly downregulated in the liver of Mul1(−/−) mice on HFD, is the Stearoyl-CoA Desaturase 1 (SCD1) protein. SCD1 is a widely studied enzyme involved in the biosynthesis of monounsaturated fatty acids. Animals with a targeted disruption of Scd1 gene have a phenotype that closely resembles the corresponding phenotype of Mul1(−/−) mice [177-181]. This includes resistance to HFD, reduced fatty acid synthesis, and increased energy expenditure. In addition, several enzymes, that have previously been shown to be dysregulated in the absence of SCD1, such as phosphorylated AMP-activated protein kinase (pAMPK), acetyl-CoA carboxylase 1 (ACC1), fatty acid synthase (FASN) and carnitine palmitoyltransferase I (CPT1), were also affected in a similar manner in the liver of Mul1(−/−) animals on HFD. These results suggest that mitochondrial MUL1 ligase is an important regulator of lipogenesis and fatty acid oxidation especially during HFD conditions. Mul1 inactivation downregulates the expression of Scd1 gene and inhibits SCD1 protein induction, that normally occurs by HFD, in the liver and white adipose tissue (WAT). Consequently, Mul1(−/−) mice are characterized by high metabolic rate, thinness, and resistance to HFD-induced obesity and associated metabolic syndrome. This data supports a new role for MUL1 ligase as an important metabolic regulator of lipogenesis and β-oxidation. It is proposed that this new function of MUL1 is mediated primarily through the regulation of SCD1 and other key enzymes involved in lipid metabolism and β-oxidation. Furthermore, these studies advocate that MUL1 ubiquitin ligase is a promising target in the development of therapeutic interventions for obesity and associated metabolic disease.

### Material and Methods

Using metabolic cages, whole body energy expenditure, metabolism, and thermoregulation of the Mul1(−/−) mice under standard diet (ND) or high fat diet (HFD) was monitored. The effect of Mul1 inactivation on body weight, HFD-induced adiposity, fatty liver, glucose intolerance, and insulin resistance was examined. Global metabolomics, lipidomic, and genome-wide mRNA sequencing using liver from Mul1(+/+) and Mul1(−/−) animals on HFD was performed. The expression level of key proteins involved in lipogenesis and their regulation in the absence of MUL1 was monitored by SDS-PAGE and Western blot analysis.

### Experimental Animals

Male and female wild-type, Mul1(+/+) mice, as well as whole-body Mutt deficient, Mul1(−/−) mice were used throughout these studies. Mul1(−/−) mice have been described (22), and were generously provided by Dr. Heidi Mcbride, McGill University, Montreal, Quebec Canada. Mul1(−/−) mice were obtained by breeding heterozygous Mul1(+/−) animals. All animals were maintained at 22°±1° C. on a 12-hour/12-hour light/dark cycle and had free access to food and water. Mice at 8 weeks of age (n=6) were maintained for 16 weeks on a ND where 18% of the calories are derived from fat, or on a HFD where 60% of calories are obtained from fat (Research Diets D12492). After this time, the animals were euthanized, liver and fat tissues were collected and used for histology, LC-MS metabolomics, global lipidomic, mRNA sequencing and Western blot analysis. All experimental protocols were conducted in compliance with animal procedures and approved by the University of Central Florida Institutional Animal Care and Use Committee (IACUC).

### Glucose and Insulin Tolerance Tests

Glucose tolerance test (GTT), and insulin tolerance tests (ITT) were performed on Mul1(+/+) and Mul1(−/−) mice that were established on HFD for 16 weeks or maintained on ND for the same time. Animals were fasted for 16 hours (GTT) or for 6 hours (ITT). For GTT test, animals received 1 g/kg of body weight glucose administered intraperitoneally (i.p.). After injection, blood glucose was monitored using a glucose meter on tail bleeds at various time points up to 2 hours. For ITT, animals were injected i.p. with 1 IU/kg body weight insulin (Novolin R U-100 Thermo Fisher Scientific) followed by blood glucose measurements every 15 minutes for up to 2 hours.

### Liver Histology

Liver tissue was obtained from Mul1(+/+) and Mul1(−/−) mice following a HFD for 16 weeks and stained with H&E or Oil Red O as described [182]. Briefly, liver tissues from mice were fixed overnight in 10% neutral formalin and embedded in paraffin. Paraffin-embedded tissues were cut into sections and stained with hematoxylin and eosin (H&E) for assessment of liver histology. For the Oil Red O staining, freshly frozen liver tissues were embedded in Tissue-Tek OCT in a cryostat mold. The sections were then stained with Oil Red O (ORO) to monitor lipid accumulation [183].

### Indirect Calorimetry

The indirect calorimetry cage system (Promethion Sable Systems International) was used to measure oxygen consumption (VO2), CO2 emission (VCO2), energy expenditure (EE), food intake, water consumption, locomotor activity, and animal weight. The software captures metabolic parameters in each cage even 5 min. Mice were weighed and then individually housed in cages for 6 days. Animals were first acclimated to the cages for 1 day before experimental data were collected for 96 hours. Mice were maintained on a 12-hr/12-hr light/dark cycle and had free access to food and water for the duration of the experiment. Data were analyzed using CalR, a web-based analysis tool of indirect calorimetry to measure physiological energy balance [69,184].

### Thermoregulation

Four-month-old Mul1(+/+) and Mul1(−/−) male mice (n=5) were subcutaneously implanted with a temperature transponder (IMI-400 BMDS Avidity Science) two weeks before the beginning of indirect calorimetric experiments. The transponder is a miniature glass-encapsulated microchip that can be scanned by an appropriate reader device. Animals were maintained on a ND, housed at 22° C., with free access to food and water throughout the experiment. Mice were weighted and transferred to individual metabolic cages for 48 hours at 22° C. followed by cold challenge at 4° C. for 48 hours. The temperature transition from 22° C. to 4° C. was carried out over 48 hours. Body temperature, O2 consumption, CO2 emission, food and water intake, energy expenditure, and activity were monitored using a calorimetry cage system (Promethion Sable Systems International). Data were analyzed and plotted using Ca IR, web-based analysis tool of indirect calorimetry to measure physiological energy balance [184-186].

### SDS-PAGE and Western Blot Analysis

Mouse liver or subcutaneous white adipose (sWAT) tissues were homogenized using a Triton X-100 based lysis buffer (1% Triton X-100, 10% glycerol, 150 mM NaCl, 20 mM Tris pH 7.5, 2 mM EDTA) in the presence of protease inhibitors tablets (Thermo Fisher Scientific). Approximately 40 μg of whole tissue extract was resuspended in SDS sample buffer, boiled for 5 minutes and the proteins resolved by SDS-PAGE. They were then transferred onto PVDF Immobilon membranes (Millipore) using a semi-dry cell transfer blot (Bio-Rad) and placed in 4% nonfat dry milk in TBST buffer (25 mM Tris-HCl pH 8.0, 125 mM NaCl, 0.1% Tween 20) to block nonspecific binding of the membrane. The membranes were incubated with the indicated primary antibodies: MUL1, rabbit polyclonal antibodies (SIGMA), UCP1 (home grown Periasamy's Lab) SCD1, CPT1(ProteinTech), AMPK, pAMPK, ACC1, pACC1 (Cell Signaling Technology), FASN (ABclonal) β-actin, GAPDH and HSP90 (Santa Cruz Biotechnology). Secondary peroxidase-conjugated goat anti-rabbit or goat anti-mouse antibodies (Jackson ImmunoResearch) were used at 1:10,000 dilution; the membrane was then visualized by enhanced chemiluminescence (ECL) (Thermo Fisher Scientific).

### LC-MS Analysis of Mouse Liver

Liver tissue from Mul1(+/+) and Mul1(−/−) mice on HFD were subjected to the Folch extraction to separate lipids and polar metabolites. The polar metabolites were analyzed on Thermo QExactive Orbitrap mass spectrometer equipped with UHPLC. Samples were analyzed in positive and negative modes using heated electrospray ionization (HESI) source. Data were analyzed with MZmine and features were aligned for identification across samples. The metabolites were searched against the Southeastern Center for Integrated Metabolomics (SECIM) metabolite library using retention time and corresponding mass spectral data. Lipids isolated from Folch extraction procedure were also analyzed by the same instrument. Lipidomics data were analyzed using the LipidMatch software and identified lipid entities were exported in a tabular form [174,187].

### Metabolomics Data Analysis

The intensity of metabolites was normalized by the weight of the liver tissue used in the extraction of metabolites. MetaboAnalyst software was utilized for metabolomics and joint metabolic pathway analysis of genes and metabolites. Fold-change normalized LC-MS data was imported into the MetaboAnalyst software for statistical analysis. Joint metabolic pathway analysis was performed with uniport protein ID and HMDB ID of gene and metabolites, respectively. The fold change of genes and metabolites between the Mul1(−/−) and Mul1(+/+) HFD-liver was used to perform joint metabolic pathway analysis. The metabolomics panel of significantly different metabolites between Mul1(+/+) HFD-liver and Mul1(−/−) HFD-liver was identified via t-test in MetaboAnalyst [188].

### Lipidomic Data Analysis

The profiling of lipids was carried out using the normalized individual lipid to the liver tissue weight utilized for each sample. The top 10 most abundant lipids in each class are displayed and expressed as mean±SEM (n=4). Enrichment analysis between Mul1(+/+) HFD-liver and Mul1(−/−) HFD-liver in the ranking mode by lipid ontology (LION) interface was used to search for significantly different lipids [189]. The cut-off value of significantly up- or down-regulated lipids in Mul1(−/−) HFD-liver with respect to Mul1(+/+) HFD-liver (p<0.05) was determined and the data represented as −log FDR q-values.

### RNA Sequencing Analysis

Standard RNA-sequencing for gene profiling expression of protein-coding sequences (mRNA) was performed using liver tissues from Mul1(+/+) and Mul1(−/−) mice on HFD. Approximately 100 mg of tissue obtained from Mul1(+/+) and Mul1(−/−) animals (n=3 per group) was used for library construction, DNA sequencing, and data analysis (GENEWIZ Global). Using DESeq2, a comparison of gene expression between the defined groups of samples was performed. The Wald test was used to generate p-values and log 2 fold changes. Genes with an adjusted P value <0.05 (P<0.05) and absolute log 2 fold change >1 were labelled as differentially expressed genes. Significantly differentially expressed genes were clustered by their gene ontology and the enrichment of gene ontology terms was tested using Fisher exact test. Heatmaps of the top fifty, as well as genes specifically involved in fatty acid metabolism, lipid and phospholipid biosynthesis metabolomic processes, were analyzed using RStudio, the identified differentially expressed genes were used to generate a heatmap using Heatmap.2 software [190].

### Statistical Analysis

All quantitative data are expressed as mean±S. D. or ±SEM of three or four independent experiments. Following Western blot analysis, the optical densities of blot bands were determined using ImageJ software. Protein/β-actin ratios were obtained based on the densitometry data, and the differences among groups were analyzed by one tailed Student's t test. A value of p<0.05 was considered significant. For metabolomic data analysis (LC-MS metabolomic, lipidomic) as well as RNA-seq a value of P<0.05 was considered significant.

### Results—Study Two

It was found that Mul1(−/−) animals have a metabolic phenotype that confers robust resistance to HFD-induced obesity. Several metabolic and lipidomic pathways are perturbed in the liver of Mul1(−/−) animals on HFD, particularly the one driven by Stearoyl-CoA Desaturase 1 (SCD1), a key regulator of lipid metabolism and obesity. In addition, key enzymes involved in lipogenesis and fatty acid oxidation such as ACC1, FASN, AMPK, and CTP1 were also modulated. The concerted deregulation of these enzymes, in the absence of MUL1, causes reduced fat storage and increased fatty acid oxidation.

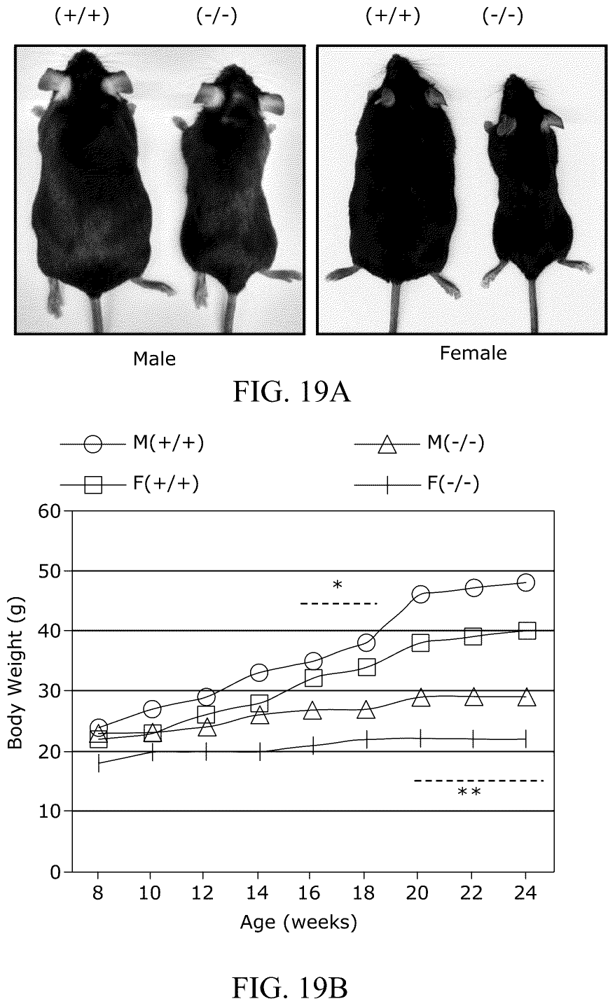

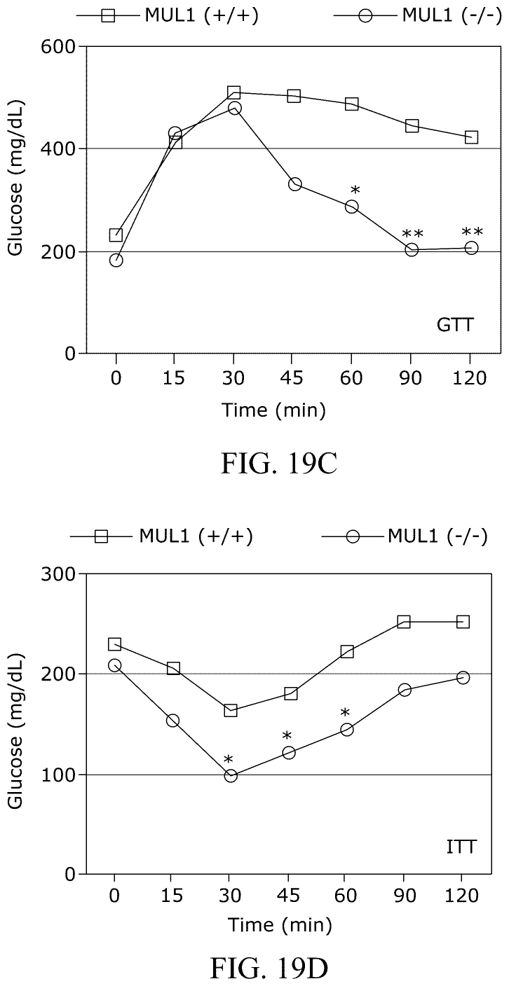

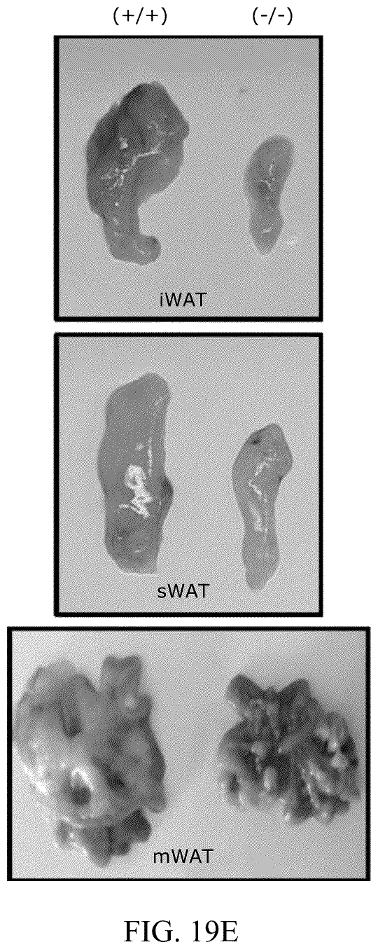

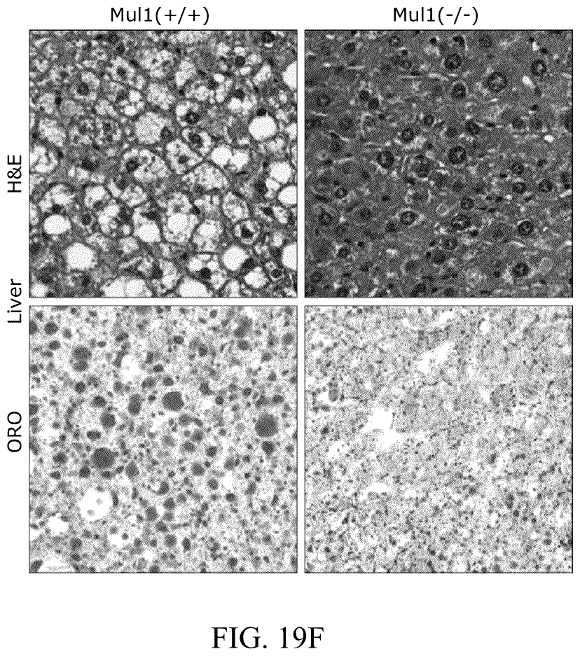

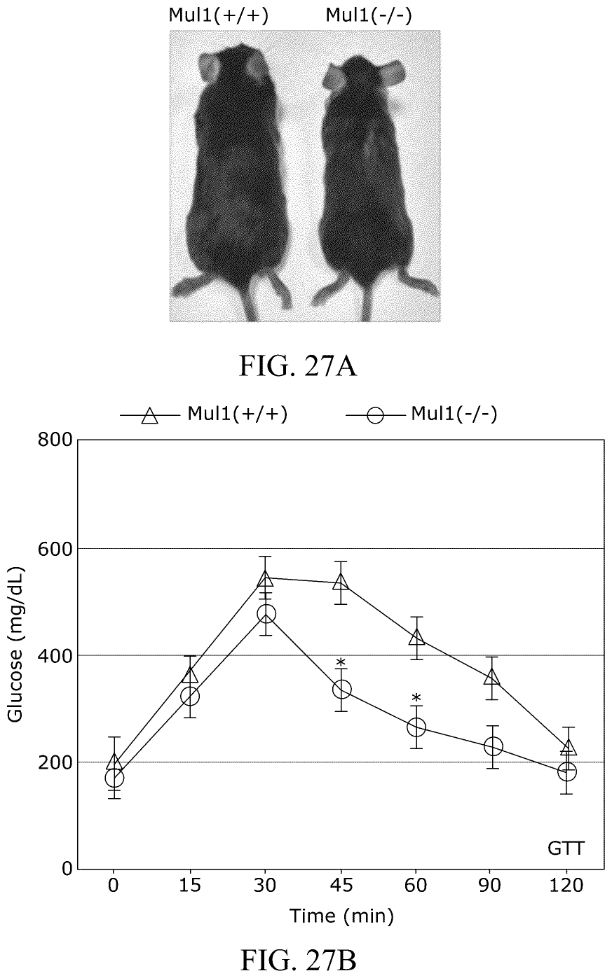

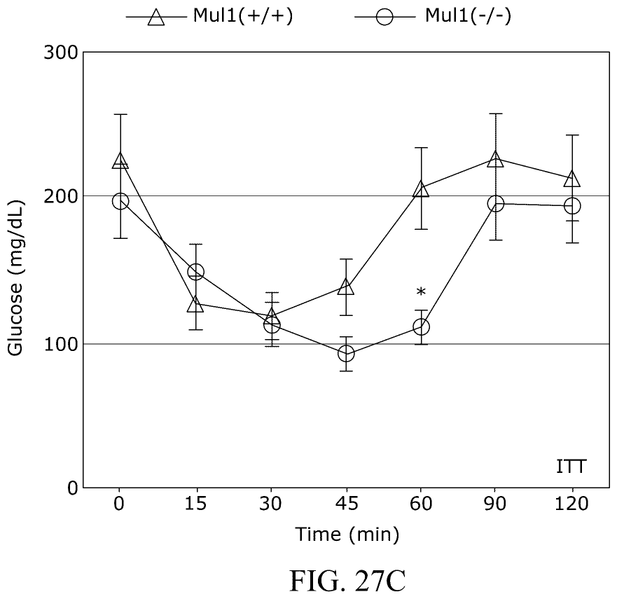

Genetic ablation of Mul1 in mice confers resistance to HFD-induced obesity—These studies, using human cell lines HEK293 and HeLa, have identified a new role for MUL1 ligase in the regulation of mitochondrial metabolism [174]. In the present study whether whole-body inactivation of Mul1 can affect the metabolism of the animals was investigated. Mul1(−/−) mice fed a ND are slightly smaller, leaner, and have improved glucose tolerance and better insulin sensitivity than their Mul1(+/+) littermates (FIG. 27A-C). The state of lipogenesis and adiposity of Mul1(−/−) mice was monitored by placing them on a HFD for 16 weeks. After this time Mul1(+/+) animals exhibited all the symptoms of HFD-induced obesity including a significant increase in body weight (FIGS. 19A-B). On the contrary, Mul1(−/−) animals showed resistance to HFD-induced obesity with very little increase in body weight (FIG. 19B). This effect was manifested in both male and female animals (FIG. 19A-B). Glucose tolerance and insulin resistance was also monitored in both Mul1(−/−) and Mul1(+/+) animals on HFD. FIGS. 19C-D show that Mul1(+/+) animals exhibited glucose intolerance, after a glucose load, that is typically associated with obesity and reduced sensitivity to insulin. Mul1(−/−) animals had improved glucose tolerance and better insulin sensitivity compared to their Mul1(+/+) littermates. In addition, Mul1(−/−) mice displayed a marked reduction in adiposity throughout iWAT, sWAT, and mWAT shown in the representative images in FIG. 19E. Since hepatic steatosis is a key feature of obesity, liver samples obtained from Mul1(+/+) and Mul1(−/−) animals and stained with H&E were compared. FIG. 19F (top panel) shows Mul1(+/+) mice exhibit clear signs of steatosis characterized by hepatocyte ballooning and accumulation of vacuoles. This steatosis phenotype was absent in Mul1(−/−) animals. In addition, Oil Red O staining of liver tissue shows that Mul1(−/−) mice have marked reduction in lipid accumulation compared to Mul1(+/+) animals (FIG. 19F, lower panel).

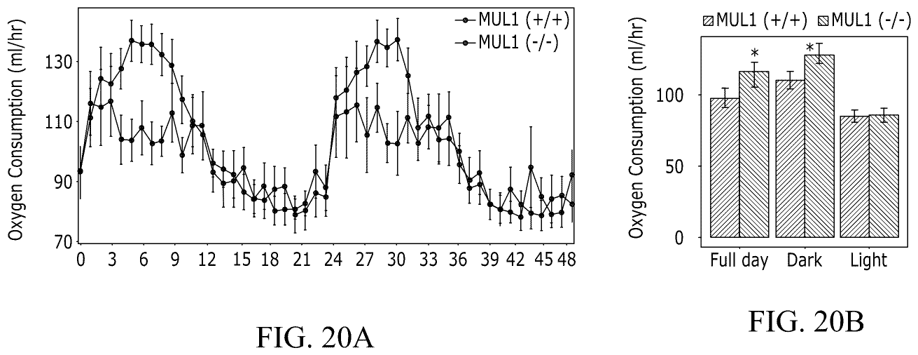

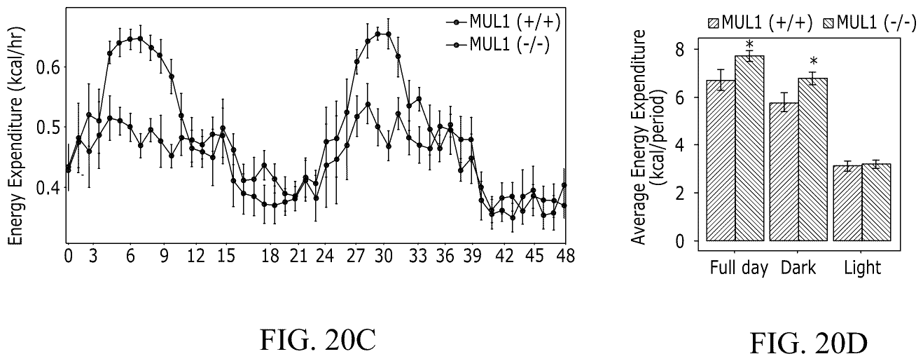

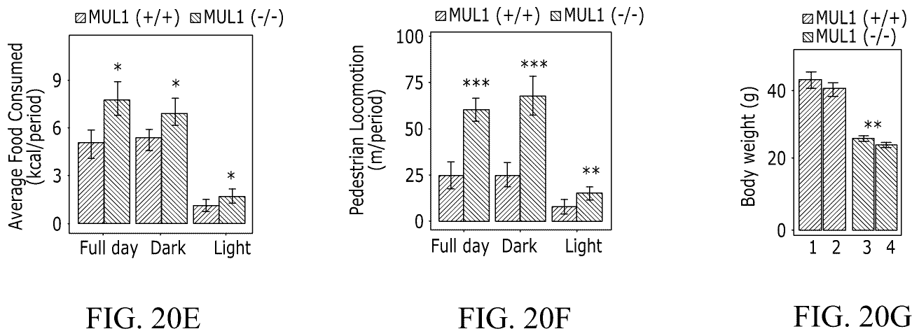

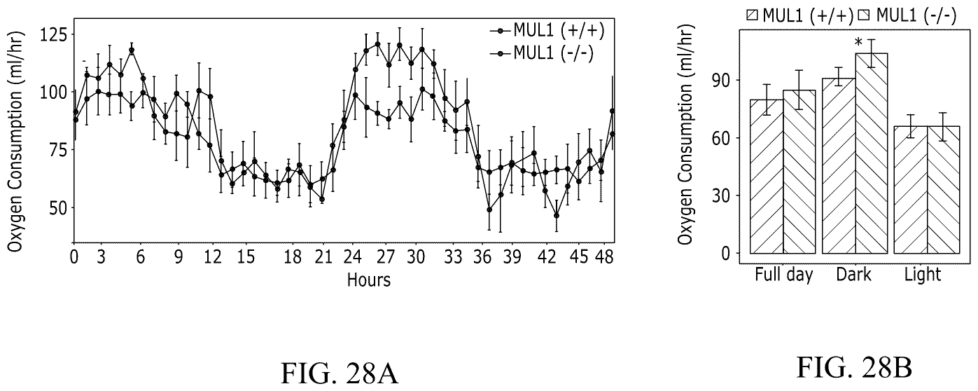

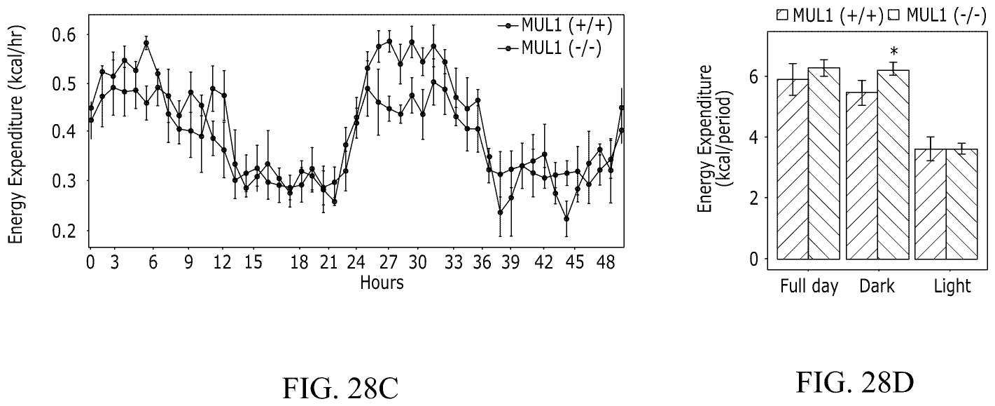

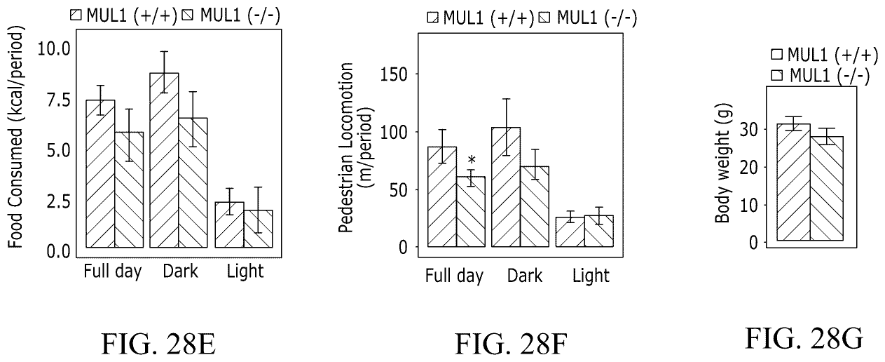

Mul1(−/−) mice on HFD have increased oxygen consumption and energy expenditure—To investigate whether the resistance of the Mul1(−/−) mice to HFD-induced obesity was caused by changes in energy expenditure and metabolism, Mul1(−/−) and Mul1(+/+) mice were placed on ND or HFD and a Promethion metabolic cage system. Mul1(−/−) mice on ND exhibited an increased rate of oxygen consumption (VO2) as well as energy expenditure compared to Mul1(+/+) animals (FIGS. 28A-D). This difference in oxygen consumption was even more pronounced when animals were maintained on the HFD (FIGS. 20A-B). There was also increased energy expenditure of Mul1(−/−) animals during the feeding phase (dark cycle) indicating their preference for lipids as an energy source over carbohydrates (FIGS. 20C-D). Additionally, Mul1(−/−) mice had significantly higher food consumption (FIG. 20E), and high activity locomotion (FIG. 20F). FIG. 20G shows the body weight of the animals at the start (bars 1 and 3) or the end (bars 2 and 4) of the experiment (n=5).

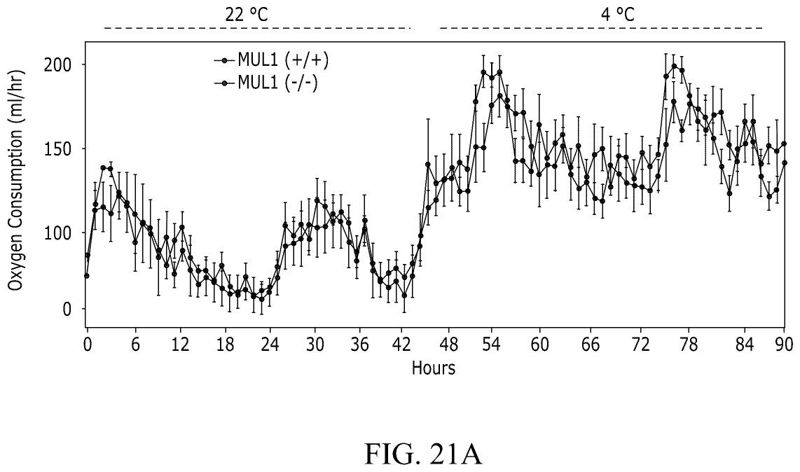

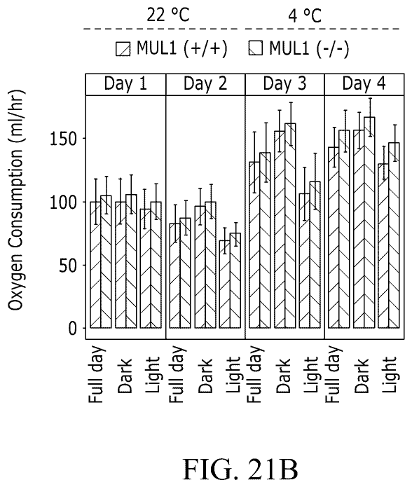

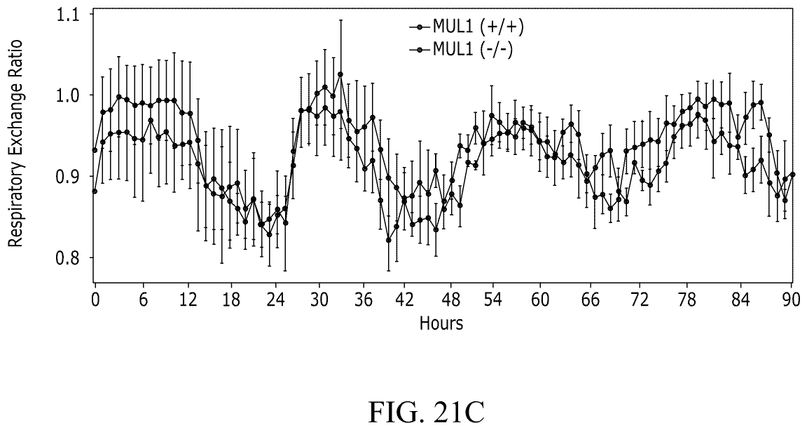

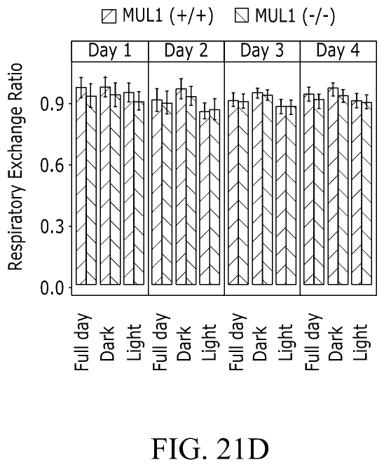

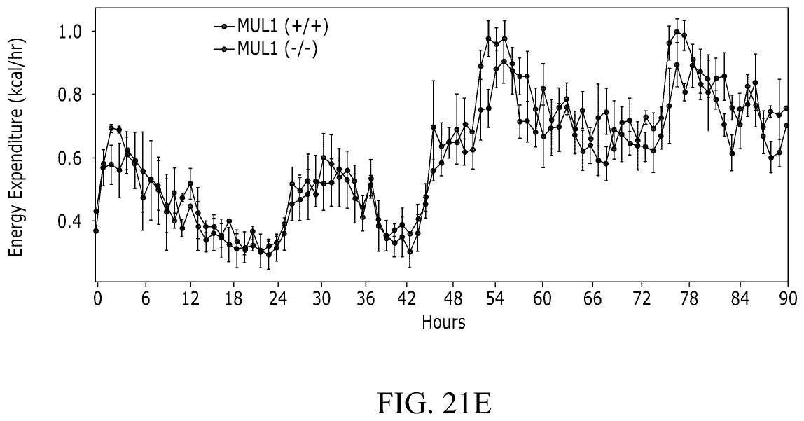

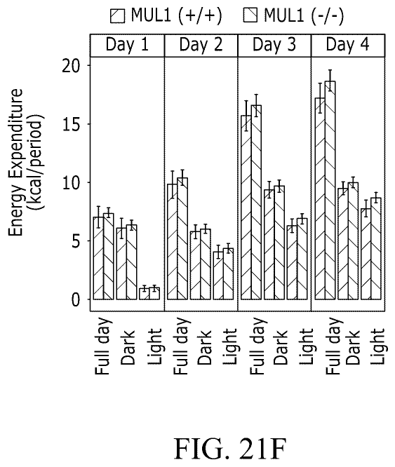

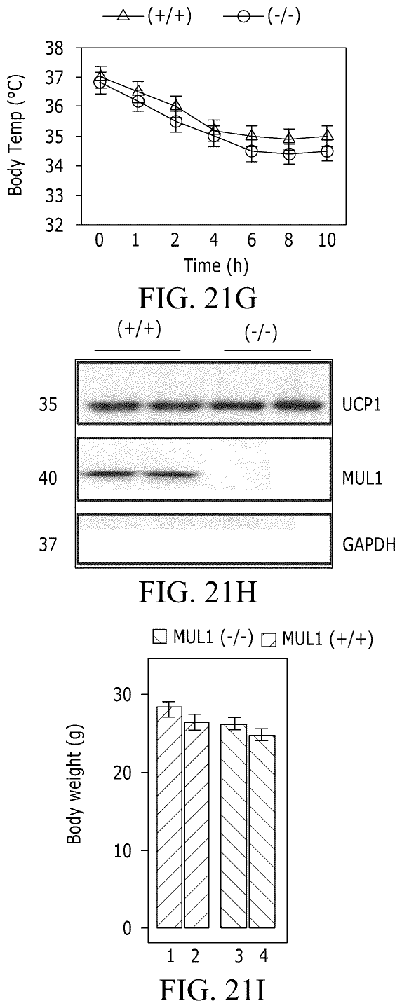

Mul1(−/−) mice have intact thermoregulation—Any potential role Mul1 inactivation might have in thermoregulation using Mul1(−/−) and Mul1(+/+) mice on a ND was explored. The two groups of animals were kept at the ambient temperature of 22° C. for 48 hours and then exposed to acute cold-challenged for another 48 hours at 4° C. Under these conditions, the oxygen consumption rate (FIGS. 21A-B), the respiratory exchange ratio (FIGS. 21C-D), and the energy expenditure (FIGS. 21E-F) were similar between the Mul1(−/−) and Mul1 (+/+) mice. Additionally, the body temperature of the animals kept at 4° C. for up to 10 hours was monitored. Mul1(−/−) animals had intact thermoregulation and were able to cold-adapt with no significant difference compared Mul1(+/+) (FIG. 21G). Consistent with these observations, there was no detectable change in the expression of the uncoupling protein 1 (UCP1) in the brown adipose tissue (BAT) between Mul1(−/−) and Mul1(+/+) animals, following the cold challenge (FIG. 21H). FIG. 21I shows the average weight of the animals before and after the cold challenge (n=4).

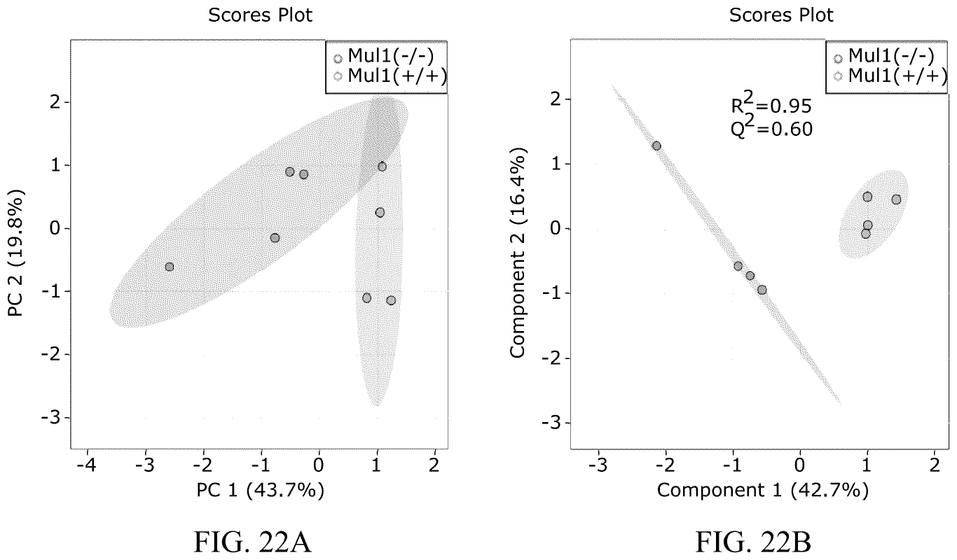

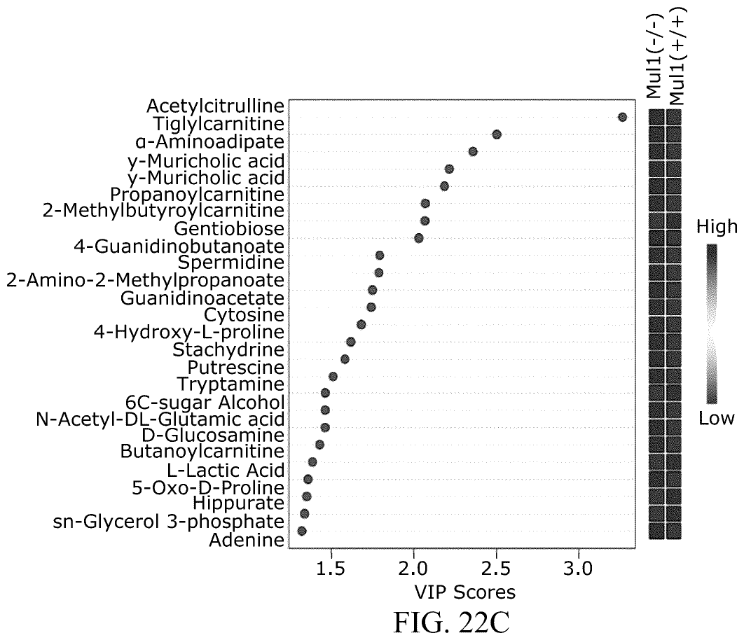

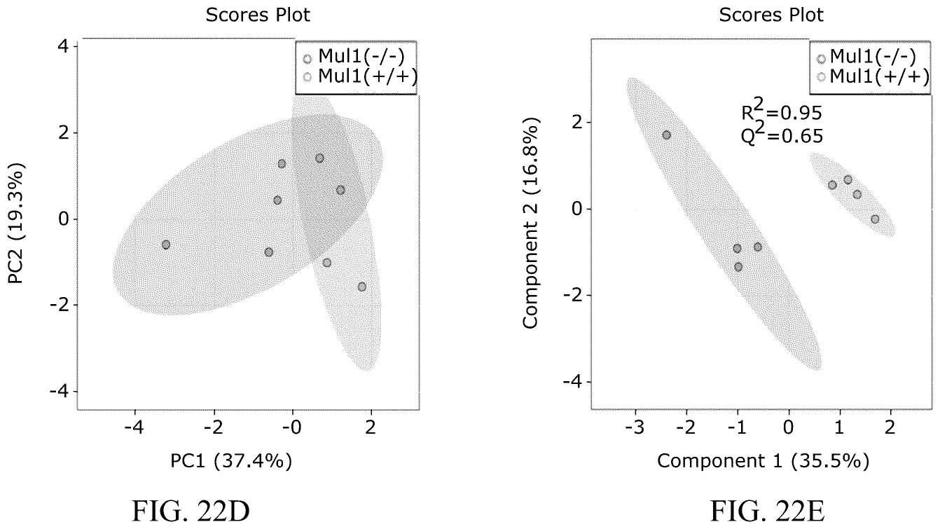

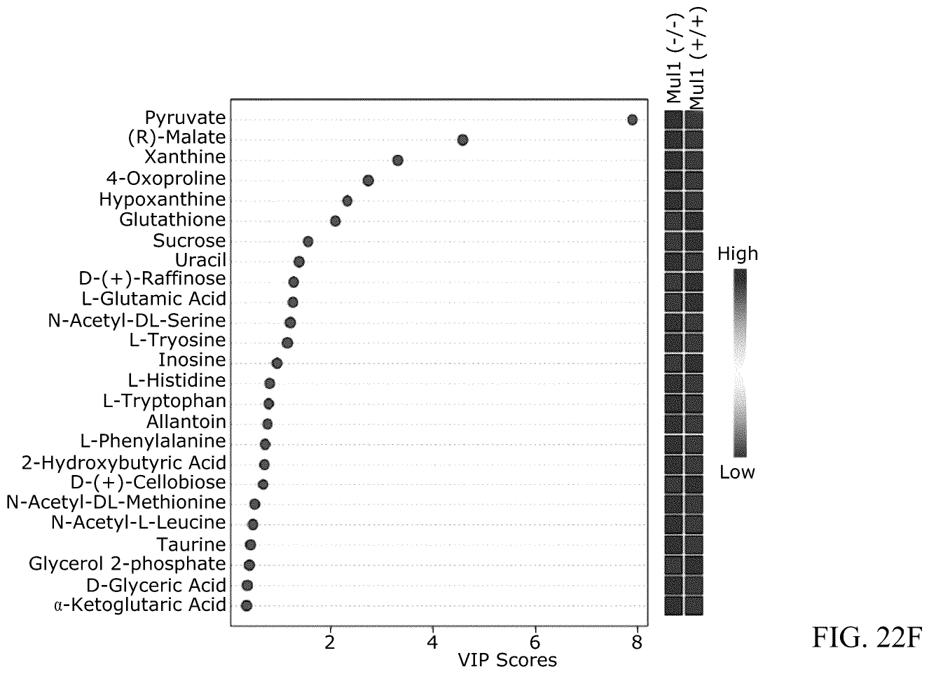

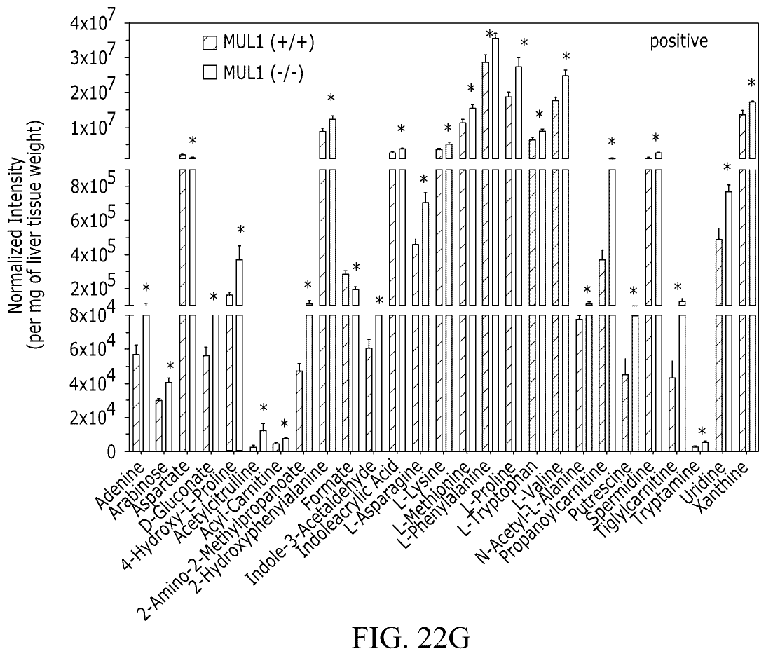

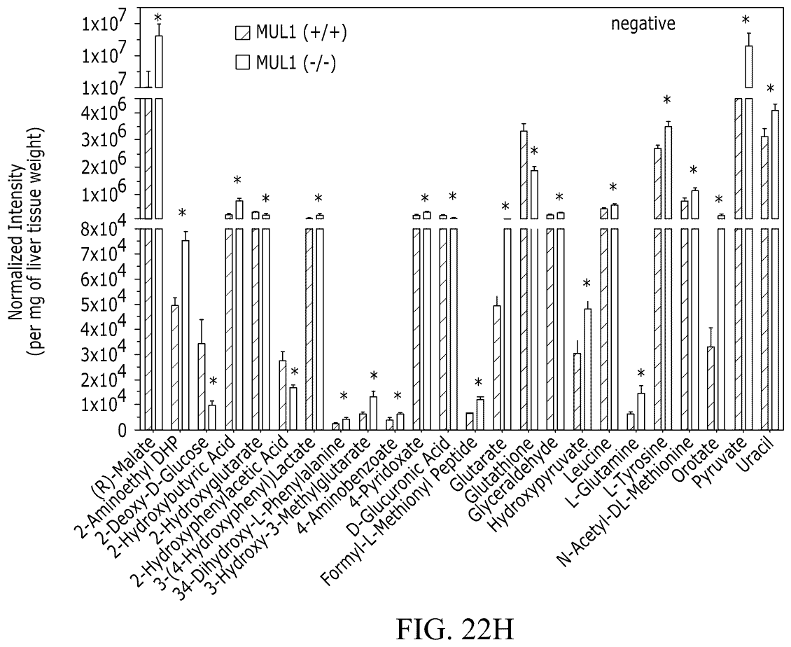

Global metabolomic analysis of Mul1(+/+) and Mul1(−/−) mouse liver on HFD—The effect of the Mul1 inactivation on the metabolic homeostasis in the liver of mice on HFD was investigated using LC-MS global metabolomics. The unsupervised principal component analysis (PCA) scores plots show clear separation in the metabolic profile between Mul1(+/+) and Mul1(−/−) HFD-liver in LC-MS positive ion mode (FIGS. 22A-B). The supervised partial least squares discriminant analysis (PLS-DA) model was used to predict the classes of samples and maximize the separation between groups. The PLS-DA variable importance projection (VIP) scores plot from LC-MS positive mode identified a large group of metabolites that showed differences between Mul1(+/+) and Mul1(−/−) HFD-liver (FIG. 22C). The VIP scores plot identified metabolites such as acetylcitrulline, a-aminoadipate, propanoylcarnitine, adenine, D-glucosamine, cytosine, and guanidinoacetate, which were found to be significantly higher in the liver of Mul1(−/−) on HFD. Whereas, lactic acid, hippurate, 6C sugar alcohol, and glycerol 3-phosphate were lower in Mul1(−/−) liver. Similarly, LC-MS negative mode data also showed separation in PCA and PLS-DA models (FIGS. 22D-E). The PLS-DA VIP scores plot showed that the metabolites such as pyruvate, malate, histidine, xanthine, tryptophan were found higher in Mul1(−/−) HFD-liver than Mul1(+/+). Metabolites such as glutathione, sucrose, raffinose, and glycerol 2-phosphate were found higher in Mul1(+/+) (FIG. 22F). The semiquantitative readouts of the metabolite levels demonstrate that large numbers of significantly different metabolites that drive the PLS-DA classifications (FIGS. 22G-H).

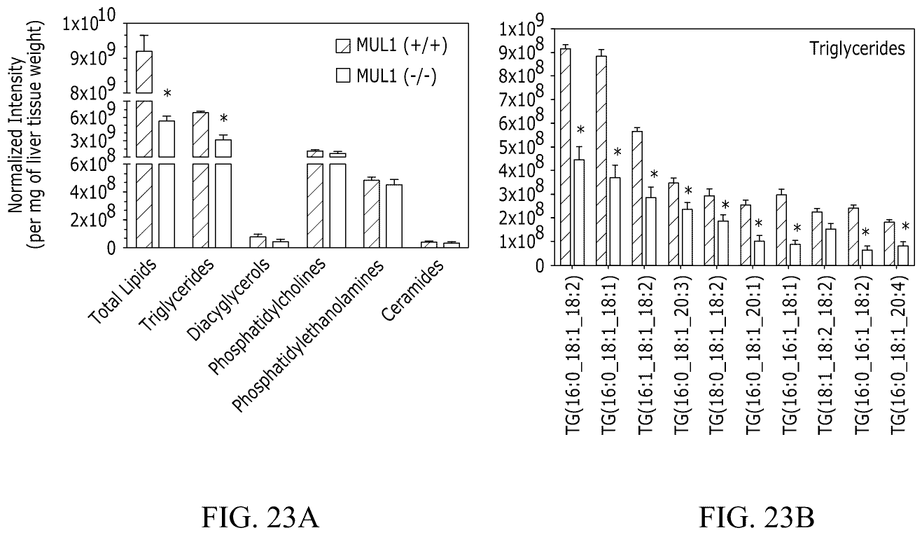

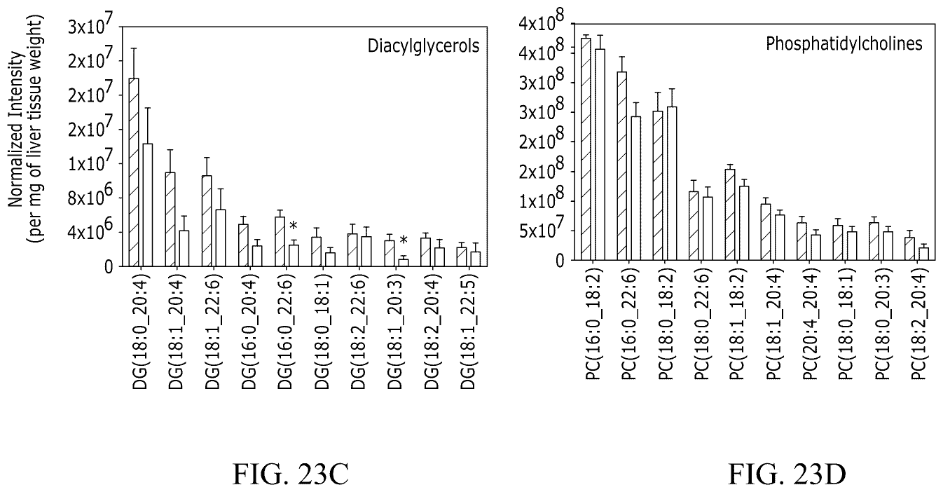

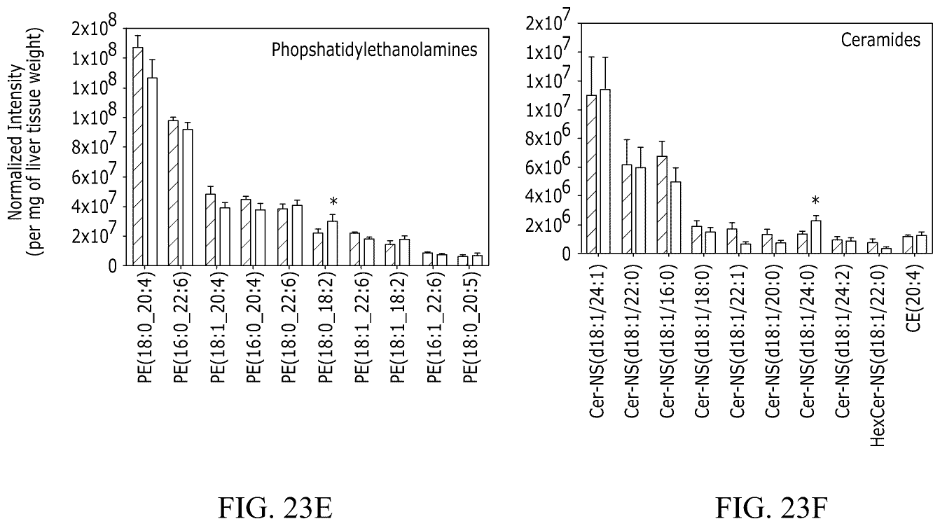

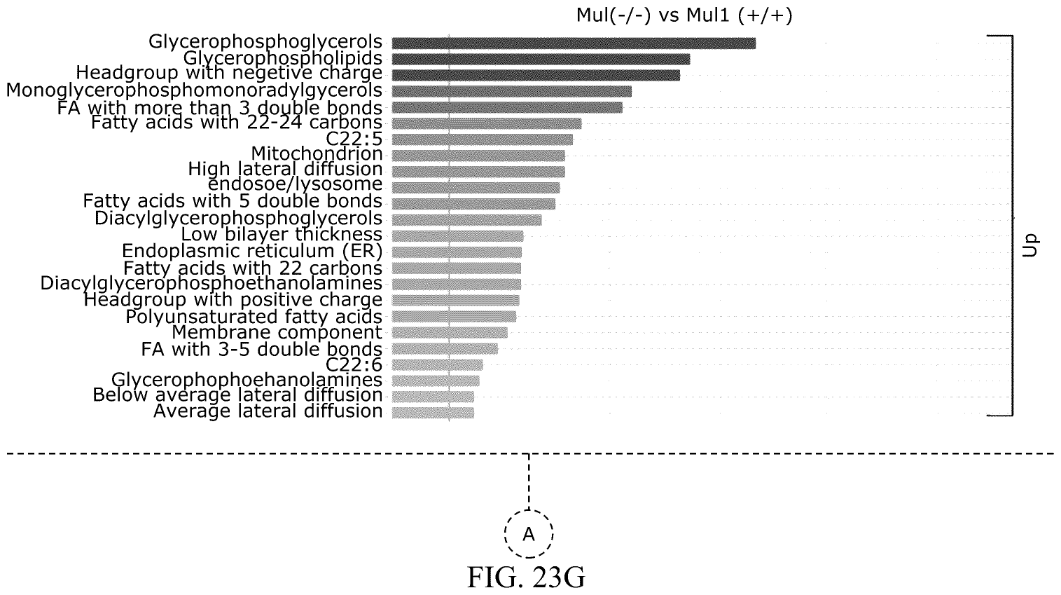

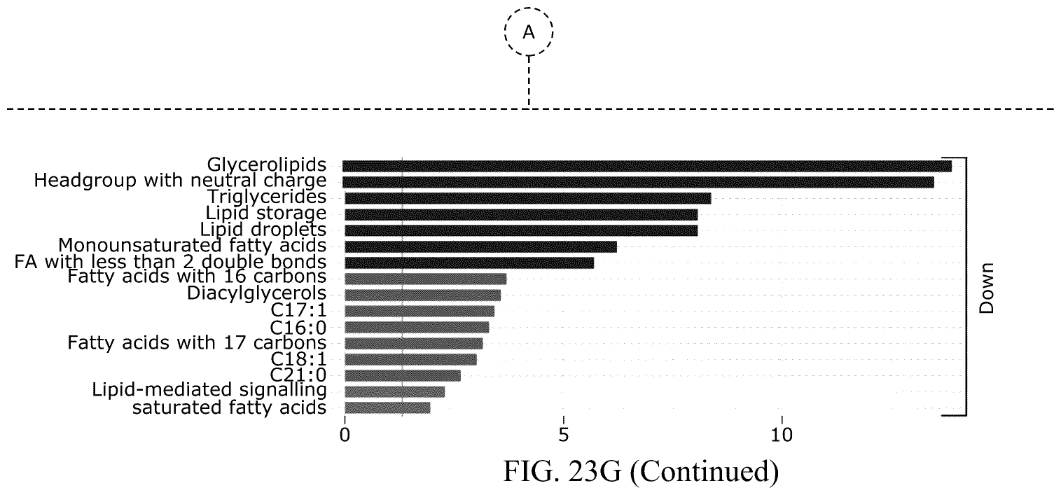

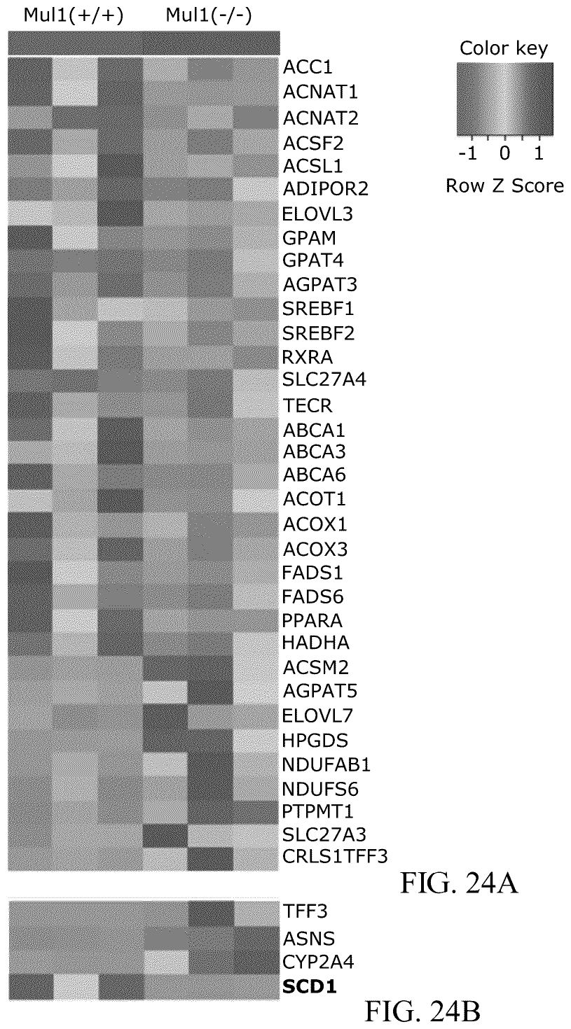

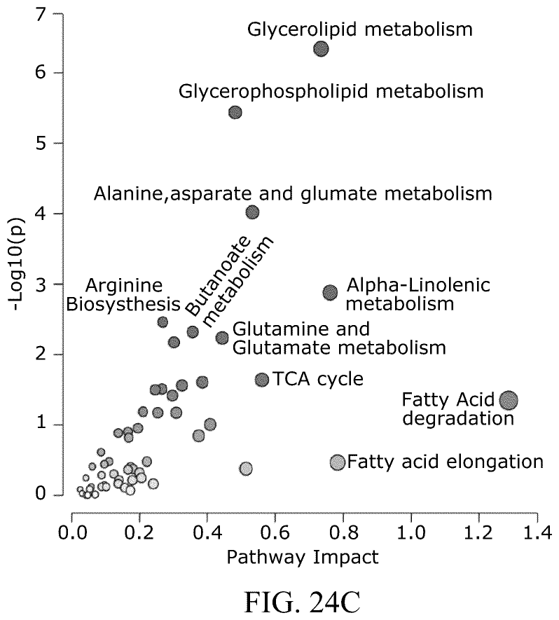

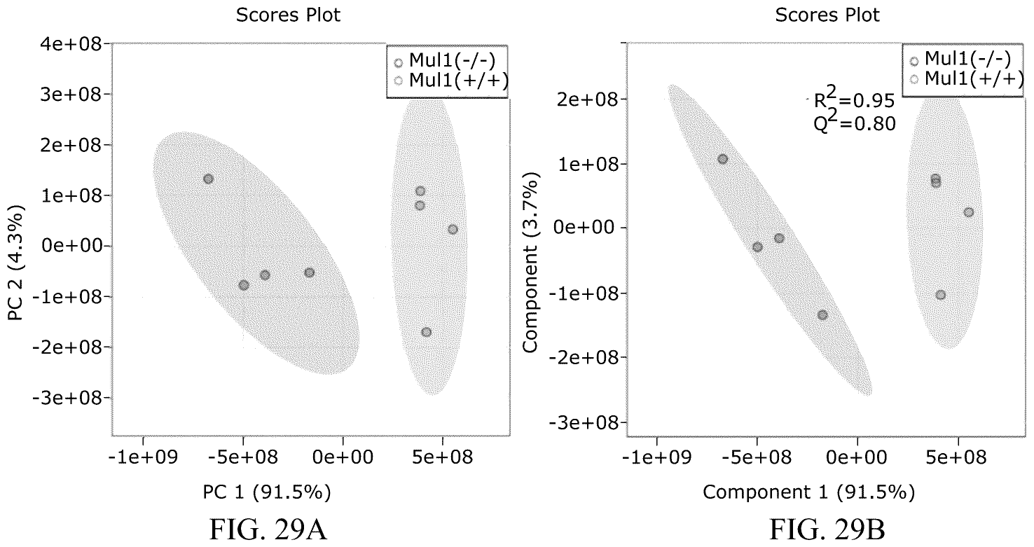

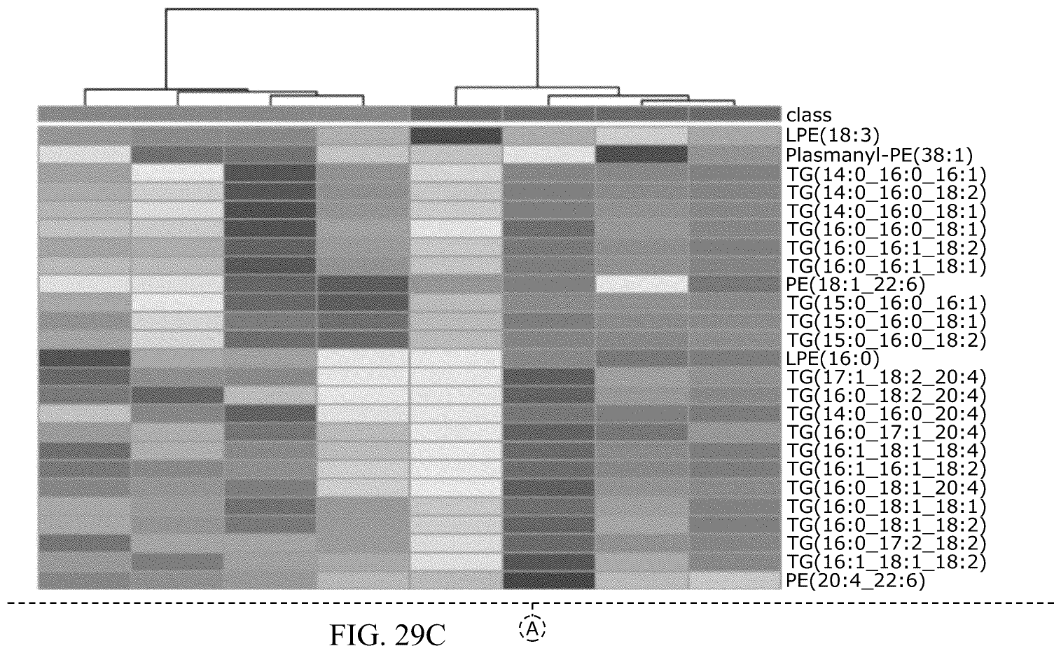

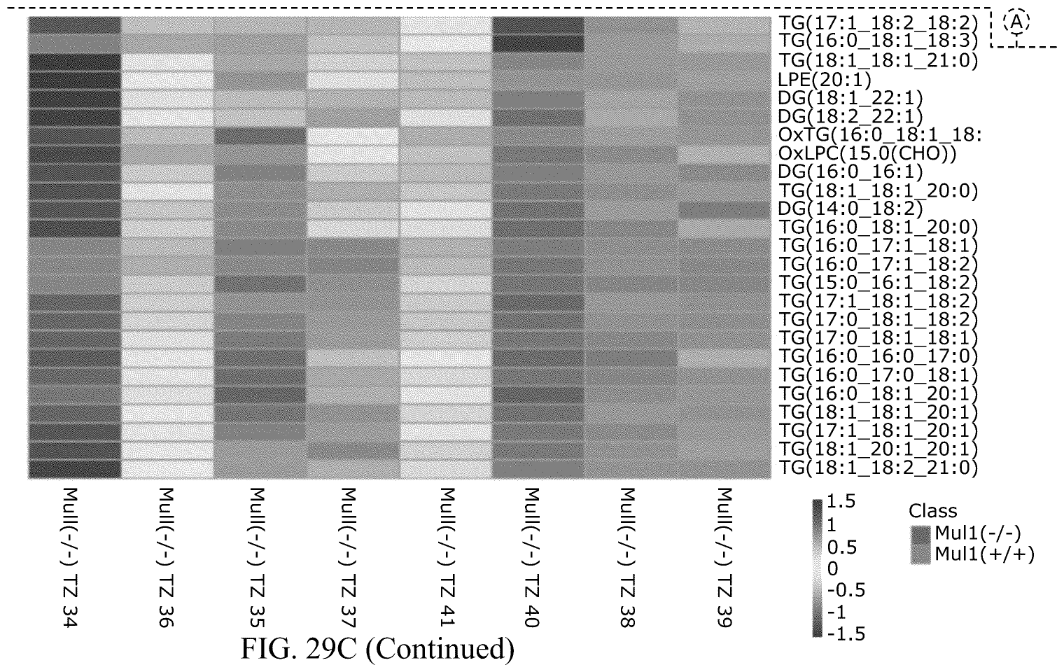

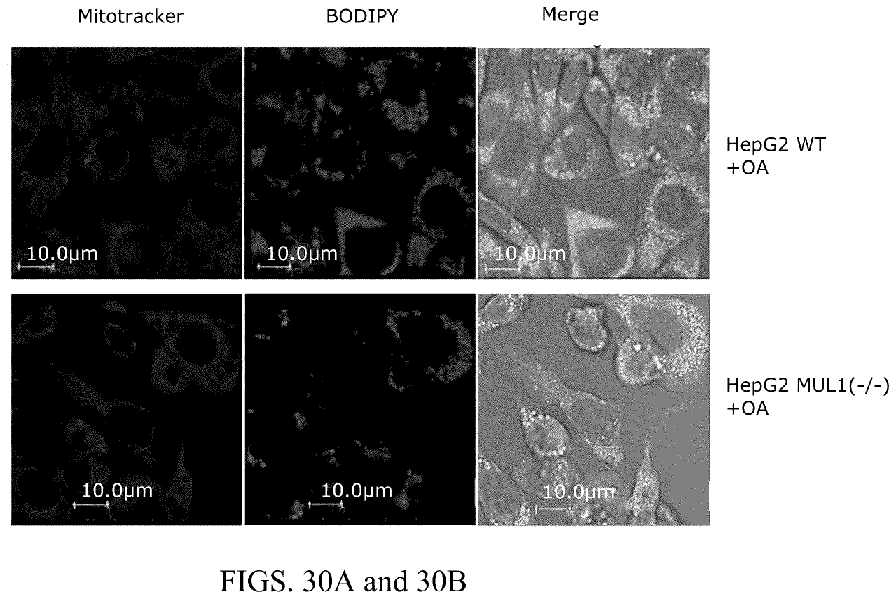

Liver lipidomic profiles of Mul1(+/+) and Mul1(−/−) mice on HFD—The total lipid content and profiling of lipid classes was carried out using the normalized intensities by liver weight (FIG. 23A). As expected, the total lipid content and triglycerides were found to be significantly higher in the liver of Mul1(+/+) than Mul1(−/−) mice on HFD. Total diacylglycerol, phosphatidylcholines, phosphatidylethanolamines, and ceramides were not significantly different between the two groups of animals (FIG. 23A). Nine of the top 10 triglycerides by abundance were significantly different between both groups, and higher in Mul1(+/+) HFD-liver (FIG. 23B). DG (16:0_22:6) and DG (18:1_20:3) were significantly higher in Mul1(+/+) HFD-liver than Mul1(−/−) HFD-liver (FIG. 23C). Other classes of lipids showed fewer significant differences (FIGS. 23D-F). LION term enrichment analysis of Mul1(+/+) versus Mul1(−/−) liver summarized the changes in lipid entities in Mul1(−/−) HFD liver (FIG. 23G). Glycerophosphoglycerols, glycerophospholipids, and long-chain fatty acids (C22-C24) were significantly higher in Mul1(−/−) HFD liver (FIG. 24C). The joint-metabolic pathway analysis also showed that glycerophospholipid metabolism is significantly altered in Mul1(−/−) HFD liver. The lipids containing neutral head groups, triglycerides, lipid storage, and glycerolipids were found to be significantly lower in Mul1(−/−) HFD liver. The lipidomic analysis indicates that MUL1 inactivation causes a massive change in lipid metabolism even with the supplementation of HFD diet. FIG. 23B clearly indicates that monounsaturated and polyunsaturated fatty acyl containing triglycerides are significantly lower in Mul1(−/−) HFD liver. FIG. 23G shows that monounsaturated fatty acids, triglycerides, lipid droplets, and lipid storage as integrated pathways were downregulated in Mul1(−/−) HFD-liver including palmitoleate and oleate, products of SCD1 activity. Additionally, PCA and PLS-DA analysis as well as clustered-heatmap clearly showed the separation in the overall lipid profiles between Mul1(−/−) and Mul1(+/+) HFD-liver (FIGS. 29A-B and FIG. 28C).

Differential gene expression in the liver of Mul1(−/−) mice on HFD—mRNA sequencing identified about 5,000 differentially regulated genes (p value <0.027) in the HFD-liver between Mul1(−/−) and Mul1(+/+) animals. The heatmap in FIG. 24A highlights genes involved in fatty acid biosynthesis, and metabolism that were significantly downregulated in the liver of Mul1(−/−) mice on HFD. The few genes unregulated are involved in mitochondrial function and include the mitochondrial cardiolipin synthase 1 (CRLS1), and protein tyrosine phosphatase mitochondrial 1 (PTPMT1). Both proteins are involved in the synthesis of cardiolipin (CL) and CRLS1 overexpression has been previously shown to ameliorate, lipid accumulation, hepatic steatosis, and insulin resistance [191]. There is also increased expression of Hematopoietic Prostaglandin D Synthase (HPGDS) involved in prostaglandin synthesis and immune response [192]. In addition, there is upregulation of NADH:ubiquinone oxidoreductase subunit S6 (NDUFS6), and NADH:ubiquinone oxidoreductase subunit AB1 (NDUFAB1) which are both components of the respiratory chain Complex I, involved in the electron transport. The top four ranked differentially regulated genes are shown in FIG. 24B and represent enzymes with diverse metabolic function. Trefoil factor 3 (Tff3), a small mainly secreted peptide, which when overexpressed in liver has been shown to increase fatty acid oxidation and inhibit gluconeogenesis [193,194]. Asparagine synthetase (Asns), a cytoplasmic enzyme that generates asparagine from aspartate. Cyp2a4, a member of the mammalian Cytochrome P450 (Cyp) gene superfamily that participates in a wide range of metabolic processes in the liver. And finally, Stearoyl-CoA Desaturase 1 (SCD1) which controls the biosynthesis of monounsaturated fatty acids (MUFA) from their saturated fatty acid (SFA) precursors, introducing a cis-double bond at the 49 position to stearoyl (C18:0) and palmitoyl-CoA (C16:0). SCD1 is a key regulator in lipid metabolism; high expression of SCD1 protein correlates with obesity, diabetes, and atherosclerosis and inactivation of SCD1 supports an HFD-resistant phenotype, reduced fat accumulation and insulin sensitivity [178,195-198]. These studies are focused on SCD1 as the potential mediator of MUL1 function in lipid metabolism since SCD1 is strongly downregulated in the absence of MUL1 and that the Scd1(−/−) mouse phenotype closely resembles that of the Mul1(−/−) animals [178-181]. In addition, a joint metabolic pathway analysis using uniport protein ID and HMDB ID of genes and metabolites, respectively, was performed. The fold change of genes and metabolite levels between the Mul1(−/−) and Mul1(+/+) HFD-liver were used as an input to create an integrative metabolic pathway analysis. (FIG. 24C). The results from joint pathway analysis depicted in FIG. 24B indicate that glycerolipids, glycerophospholipid, fatty acid metabolism, and alanine-aspartate-glutamate metabolism, were significantly affected in Mul1(−/−) HFD liver.

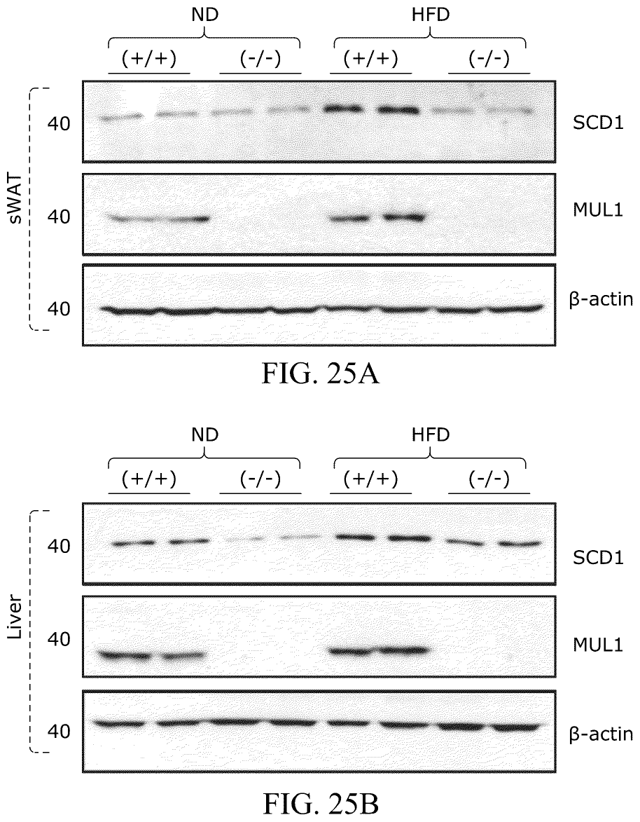

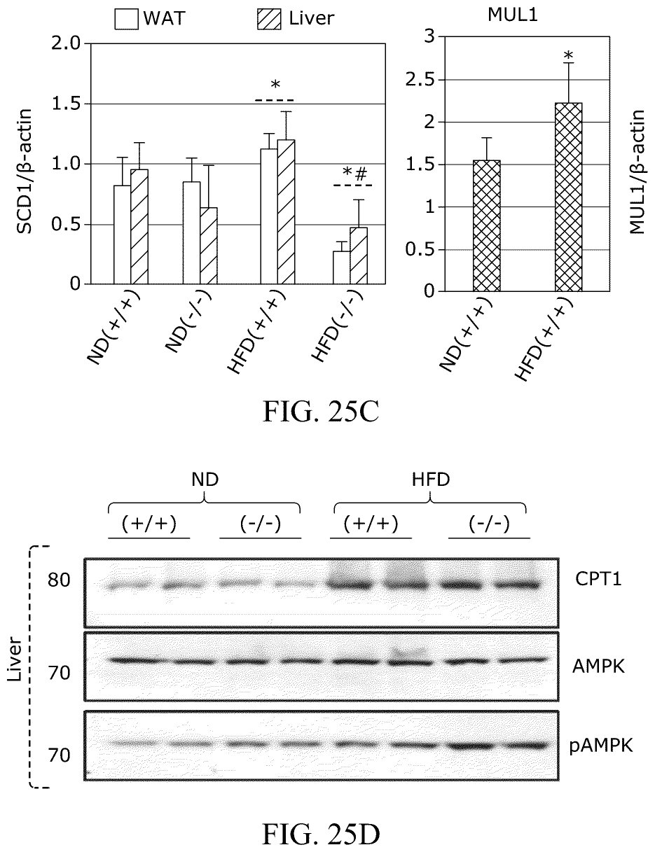

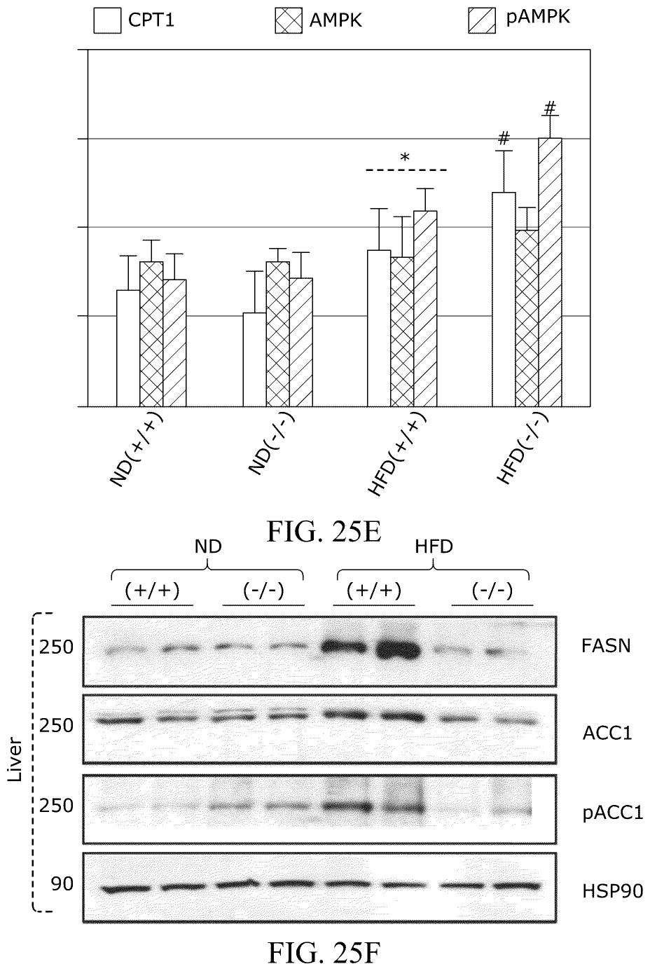

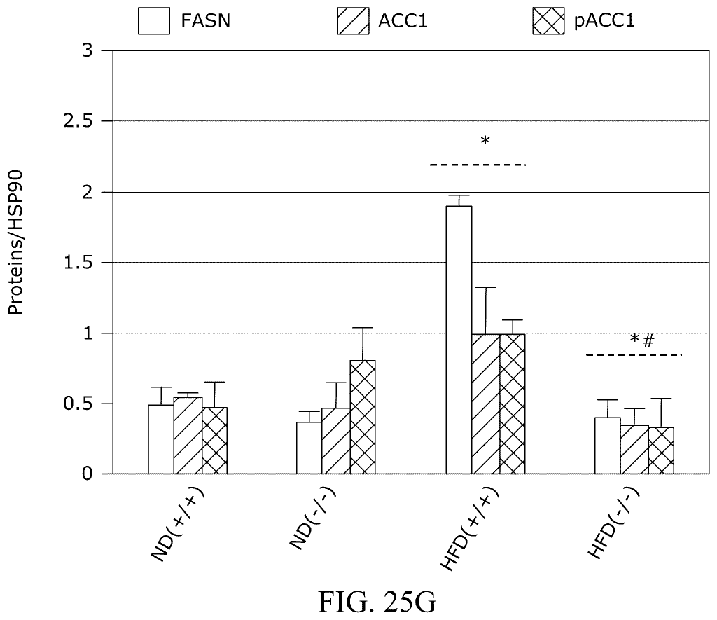

Expression and regulation of SCD1 pathway in lipogenesis in the absence of Mul1—The protein expression level of various enzymes that are known to be involved in the SCD1 pathway and the regulation of lipogenesis and b-oxidation was investigated. FIG. 25A shows that there was no significant difference in the expression of SCD1 in the sWAT from Mul1(−/−) mice maintained on a ND. However, there is a significant induction of SCD1 in Mul1(+/+) animals on HFD and this induction is inhibited in Mul1(−/−) under the same conditions. In the Mul1(−/−) liver there is a significant SCD1 downregulation in both ND and HFD compared to Mul1(+/+) animals (FIG. 25B). Expression of MUL1 and SCD1 is induced by HFD in the sWAT and liver of Mul1(+/+) mice (FIGS. 25A-B). FIG. 25C is a graph representation of densitometric analysis of the proteins from FIGS. 25A-B normalized against b-actin. CPT1 protein increased with HFD in both groups but Mul1(−/−) mice expressed higher level of the protein (FIGS. 25D-E). AMPK protein level is not affected by the diet or presence of MUL1, however the phospho-AMPK (pAMPK) which represents the active form of the enzyme, is substantially higher in Mul1(−/−) mice on HFD (FIGS. 25D-E). FASN, ACC1 and its phosphorylated form pACC1 are also strongly induced with HFD and this induction is completely absent in Mul1(−/−) HFD-liver (FIGS. 25F-G). HSP90 protein expression was used as a loading control (FIG. 25F). The pattern of expression of all these proteins during ND or HFD suggests that MUL1 is upstream of SCD1. Furthermore, the regulation of this pathway in the absence of MUL1 occurs primary during HFD. This result suggests a unique function for MUL1 in lipogenesis and metabolism during conditions of nutritional overload such as in HFD.

### Discussion of Experimentation—Study Two

MUL1 is a unique E3 ubiquitin ligase due to its exclusive localization in the outer mitochondrial membrane and its ability to modify specific substrates through SUMOylation, as well as K63- or K48-ubiquitination [23,25,26,40,199]. In addition, MUL1 has a large intermembrane domain (IMD) that can act as a sensor of various conditions in the mitochondria and modulate its ligase activity against various substrates [24,167]. The IMD of MUL1 is also the target of mitochondrial protease Omi/HtrA2 that is involved in protein quality control and can regulate MUL1 protein levels during mitochondrial stress. [22,174]. The studies disclosed herein, using established human cell lines, indicated that the absence of MUL1 protein expression could support a unique metabolic phenotype [174].

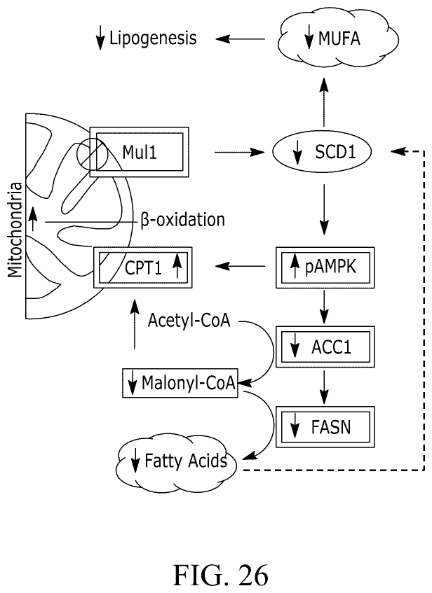

The function of mitochondrial MUL1 E3 ubiquitin ligase in the regulation of lipid metabolism under conditions of nutritional overload in the form of a high fat diet was investigated. It was found that animals with whole body Mul1 inactivation are resistant to HFD-induced obesity, liver steatosis, insulin insensitivity, and glucose intolerance. In addition, Mul1(−/−) animals have a hypermetabolic phenotype, significantly higher oxygen consumption, and energy expenditure but an intact thermoregulation when compared to their Mul1(+/+) littermates. Through metabolomic and lipidomic studies of mouse livers from Mul1(−/−) and Mul1(+/+) animals on HFD, several clusters of genes involved in both fatty acid oxidation and lipogenesis were identified to be differentially expressed. One of the top genes, whose mRNA expression is dramatically downregulated in the absence of MUL1, was SCD1. SCD1 protein is located exclusively in the endoplasmic reticulum (ER) where it is anchored through four transmembrane domains and exposing both catalytic amino and carboxy-terminal domains to the cytoplasm [200]. SCD1 is a rate-limiting enzyme that catalyzes the biosynthesis of monounsaturated fatty acids (MUFA) from palmitate and stearate, their saturated fatty acids (SFA) precursors [180,197,201]. A plethora of previous studies have established SCD1 as a key regulator in lipid metabolism; high expression of SCD1 protein correlates with obesity, diabetes, and atherosclerosis and inactivation of Scd1 gene supports an HFD-resistant phenotype, reduced fat accumulation and insulin sensitivity [178-181]. The studies disclosed herein are focused on SCD1 enzyme as a potential downstream mediator of MUL1's function in lipogenesis since mice with SCD1 inactivation have a phenotype that closely resembles that of Mul1(−/−) animals. This phenotype includes increased fatty acid oxidation, attenuated lipid accumulation, and resistance to HFD-induced obesity [202-204]. SCD1 is expressed predominantly in the liver and white adipose tissue, where its protein expression is induced by HFD. In Mul1(−/−) mice the SCD1 expression in adipose tissue and liver is markedly downregulated particularly during HFD conditions. Since Mul1(−/−) animals have increased β-oxidation and reduced lipogenesis, the expression of key enzymes involved in both processes was monitored. It was found that MUL1 deficiency activates AMPK by increasing its phosphorylation (pAMPK), which in turn drives a cascade of reactions involved in cellular energy homeostasis. pAMPK promotes the expression and accumulation of its downstream target CPT1, that is in the OMM, and facilitates the import of fatty acids to drive b-oxidation [202]. In addition, activation of pAMPK in the liver of Mul1(−/−) mice leads to the downregulation ACC1 and FASN, enzymes involved in fatty acid synthesis. Most of these enzymes have also been shown to be regulated in a similar manner in the liver of the Scd1(−/−) animals [202,205,206]. FIG. 26 is a schematic diagram of the proposed MUL1-regulated pathway in lipogenesis and b-oxidation in the liver of mice on HFD. It is interesting to note that the absence of MUL1 function does not affect the expression of all these proteins in the liver of animals fed a regular chow diet, but it has a dramatic effect in reducing their expression or induction that is associated with HFD. The data suggests that Scd1 gene is the main mediator of MUL1's function in lipogenesis and acts as a modulator of lipid metabolism and energy homeostasis especially during conditions of nutritional overload in the form of HFD. There are several other proteins regulated in the liver of Mul1(−/−) mice on HFD, that could potentially play a supporting role in the metabolic phenotype of these mice. These proteins might be downstream targets of SCD1, or they could be regulated directly in the absence of Mul1. The mechanism by which MUL1 inactivation suppresses the expression of SCD1 in liver and fat tissue, especially under conditions of HFD, is unclear. The mRNA sequencing identified Sterol Regulatory Element-Binding Protein 1 (SREBP1) and Retinoid X Receptor (RXR) transcriptional activators involved in Scd1 transcriptional control [207,208] to be down regulated in Mul1(−/−) animals. MUL1 protein is expressed ubiquitously, and the mice used herein carry a whole-body inactivation of Mul1 gene. Since the effect of MUL1 in the regulation of SCD1 was seen primarily in the liver and sWAT, it will be of interest to investigate if liver and/or fat tissue specific Mul1 deletion alone can replicate a similar metabolic phenotype. In addition, it is possible MUL1 has different tissue specific functions beyond its role in lipogenesis in liver and sWAT. The biological function of MUL1 in mitochondrial dynamics and more specifically in mitophagy has been often compared with that of Parkin. Both MUL1 and Parkin are E3 ubiquitin ligases, MUL1 is mito-resident whereas Parkin is mito-recruited. Several reports have shown MUL1 can work in parallel or in synergy, independent, or opposite, to Parkin's role in mitophagy [37,209-211], The study described herein indicates that the similarities between MUL1 and Parkin function also extend in the regulation of adiposity. Recent studies have shown Parkin inactivation in the adipose tissue alleviates HFD-induced obesity as well as aging-induced adiposity in mice [212.213]. The study described herein demonstrates that the molecular mechanisms of MUL1 function in lipogenesis is different from the one reported for Parkin [213]. It will be of interest to investigate if there is any crosstalk between these two E3 ligases or if they work independently to regulate lipogenesis. In summary, the study described herein establishes a new function for mitochondrial MUL1 E3-ubiquitin ligase in the regulation of lipogenesis and fatty acid oxidation, particularly during conditions of HFD. The data also suggests that MUL1 can be a very promising target for the development of chemical molecule inhibitors or therapeutic siRNA that could be used against obesity and the accompanying metabolic disease.

### CONCLUSION

### Summary of Experimental Findings: The Role of Mitochondrial MUL1 E3 Ligase in the Regulation of Metabolism and Obesity

The instant inventors have identified a new molecular pathway, mediated by the MUL1 E3 ubiquitin ligase, that regulates mitochondrial metabolism. MUL1 is a multifunctional E3 ubiquitin ligase that participates in various biological pathways, including mitochondrial dynamics, apoptosis, and innate immune response. It was discovered that MUL1 can regulate HIF-1α protein levels under aerobic conditions. Inactivation of MUL1 causes accumulation of HIF-1α, as well as Akt2, ULK1, and Mfn2 proteins, which are known to be involved in mitochondrial dynamics and metabolism. MUL1 inactivation causes a metabolic shift from OXPHOS to glycolysis, a phenotype resembling the Warburg effect, a phenomenon associated with most cancer cells where glycolysis is predominantly used as the main source of ATP production. Recent studies suggest that aerobic glycolysis is not only the hallmark of cancer, but it is also involved in many physiological functions. These include embryogenesis, stem cell differentiation, innate and adaptive immunity, type 2 diabetes, starvation, as well as cardiomyopathy. Mice carrying a global inactivation of the Mul1 gene have a very distinct metabolic phenotype and they are resistant to HFD-induced obesity. The regulation of mitochondrial metabolism by MUL1, especially its role in fat oxidation and obesity has not been previously investigated, which is the primary focus of these studies. It is thought that loss of MUL1 alters mitochondrial dynamics, electron transport and oxidative metabolism. Therefore, the respective mechanism and contribution of HIF-1α, Akt2, ULK1, and Mfn2 proteins to the MUL1 mediated metabolic phenotype have been investigated. In addition, the obesity resistant phenotype of the Mul1(−/−) mice has been investigated in detail using animals fed with either regular chow or HFD. Whole body metabolism was monitored using the “Promethion” metabolic cages system” and state-of-the-art techniques including global LC-MS metabolomics, lipidomic, mRNA sequencing, and isotope-based flux measurements, all of which are sensitive to modified glycolysis and TCA turnover in Mul1(−/−) mice. In addition, temperature homeostasis, in Mul1(−/−) mice was studied to verify if the mice can maintain optimal body temperature (37° C.) when exposed to acute (4° C.) and or gradual cold challenge, a condition that requires close functional coordination between thermogenic and metabolic organs.

A combination of approaches to elucidate the molecular mechanism of a new pathway that drives mitochondrial metabolism and makes the knockout mouse resistant to HFD-induced obesity were used in the studies described herein. The role of the several key proteins in this pathway were characterized and the involvement of mitochondria dynamics was monitored. A new function of the mitochondrial Mul1 E3 ubiquitin ligase in the regulation of lipogenesis and adiposity, particularly during conditions of a high fat diet (HFD), was identified. Inactivation of Mul1 provides resistance to HFD-induced obesity and can be a therapeutic target for treatment of obesity. It is contemplated that these studies could lead to the development of pharmacological inhibitors of MUL1 ligase for prevention and treatment of obesity in humans, providing protection against nonalcoholic fatty liver disease (NAFLD), type 2 diabetes, heart or kidney disease, stroke, and certain kinds of cancers.

All patents and publications mentioned in this specification are indicative of the levels of those skilled in the art to which the invention pertains. All patents and publications are herein incorporated by reference to the same extent as if each individual publication were specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is adapted to carry out the objectives and obtain the ends and advantages mentioned, as well as those inherent therein. The regulators, modulators, inhibitors, compositions, genetic profiles, kits, and methods described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention, which are obvious to those skilled in the art, are intended to be within the scope of the invention.

### REFERENCE LIST

1. Castellanos E, Lanning N J. Phosphorylation of OXPHOS Machinery Subunits: Functional Implications in Cell Biology and Disease. Yale J Biol Med. 2019; 92(3):523-31.
2. Kim J W, Tchernyshyov I, Semenza G L, Dang C V. HIF-1-mediated expression of pyruvate dehydrogenase kinase: a metabolic switch required for cellular adaptation to hypoxia. Cell Metab. 2006; 3(3):177-85.
3. Semenza G L. HIF-1 mediates metabolic responses to intratumoral hypoxia and oncogenic mutations. J Clin Invest. 2013; 123(9):3664-71.
4. Yee Koh M, Spivak-Kroizman T R, Powis G. HIF-1 regulation: not so easy come, easy go. Trends Biochem Sci. 2008; 33(11):526-34.
5. Suzuki N, Gradin K, Poellinger L, Yamamoto M. Regulation of hypoxia-inducible gene expression after HIF activation. Exp Cell Res. 2017; 356(2):182-6.
6. Wenger R H, Stiehl D P, Camenisch G. Integration of oxygen signaling at the consensus HRE. Sci STKE. 2005; 2005(306):re12.
7. Marin-Hernandez A, Gallardo-Perez J C, Ralph S J, Rodriguez-Enriquez S, Moreno-Sanchez R. HIF-1alpha modulates energy metabolism in cancer cells by inducing over-expression of specific glycolytic isoforms. Mini Rev Med Chem. 2009; 9(9):1084-101.
8. Stockmann C, Fandrey J. Hypoxia-induced erythropoietin production: a paradigm for oxygen-regulated gene expression. Clin Exp Pharmacol Physiol. 2006; 33(10):968-79.
9. Cerychova R, Pavlinkova G. HIF-1, Metabolism, and Diabetes in the Embryonic and Adult

Heart. Front Endocrinol (Lausanne). 2018; 9:460.
10. Hui S, Ghergurovich J M, Morscher R J, Jang C, Teng X, Lu W, Esparza L A, Reya T, Le Z, Yanxiang Guo J, White E, Rabinowitz J D. Glucose feeds the TCA cycle via circulating lactate. Nature. 2017; 551(7678):115-8.
11. Menendez-Montes I, Escobar B, Palacios B, Gomez M J, Izquierdo-Garcia J L, Flores L, Jimenez-Borreguero L J, Aragones J, Ruiz-Cabello J, Tones M, Martin-Puig S. Myocardial VHL-HIF Signaling Controls an Embryonic Metabolic Switch Essential for Cardiac Maturation. Dev Cell. 2016; 39(6):724-39.
12. Palazon A, Goldrath A W, Nizet V, Johnson R S. HIF transcription factors, inflammation, and immunity. Immunity. 2014; 41(4):518-28.
13. Poznanski S M, Barra N G, Ashkar A A, Schertzer J D Immunometabolism of T cells and NK cells: metabolic control of effector and regulatory function. Inflamm Res. 2018; 67(10):813-28.
14. Warburg O. The Metabolism of Carcinoma Cells. The Journal of Cancer Research. 1925; 9(1):148-63.
15. Wu P, Sato J, Zhao Y, Jaskiewicz J, Popov K M, Harris R A. Starvation and diabetes increase the amount of pyruvate dehydrogenase kinase isoenzyme 4 in rat heart. Biochem J. 1998; 329 (Pt 1):197-201.
16. Allard M F, Schonekess B O, Henning S L, English D R, Lopaschuk G D. Contribution of oxidative metabolism and glycolysis to ATP production in hypertrophied hearts. Am J Physiol. 1994; 267(2 Pt 2):H742-50.
17. Lopaschuk G D, Spafford M A, Marsh D R. Glycolysis is predominant source of myocardial ATP production immediately after birth. Am J Physiol. 1991; 261(6 Pt 2):H1698-705.
18. Nishikawa T, Bellance N, Damm A, Bing H, Zhu Z, Handa K, Yovchev M I, Sehgal V, Moss T J, Oertel M, Ram P T, Pipinos, II, Soto-Gutierrez A, Fox I J, Nagrath D. A switch in the source of ATP production and a loss in capacity to perform glycolysis are hallmarks of hepatocyte failure in advance liver disease. J Hepatol. 2014; 60(6):1203-11.
19. Cardote T A F, Gadd M S, Ciulli A. Crystal Structure of the Cul2-Rbx1-EloBC-VHL Ubiquitin Ligase Complex. Structure. 2017; 25(6):901-11 e3.
20. Lin H C, Yeh C W, Chen Y F, Lee T T, Hsieh P Y, Rusnac D V, Lin S Y, Elledge S J, Zheng N, Yen H S. C-Terminal End-Directed Protein Elimination by CRL2 Ubiquitin Ligases. Mol Cell. 2018; 70(4):602-13 e3.
21. Kamura T, Brower C S, Conaway R C, Conaway J W. A molecular basis for stabilization of the von Hippel-Lindau (VHL) tumor suppressor protein by components of the VHL ubiquitin ligase. J Biol Chem. 2002; 277(33):30388-93.
22. Cilenti L, Di Gregorio J, Ambivero C T, Andl T, Liao R, Zervos A S. Mitochondrial MUL1 E3 ubiquitin ligase regulates Hypoxia Inducible Factor (HIF-1alpha) and metabolic reprogramming by modulating the UBXN7 cofactor protein. Sci Rep. 2020; 10(1):1609.
23. Braschi E, Zunino R, McBride H M. MAPL is a new mitochondrial SUMO E3 ligase that regulates mitochondrial fission. EMBO reports. 2009; 10(7):748-54.
24. Li W, Bengtson M H, Ulbrich A, Matsuda A, Reddy V A, Orth A, Chanda S K, Batalov S, Joazeiro C A. Genome-wide and functional annotation of human E3 ubiquitin ligases identifies MULAN, a mitochondrial E3 that regulates the organelle's dynamics and signaling. PloS one. 2008; 3(1):e1487.
25. Prudent J, Zunino R, Sugiura A, Mattie S, Shore G C, McBride H M. MAPL SUMOylation of Drp1 Stabilizes an ER/Mitochondrial Platform Required for Cell Death. Mol Cell. 2015; 59(6):941-55.
26. Ni G, Konno H, Barber G N. Ubiquitination of STING at lysine 224 controls IRF3 activation. Sci Immunol. 2017; 2(11).
27. Kim S Y, Kim H J, Kang S U, Kim Y E, Park J K, Shin Y S, Kim Y S, Lee K, Kim C H. Non-thermal plasma induces AKT degradation through turn-on the MUL1 E3 ligase in head and neck cancer. Oncotarget. 2015; 6(32):33382-96.
28. Bae S, Kim S Y, Jung J H, Yoon Y, Cha H J, Lee H, Kim K, Kim J, An I S, Um H D, Park I C, Lee S J, Nam S Y, Jin Y W, Lee J H, An S. Akt is negatively regulated by the MULAN E3 ligase. Cell research. 2012; 22(5):873-85.
29. Park Y Y, Lee S, Karbowski M, Neutzner A, Youle R J, Cho H. Loss of MARCH5 mitochondrial E3 ubiquitin ligase induces cellular senescence through dynamin-related protein 1 and mitofusin 1. Journal of cell science. 2010; 123(Pt 4):619-26.
30. Tang F, Wang B, Li N, Wu Y, Jia J, Suo T, Chen Q, Liu Y J, Tang J. RNF185, a novel mitochondrial ubiquitin E3 ligase, regulates autophagy through interaction with BNIP1. PloS one. 2011; 6(9):e24367.
31. Wang Q, Huang L, Hong Z, Lv Z, Mao Z, Tang Y, Kong X, Li S, Cui Y, Liu H, Zhang L, Zhang X, Jiang L, Wang C, Zhou Q. The E3 ubiquitin ligase RNF185 facilitates the cGAS-mediated innate immune response. PLoS Pathog. 2017; 13(3):e1006264.
32. Park Y J, Oanh N T K, Heo J, Kim S G, Lee H S, Lee H, Lee J H, Kang H C, Lim W, Yoo Y S, Cho H. Dual targeting of RIG-I and MAVS by MARCH5 mitochondria ubiquitin ligase in innate immunity. Cell Signal. 2020; 67:109520.
33. Kim S H, Park Y Y, Yoo Y S, Cho H. Self-clearance mechanism of mitochondrial E3 ligase MARCH5 contributes to mitochondria quality control. FEBS J. 2016; 283(2):294-304.
34. Di Gregorio J, Cilenti L, Ambivero C T, Andl T, Liao R, Zervos A S. UBXN7 cofactor of CRL3(KEAP1) and CRL2(VHL) ubiquitin ligase complexes mediates reciprocal regulation of NRF2 and HIF-1alpha proteins. Biochim Biophys Acta Mol Cell Res. 2021; 1868(4):118963.
35. Iso T, Suzuki T, Baird L, Yamamoto M. Absolute Amounts and Status of the Nrf2-Keap1-Cul3 Complex within Cells. Mol Cell Biol. 2016; 36(24):3100-12.
36. Tao S, Liu P, Luo G, Rojo de la Vega M, Chen H, Wu T, Tillotson J, Chapman E, Zhang D D. p97 Negatively Regulates NRF2 by Extracting Ubiquitylated NRF2 from the KEAP1-CUL3 E3 Complex. Mol Cell Biol. 2017; 37(8).
37. Yun J, Puri R, Yang H, Lizzio M A, Wu C, Sheng Z H, Guo M. MUL1 acts in parallel to the PINK1/parkin pathway in regulating mitofusin and compensates for loss of PINK1/parkin. Elife.

2014; 3:e01958.
38. Olszewska D A, Lynch T. MUL1-A new potential for a therapeutic target for Parkinson's disease a commentary on “MUL1 acts in parallel to the PINK1/parkin pathway in regulating mitofusin and compensates for loss of PINK1/parkin” by Yun and colleagues (eLife 2014; 3: 1-26). Mov Disord. 2016; 31(7):1037.
39. Li J, Qi W, Chen G, Feng D, Liu J, Ma B, Zhou C, Mu C, Zhang W, Chen Q, Zhu Y. Mitochondrial outer-membrane E3 ligase MUL1 ubiquitinates ULK1 and regulates selenite-induced mitophagy. Autophagy. 2015; 11(8):1216-29.
40. Kim S Y, Kim H J, Byeon H K, Kim D H, Kim C H. FOXO3 induces ubiquitylation of AKT through MUL1 regulation. Oncotarget. 2017; 8(66):110474-89.
41. Bouzakri K, Zachrisson A, Al-Khalili L, Zhang B B, Koistinen H A, Krook A, Zierath J R. siRNA-based gene silencing reveals specialized roles of IRS-1/Akt2 and IRS-2/Akt1 in glucose and lipid metabolism in human skeletal muscle. Cell Metab. 2006; 4(1):89-96.
42. Hedl M, Yan J, Abraham C. IRF5 and IRF5 Disease-Risk Variants Increase Glycolysis and Human M1 Macrophage Polarization by Regulating Proximal Signaling and Akt2 Activation. Cell Rep. 2016; 16(9):2442-55.
43. Li H, Lu S, Chen Y, Zheng L, Chen L, Ding H, Ding J, Lou D, Liu F, Zheng B. AKT2 phosphorylation of hexokinase 2 at T473 promotes tumorigenesis and metastasis in colon cancer cells via NF-kappaB, HIF1alpha, MMP2, and MMP9 upregulation. Cell Signal. 2019; 58:99-110.
44. Ng Y, Ramm G, Lopez J A, James D E. Rapid activation of Akt2 is sufficient to stimulate GLUT4 translocation in 3T3-L1 adipocytes. Cell Metab. 2008; 7(4):348-56.
45. Teng Y, Zhang Y, Qu K, Yang X, Fu J, Chen W, Li X. MicroRNA-29B (mir-29b) regulates the Warburg effect in ovarian cancer by targeting AKT2 and AKT3. Oncotarget. 2015; 6(38):40799-814.
46. Cleasby M E, Reinten T A, Cooney G J, James D E, Kraegen E W. Functional studies of Akt isoform specificity in skeletal muscle in vivo; maintained insulin sensitivity despite reduced insulin receptor substrate-1 expression. Mol Endocrinol. 2007; 21(1):215-28.
47. Lee R S, House C M, Cristiano B E, Hannan R D, Pearson R B, Hannan K M. Relative Expression Levels Rather Than Specific Activity Plays the Major Role in Determining In Vivo AKT Isoform Substrate Specificity. Enzyme Res. 2011; 2011:720985.
48. Wang J, Zhao W, Guo H, Fang Y, Stockman S E, Bai S, Ng P K, Li Y, Yu Q, Lu Y, Jeong K J, Chen X, Gao M, Liang J, Li W, Tian X, Jonasch E, Mills G B, Ding Z. AKT isoform-specific expression and activation across cancer lineages. BMC Cancer. 2018; 18(1):742.
49. Manning B D, Toker A. AKT/PKB Signaling: Navigating the Network. Cell. 2017; 169(3):381-405.
50. Dummler B, Hemmings B A. Physiological roles of PKB/Akt isoforms in development and disease. Biochem Soc Trans. 2007; 35(Pt 2):231-5.
51. Dummler B, Tschopp O, Hynx D, Yang Z Z, Dirnhofer S, Hemmings B A. Life with a single isoform of Akt: mice lacking Akt2 and Akt3 are viable but display impaired glucose homeostasis and growth deficiencies. Mol Cell Biol. 2006; 26(21):8042-51.
52. Hung C M, Lombardo P S, Malik N, Brun S N, Hellberg K, Van Nostrand J L, Garcia D, Baumgart J, Diffenderfer K, Asara J M, Shaw R J. AMPK/ULK1-mediated phosphorylation of Parkin ACT domain mediates an early step in mitophagy. Sci Adv. 2021; 7(15).
53. Emery J M, Ortiz R M. Mitofusin 2: A Link Between Mitochondrial Function and Substrate Metabolism? Mitochondrion. 2021.
54. Kawalec M, Boratynska-Jasinska A, Beresewicz M, Dymkowska D, Zablocki K, Zablocka B. Mitofusin 2 Deficiency Affects Energy Metabolism and Mitochondrial Biogenesis in MEF Cells. PLoS One. 2015; 10(7):e0134162.
55. Xue R, Meng Q, Lu D, Liu X, Wang Y, Hao J. Mitofusin2 Induces Cell Autophagy of Pancreatic Cancer through Inhibiting the PI3K/Akt/mTOR Signaling Pathway. Oxid Med Cell Longev. 2018; 2018:2798070.
56. de la Cruz Lopez K G, Toledo Guzman M E, Sanchez E O, Garcia Carranca A. mTORC1 as a Regulator of Mitochondrial Functions and a Therapeutic Target in Cancer. Front Oncol. 2019; 9:1373.
57. Chen Z, Zhang J, Dong C, Li D, Yin Y, Yu W, Chen Y. TNFAIP8 regulates gastric cancer growth via mTOR-Akt-ULK1 pathway and autophagy signals. J Cell Mol Med. 2021; 25(7):3361-70.
58. Shiratsuchi H, Basson M D. Akt2, but not Akt1 or Akt3 mediates pressure-stimulated serum-opsonized latex bead phagocytosis through activating mTOR and p70 S6 kinase. J Cell Biochem. 2007; 102(2):353-67.
59. Cilenti L, Ambivero C T, Ward N, Alnemri E S, Germain D, Zervos A S. Inactivation of Omi/HtrA2 protease leads to the deregulation of mitochondrial Mulan E3 ubiquitin ligase and increased mitophagy. Biochim Biophys Acta. 2014; 1843(7):1295-307.
60. Ambivero C T, Cilenti L, Main S, Zervos A S. Mulan E3 ubiquitin ligase interacts with multiple E2 conjugating enzymes and participates in mitophagy by recruiting GABARAP. Cell Signal. 2014; 26(12):2921-9.
61. Bandau S, Knebel A, Gage Z O, Wood N T, Alexandru G. UBXN7 docks on neddylated cullin complexes using its UIM motif and causes HIF1alpha accumulation. BMC Biol. 2012; 10:36.
62. Clifford S C, Astuti D, Hooper L, Maxwell P H, Ratcliffe P J, Maher E R. The pVHL-associated SCF ubiquitin ligase complex: molecular genetic analysis of elongin B and C, Rbx1 and HIF-1alpha in renal cell carcinoma. Oncogene. 2001; 20(36):5067-74.
63. Hung Y P, Teragawa C, Kosaisawe N, Gillies T E, Pargett M, Minguet M, Distor K, Rocha-Gregg B L, Coloff J L, Keibler M A, Stephanopoulos G, Yellen G, Brugge J S, Albeck J G. Akt regulation of glycolysis mediates bioenergetic stability in epithelial cells. Elife. 2017; 6.
64. Chen M, Ji C, Yang Q, Gao S, Peng Y, Li Z, Gao X, Li Y, Jiang N, Zhang Y, Bian X, Chen C, Zhang K, Sanchis D, Yan F, Ye J. AKT2 regulates development and metabolic homeostasis via AMPK-dependent pathway in skeletal muscle. Clin Sci (Lond). 2020; 134(17):2381-98.
65. Pedersen B A, Wang W, Taylor J F, Khattab O S, Chen Y H, Edwards R A, Yazdi P G, Wang P H. Hepatic proteomic analysis revealed altered metabolic pathways in insulin resistant Akt1(+/−)/Akt2(−/−) mice. Metabolism. 2015; 64(12):1694-703.
66. Kondapaka S B, Singh S S, Dasmahapatra G P, Sausville E A, Roy K K. Perifosine, a novel alkylphospholipid, inhibits protein kinase B activation 2003; 2(11):1093-103.
67. Kong D, Park E J, Stephen A G, Calvani M, Cardellina J H, Monks A, Fisher R J, Shoemaker R H, Melillo G. Echinomycin, a small-molecule inhibitor of hypoxia-inducible factor-1 DNA-binding activity. Cancer Res. 2005; 65(19):9047-55.
68. Adamovich Y, Ladeuix B, Sobel J, Manella G, Neufeld-Cohen A, Assadi M H, Golik M, Kuperman Y, Tarasiuk A, Koeners M P, Asher G. Oxygen and Carbon Dioxide Rhythms Are Circadian Clock Controlled and Differentially Directed by Behavioral Signals. Cell Metab. 2019; 29(5):1092-103 e3.
69. Corrigan J K, Ramachandran D, He Y, Palmer C J, Jurczak M J, Chen R, Li B, Friedline R H, Kim J K, Ramsey J J, Lantier L, McGuinness O P, Mouse Metabolic Phenotyping Center Energy Balance Working G, Banks A S. A big-data approach to understanding metabolic rate and response to obesity in laboratory mice. Elife. 2020; 9.
70. Lancaster G I, Henstridge D C. Body Composition and Metabolic Caging Analysis in High Fat Fed Mice. J Vis Exp. 2018(135).
71. Malloy C R, Jones J G, Jeffrey F M, Jessen M E, Sherry A D. Contribution of various substrates to total citric acid cycle flux and anaplerosis as determined by 13C isotopomer analysis and 02 consumption in the heart. MAGMA. 1996; 4(1):35-46.
72. Biswas C, Shah N, Muthu M, La P, Fernando A P, Sengupta S, Yang G, Dennery P A. Nuclear heme oxygenase-1 (H0-1) modulates subcellular distribution and activation of Nrf2, impacting metabolic and anti-oxidant defenses. J Biol Chem. 2014; 289(39):26882-94.
73. Hayashi M, Sakata M, Takeda T, Yamamoto T, Okamoto Y, Sawada K, Kimura A, Minekawa R, Tahara M, Tasaka K, Murata Y. Induction of glucose transporter 1 expression through hypoxia-inducible factor 1alpha under hypoxic conditions in trophoblast-derived cells. J Endocrinol. 2004; 183(1):145-54.
74. Jiang T, Harder B, Rojo de la Vega M, Wong P K, Chapman E, Zhang D D. p62 links autophagy and Nrf2 signaling Free Radic Biol Med. 2015; 88(Pt B):199-204.
75. Limonciel A, Jennings P. A review of the evidence that ochratoxin A is an Nrf2 inhibitor: implications for nephrotoxicity and renal carcinogenicity. Toxins (Basel). 2014; 6(1):371-9.
76. Fu C Y, Chen J, Lu X Y, Zheng M Z, Wang L L, Shen Y L, Chen Y Y. Dimethyl fumarate attenuates lipopolysaccharide-induced mitochondrial injury by activating Nrf2 pathway in cardiomyocytes. Life Sci. 2019; 235:116863.
77. Cilenti L, Soundarapandian M M, Kyriazis G A, Stratico V, Singh S, Gupta S, Bonventre J V, Alnemri E S, Zervos A S. Regulation of HAX-1 anti-apoptotic protein by Omi/HtrA2 protease during cell death. The Journal of biological chemistry. 2004; 279(48):50295-301.
78. Ebner M, Lucic I, Leonard T A, Yudushkin I. PI(3,4,5)P3 Engagement Restricts Akt Activity to Cellular Membranes. Mol Cell. 2017; 65(3):416-31 e6.
79. Li T Y, Sun Y, Liang Y, Liu Q, Shi Y, Zhang C S, Zhang C, Song L, Zhang P, Zhang X, Li X, Chen T, Huang H Y, He X, Wang Y, Wu Y Q, Chen S, Jiang M, Chen C, Xie C, Yang J Y, Lin Y, Zhao S, Ye Z, Lin S Y, Chiu D T, Lin SC. ULK1/2 Constitute a Bifurcate Node Controlling Glucose Metabolic Fluxes in Addition to Autophagy. Mol Cell. 2016; 62(3):359-70.
80. Ro S H, Jung C H, Hahn W S, Xu X, Kim Y M, Yun Y S, Park J M, Kim K H, Seo M, Ha T Y, Arriaga E A, Bernlohr D A, Kim D H. Distinct functions of Ulk1 and Ulk2 in the regulation of lipid metabolism in adipocytes. Autophagy. 2013; 9(12):2103-14.
81. Mandaviani K, Benador I Y, Su S, Gharakhanian R A, Stiles L, Trudeau K M, Cardamone M, Enriquez-Zarralanga V, Ritou E, Aprahamian T, Oliveira M F, Corkey B E, Perissi V, Liesa M, Shirihai O S. Mfn2 deletion in brown adipose tissue protects from insulin resistance and impairs thermogenesis. EMBO Rep. 2017; 18(7):1123-38.
82. Benador I Y, Veliova M, Liesa M, Shirihai O S. Mitochondria Bound to Lipid Droplets: Where Mitochondrial Dynamics Regulate Lipid Storage and Utilization. Cell Metab. 2019; 29(4):827-35.
83. Liesa M, Shirihai O S. Mitochondrial dynamics in the regulation of nutrient utilization and energy expenditure. Cell Metab. 2013; 17(4):491-506.
84. Li T, Han J, Jia L, Hu X, Chen L, Wang Y. PKM2 coordinates glycolysis with mitochondrial fusion and oxidative phosphorylation. Protein Cell. 2019; 10(8):583-94.
85. Boutant M, Kulkarni S S, Joffraud M, Ratajczak J, Valera-Alberni M, Combe R, Zorzano A, Canto C Mfn2 is critical for brown adipose tissue thermogenic function. EMBO J. 2017; 36(11):1543-58.
86. Mancini G, Pirruccio K, Yang X, Bluher M, Rodeheffer M, Horvath T L. Mitofusin 2 in Mature Adipocytes Controls Adiposity and Body Weight. Cell Rep. 2019; 27(2):648.
87. Bartolome A, Garcia-Aguilar A, Asahara S I, Kido Y, Guillen C, Pajvani U B, Benito M. MTORC1 Regulates both General Autophagy and Mitophagy Induction after Oxidative Phosphorylation Uncoupling. Mol Cell Biol. 2017; 37(23).
88. Luo Q Y, Huo P, Wang L L, Wu X H. The influencing mechanism of mTOR signal pathway mediated by mitofusin-2 in development of follicle. Eur Rev Med Pharmacol Sci. 2018; 22(8):2212-7.
89. Morita M, Prudent J, Basu K, Goyon V, Katsumura S, Hulea L, Pearl D, Siddiqui N, Strack S, McGuirk S, St-Pierre J, Larsson O, Topisirovic I, Vali H, McBride H M, Bergeron J J, Sonenberg N. mTOR Controls Mitochondrial Dynamics and Cell Survival via MTFP1. Mol Cell. 2017; 67(6):922-35 e5.
90. Wang X, Wu W K K, Gao J, Li Z, Dong B, Lin X, Li Y, Li Y, Gong J, Qi C, Peng Z, Yu J, Shen L. Autophagy inhibition enhances PD-L1 expression in gastric cancer. J Exp Clin Cancer Res. 2019; 38(1):140.
91. Egan D F, Chun M G, Vamos M, Zou H, Rong J, Miller C J, Lou H J, Raveendra-Panickar D, Yang C C, Sheffler D J, Teriete P, Asara J M, Turk B E, Cosford N D, Shaw R J. Small Molecule Inhibition of the Autophagy Kinase ULK1 and Identification of ULK1 Substrates. Mol Cell. 2015; 59(2):285-97.
92. Loffler A S, Alers S, Dieterle A M, Keppeler H, Franz-Wachtel M, Kundu M, Campbell D G, Wesselborg S, Alessi D R, Stork B. Ulk1-mediated phosphorylation of AMPK constitutes a negative regulatory feedback loop. Autophagy. 2011; 7(7):696-706.
93. Charni-Natan M, Goldstein I. Protocol for Primary Mouse Hepatocyte Isolation. STAR Protoc. 2020; 1(2):100086.
94. Kim Y M, Krantz S, Jambusaria A, Toth P T, Moon H G, Gunarathna I, Park G Y, Rehman J. Mitofusin-2 stabilizes adherens junctions and suppresses endothelial inflammation via modulation of beta-catenin signaling Nat Commun 2021; 12(1):2736.
95. Um J H, Kim Y Y, Finkel T, Yun J. Sensitive Measurement of Mitophagy by Flow Cytometry Using the pH-dependent Fluorescent Reporter mt-Keima. J Vis Exp. 2018(138).
96. Sun N, Malide D, Liu J, Rovira, II, Combs C A, Finkel T. A fluorescence-based imaging method to measure in vitro and in vivo mitophagy using mt-Keima. Nat Protoc. 2017; 12(8):1576-87.
97. Winsor N J, Killackey S A, Philpott D J, Girardin S E. An optimized procedure for quantitative analysis of mitophagy with the mtKeima system using flow cytometry. Biotechniques. 2020; 69(4):249-56.
98. Dagda R K, Chu C T. Mitochondrial quality control: insights on how Parkinson's disease related genes PINK1, parkin, and Omi/HtrA2 interact to maintain mitochondrial homeostasis. Journal of bioenergetics and biomembranes. 2009; 41(6):473-9.
99. Radke S, Chander H, Schafer P, Meiss G, Kruger R, Schulz J B, Germain D Mitochondrial protein quality control by the proteasome involves ubiquitination and the protease Omi. The Journal of biological chemistry. 2008; 283(19):12681-5.
100. Chaganti L K, Kuppili R R, Bose K. Intricate structural coordination and domain plasticity regulate activity of serine protease HtrA2. FASEB journal: official publication of the Federation of American Societies for Experimental Biology. 2013.
101. Kummari R, Dutta S, Patil S, Mudrale S P, Bose K. Elucidating the role of GRIM-19 as a substrate and allosteric activator of pro-apoptotic serine protease HtrA2. Biochem J. 2021; 478(6):1241-59.
102. Liu Q B, Liu L L, Lu Y M, Tao R R, Huang J Y, Han F, Lou Y J. The induction of reactive oxygen species and loss of mitochondrial Omi/HtrA2 is associated with S-nitrosoglutathione-induced apoptosis in human endothelial cells. Toxicology and applied pharmacology. 2010; 244(3):374-84.
103. Ruan L, Zhou C, Jin E, Kucharavy A, Zhang Y, Wen Z, Florens L, Li R. Cytosolic proteostasis through importing of misfolded proteins into mitochondria. Nature. 2017; 543(7645):443-6.
104. Hu H, Nan J, Sun Y, Zhu D, Xiao C, Wang Y, Zhu L, Wu Y, Zhao J, Wu R, Chen J, Yu H, Hu X, Zhu W, Wang J. Electron leak from NDUFA13 within mitochondrial complex I attenuates ischemia-reperfusion injury via dimerized STAT3. Proc Natl Acad Sci USA. 2017; 114(45):11908-13.
105. Cilenti L, Kyriazis G A, Soundarapandian M M, Stratico V, Yerkes A, Park K M, Sheridan A M, Alnemri E S, Bonventre J V, Zervos A S. Omi/HtrA2 protease mediates cisplatin-induced cell death in renal cells. American journal of physiology Renal physiology. 2005; 288(2):F371-9.
106. Cilenti L, Lee Y, Hess S, Srinivasula S, Park K M, Junqueira D, Davis H, Bonventre J V, Alnemri E S, Zervos A S. Characterization of a novel and specific inhibitor for the pro-apoptotic protease Omi/HtrA2. The Journal of biological chemistry. 2003; 278(13):11489-94.
107. Lee J, An S, Jung J H, Kim K, Kim J Y, An I S, Bae S. MUL1 E3 ligase regulates the antitumor effects of metformin in chemoresistant ovarian cancer cells via AKT degradation. Int J Oncol. 2019; 54(5):1833-42.
108. Garcia-Eguren G, Sala-Vila A, Giro O, Vega-Beyhart A, Hanzu F A. Long-term hypercortisolism induces lipogenesis promoting palmitic acid accumulation and inflammation in visceral adipose tissue compared with HFD-induced obesity. Am J Physiol Endocrinol Metab. 2020; 318(6):E995-E1003.
109. Li Z, Oh D Y, Nakamura K, Thiele C J. Perifosine-induced inhibition of Akt attenuates brain-derived neurotrophic factor/TrkB-induced chemoresistance in neuroblastoma in vivo. Cancer. 2011; 117(23):5412-22.
110. Li Z, Tan F, Liewehr D J, Steinberg S M, Thiele C J. In vitro and in vivo inhibition of neuroblastoma tumor cell growth by AKT inhibitor perifosine. J Natl Cancer Inst. 2010; 102(11):758-70.
111. Wang Y, Liu Y, Tang F, Bernot K M, Schore R, Marcucci G, Caligiuri M A, Zheng P, Liu Y. Echinomycin protects mice against relapsed acute myeloid leukemia without adverse effect on hematopoietic stem cells. Blood. 2014; 124(7):1127-35.
112. Malloy C R, Jones J G, Jeffrey F M, Jessen M E, Sherry A D. Contribution of various substrates to total citric acid cycle flux and anaplerosis as determined by&lt;sup&gt;13&lt;/sup&gt;C isotopomer analysis and O&lt;sub&gt;2&lt;/sub&gt; consumption in the heart. Magnetic Resonance Materials in Physics, Biology and Medicine. 1996; 4(1):35-46.
113. Whyte R K, Bayley H, Schwarcz H. The measurement of whole body water by H218O dilution in newborn pigs. The American journal of clinical nutrition. 1985; 41(4):801-9.
114. Raman A, Blanc S, Adams A, Schoeller DA. Validation of deuterium-labeled fatty acids for the measurement of dietary fat oxidation during physical activity. Journal of lipid research. 2004; 45(12):2339-44.
115. Votruba S, Zeddun S, Schoeller D. Validation of deuterium labeled fatty acids for the measurement of dietary fat oxidation: a method for measuring fat-oxidation in free-living subjects. International journal of obesity. 2001; 25(8):1240-5.
116. Sidossis L, Coggan A, Gastaldelli A, Wolfe R. A new correction factor for use in tracer estimations of plasma fatty acid oxidation. American Journal of Physiology-Endocrinology And Metabolism. 1995; 269(4):E649-E56.
117. Jones J G, Perdigoto R, Rodrigues T B, Geraldes C F G C. Quantitation of absolute 2H enrichment of plasma glucose by 2H NMR analysis of its monoacetone derivative. Magnetic Resonance in Medicine. 2002; 48(3):535-9.
118. Beysen C, Murphy E J, McLaughlin T, Riiff T, Lamendola C, Turner H C, Awada M, Turner S M, Reaven G, Hellerstein M K. Whole-body glycolysis measured by the deuterated-glucose disposal test correlates highly with insulin resistance in vivo. Diabetes care. 2007; 30(5):1143-9.
119. Mahar R, Donabedian P, Merritt M E. HDO production from [²H₇] glucose Quantitatively Identifies Warburg Metabolism. Scientific Reports (Nature Publisher Group). 2020; 10(1).
120. Mahar R, Zeng H, Giacalone A, Ragavan M, Mareci T H, Merritt M. HDO Imaging of the Rat Brain Following Metabolism of [²H₇] glucose. Mag Res Med. 2021; in Press. doi: http://dx.doi.org/10.1002/mrm.28700
121. Mahar R, Chang M, Merritt M. Measuring NQO1 Bioactivation Using [²H₇]Glucose. Cancers. 2021; 13(16):4165-83.
122. Bal N C, Maurya S K, Sopariwala D H, Sahoo S K, Gupta S C, Shaikh S A, Pant M, Rowland L A, Bombardier E, Goonasekera S A, Tupling A R, Molkentin J D, Periasamy M. Sarcolipin is a newly identified regulator of muscle-based thermogenesis in mammals. Nat Med. 2012; 18(10):1575-9.
123. Bal N C, Singh S, Reis F C G, Maurya S K, Pani S, Rowland L A, Periasamy M. Both brown adipose tissue and skeletal muscle thermogenesis processes are activated during mild to severe cold adaptation in mice. J Biol Chem. 2017; 292(40):16616-25.
124. Rowland L A, Bal N C, Kozak L P, Periasamy M. Uncoupling Protein 1 and Sarcolipin Are Required to Maintain Optimal Thermogenesis, and Loss of Both Systems Compromises Survival of Mice under Cold Stress. J Biol Chem. 2015; 290(19):12282-9.
125. Maurya S K, Bal N C, Sopariwala D H, Pant M, Rowland L A, Shaikh S A, Periasamy M. Sarcolipin Is a Key Determinant of the Basal Metabolic Rate, and Its Overexpression Enhances Energy Expenditure and Resistance against Diet-induced Obesity. J Biol Chem. 2015; 290(17):10840-9.
126. Hasan N, Nagata N, Morishige J I, Islam M T, Jing Z, Harada K I, Mieda M, Ono M, Fujiwara H, Daikoku T, Fujiwara T, Maida Y, Ota T, Shimba S, Kaneko S, Fujimura A, Ando H. Brown adipocyte-specific knockout of Bmal1 causes mild but significant thermogenesis impairment in mice. Mol Metab. 2021; 49:101202.
127. Votruba S B, Atkinson R L, Schoeller D A. Prior exercise increases dietary oleate, but not palmitate oxidation. Obesity research. 2003; 11(12):1509-18.
128. Alger, J. R., Minhajuddin, A., Dean Sherry, A., and Malloy, C.R. (2021). Analysis of steady-state carbon tracer experiments using akaike information criteria. Metabolomics 17, 61.
129. Annesley, S. J., and Fisher, P.R. (2019). Mitochondria in Health and Disease. Cells 8. doi: 10.3390/cells8070680.
130. Chen, C., Mahar, R., Merritt, M. E., Denlinger, D. L., and Hahn, D. A. (2021). ROS and hypoxia signaling regulate periodic metabolic arousal during insect dormancy to coordinate glucose, amino acid, and lipid metabolism. Proc Natl Acad Sci USA 118.doi: 10.1073/pnas.2017603118.
131. De Padua, M. C., Delodi, G., Vucetic, M., Durivault, J., Vial, V., Bayer, P., Noleto, G. R., Mazure, N. M., Zdralevic, M., and Pouyssegur, J. (2017). Disrupting glucose-6-phosphate isomerase fully suppresses the “Warburg effect” and activates OXPHOS with minimal impact on tumor growth except in hypoxia. Oncotarget 8, 87623-87637.doi: 10.18632/oncotarget.21007.
132. Escobar-Henriques, M., and Joaquim, M. (2019). Mitofusins: Disease Gatekeepers and Hubs in Mitochondrial Quality Control by E3 Ligases. Front Physiol 10, 517.doi: 10.3389/fphys.2019.00517.
133. Faubert, B., Solmonson, A., and Deberardinis, R. J. (2020). Metabolic reprogramming and cancer progression. Science 368.doi: 10.1126/science.aaw5473.
134. Fuhrmann, D. C., and Brune, B. (2017). Mitochondrial composition and function under the control of hypoxia. Redox Biol 12, 208-215.doi: 10.1016/j.redox.2017.02.012.
135. Glancy, B., Kim, Y., Katti, P., and Willingham, T. B. (2020). The Functional Impact of Mitochondrial Structure Across Subcellular Scales. Front Physiol 11, 541040.
136. Glickman, M. H., and Ciechanover, A. (2002). The ubiquitin-proteasome proteolytic pathway: destruction for the sake of construction. Physiological reviews 82, 373-428.
137. Gonzalez, E., and Mcgraw, T. E. (2009). The Akt kinases: isoform specificity in metabolism and cancer. Cell Cycle 8, 2502-2508.doi: 10.4161/cc.8.16.9335.
138. Herst, P. M., Rowe, M. R., Carson, G. M., and Berridge, M. V. (2017). Functional Mitochondria in Health and Disease. Front Endocrinol (Lausanne) 8, 296.doi: 10.3389/fendo.2017.00296.
139. Jin, E. S., Moreno, K. X., Wang, J. X., Fidelino, L., Merritt, M. E., Sherry, A. D., and Malloy, C. R. (2016). Metabolism of hyperpolarized [1-(13)C]pyruvate through alternate pathways in rat liver. NMR Biomed 29, 466-474.doi: 10.1002/nbm.3479.
140. Kung, A. L., Zabludoff, S. D., France, D. S., Freedman, S. J., Tanner, E. A., Vieira, A., Cornell-Kennon, S., Lee, J., Wang, B., Wang, J., Memmert, K., Naegeli, H. U., Petersen, F., Eck, M. J., Bair, K. W., Wood, A. W., and Livingston, D. M. (2004) Small molecule blockade of transcriptional coactivation of the hypoxia-inducible factor pathway. Cancer Cell 6, 33-43.
141. Kuo, Y. C., Huang, K. Y., Yang, C. H., Yang, Y. S., Lee, W. Y., and Chiang, C. W. (2008). Regulation of phosphorylation of Thr-308 of Akt, cell proliferation, and survival by the B55alpha regulatory subunit targeting of the protein phosphatase 2A holoenzyme to Akt. J Biol Chem 283, 1882-1892.doi: 10.1074/jbc.M709585200.
142. Le Grand, M., Berges, R., Pasquier, E., Montero, M. P., Borge, L., Carrier, A., Vasseur, S., Bourgarel, V., Buric, D., Andre, N., Braguer, D., and Cane, M. (2017). Akt targeting as a strategy to boost chemotherapy efficacy in non-small cell lung cancer through metabolism suppression. Sci Rep 7, 45136.doi: 10.1038/srep45136.
143. Le Grand, M., Rovini, A., Bourgarel-Rey, V., Honore, S., Bastonero, S., Braguer, D., and Cane, M. (2014). ROS-mediated EB1 phosphorylation through Akt/GSK3beta pathway: implication in cancer cell response to microtubule-targeting agents. Oncotarget 5, 3408-3423.
144. Livnat-Levanon, N., and Glickman, M. H. (2011). Ubiquitin-proteasome system and mitochondria—reciprocity. Biochimica et biophysica acta 1809, 80-87.
145. Lu, H., Forbes, R. A., and Verma, A. (2002). Hypoxia-inducible factor 1 activation by aerobic glycolysis implicates the Warburg effect in carcinogenesis. J Biol Chem 277, 23111-23115.
146. Mcelroy, G. S., and Chandel, N. S. (2017). Mitochondria control acute and chronic responses to hypoxia. Exp Cell Res 356, 217-222.doi: 10.1016/j.yexcr.2017.03.034.
147. Miccheli, A. T., Miccheli, A., Di Clemente, R., Valerio, M., Coluccia, P., Bizzarri, M., and Conti, F. (2006). NMR-based metabolic profiling of human hepatoma cells in relation to cell growth by culture media analysis. Biochim Biophys Acta 1760, 1723-1731.doi: 10.1016/j.bbagen.2006.09.009.
148. Mishra, P., and Chan, D. C. (2016). Metabolic regulation of mitochondrial dynamics. J Cell Biol 212, 379-387.doi: 10.1083/jcb.201511036.
149. Nemazanyy, I., Espeillac, C., Pende, M., and Panasyuk, G. (2013). Role of PI3K, mTOR and Akt2 signalling in hepatic tumorigenesis via the control of PKM2 expression. Biochem Soc Trans 41, 917-922.doi: 10.1042/BST20130034.
150. Park, S., Jeon, J. H., Min, B. K., Ha, C. M., Thoudam, T., Park, B. Y., and Lee, I. K. (2018). Role of the Pyruvate Dehydrogenase Complex in Metabolic Remodeling: Differential Pyruvate Dehydrogenase Complex Functions in Metabolism. Diabetes Metab J 42, 270-281.
151. Pavlides, S., Whitaker-Menezes, D., Castello-Cros, R., Flomenberg, N., Witkiewicz, A. K., Frank, P. G., Casimiro, M. C., Wang, C., Fortina, P., Addya, S., Pestell, R. G., Martinez-Outschoorn, U. E., Sotgia, F., and Lisanti, M. P. (2009). The reverse Warburg effect: aerobic glycolysis in cancer associated fibroblasts and the tumor stroma. Cell Cycle 8, 3984-4001.doi: 10.4161/cc.8.23.10238.
152. Peng, J., Ren, K. D., Yang, J., and Luo, X. J. (2016). Mitochondrial E3 ubiquitin ligase 1: A key enzyme in regulation of mitochondrial dynamics and functions. Mitochondrion 28, 49-53.
153. Purmal, C., Kucejova, B., Sherry, A. D., Burgess, S. C., Malloy, C. R., and Merritt, M. E. (2014). Propionate stimulates pyruvate oxidation in the presence of acetate. Am J Physiol Heart Circ Physiol 307, H1134-1141.doi: 10.1152/ajpheart.00407.2014.
154. Ramaswamy, V., Hooker, J. W., Withers, R. S., Nast, R. E., Brey, W. W., and Edison, A. S. (2013). Development of a (1)(3)C-optimized 1.5-mm high temperature superconducting NMR probe. J Magn Reson 235, 58-65.doi: 10.1016/j.jmr.2013.07.012.
155. Ran, F. A., Hsu, P. D., Wright, J., Agarwala, V., Scott, D. A., and Zhang, F. (2013). Genome engineering using the CRISPR-Cas9 system. Nat Protoc 8, 2281-2308.doi: 10.1038/nprot.2013.143.
156. Rossmann, M. P., Dubois, S. M., Agarwal, S., and Zon, L. I. (2021). Mitochondrial function in development and disease. Dis Model Mech 14.doi: 10.1242/dmm.048912.
157. Sakaguchi, A., and Kimura, W. (2021). Metabolic regulation of cardiac regeneration: roles of hypoxia, energy homeostasis, and mitochondrial dynamics. Curr Opin Genet Dev 70, 54-60.
158. Staab, A., Loeffler, J., Said, H. M., Diehlmann, D, Katzer, A., Beyer, M., Fleischer, M., Schwab, F., Baier, K., Einsele, H., Flentje, M., and Vordermark, D. (2007). Effects of HIF-1 inhibition by chetomin on hypoxia-related transcription and radiosensitivity in HT 1080 human fibrosarcoma cells. BMC Cancer 7, 213.doi: 10.1186/1471-2407-7-213.
159. Steffen, J., Vashisht, A. A., Wan, J., Jen, J. C., Claypool, S. M., Wohlschlegel, J. A., and Koehler, C. M. (2017). Rapid degradation of mutant SLC25A46 by the ubiquitin-proteasome system results in MFN1/2-mediated hyperfusion of mitochondria. Mol Biol Cell 28, 600-612
160. Weinberg, S. E., and Chandel, N. S. (2015). Targeting mitochondria metabolism for cancer therapy. Nat Chem Biol 11, 9-15.doi: 10.1038/nchembio.1712.
161. Yuan, M., Kremer, D. M., Huang, H., Breitkopf, S. B., Ben-Sahra, I., Manning, B. D., Lyssiotis, C. A., and Asara, J. M. (2019). Ex vivo and in vivo stable isotope labelling of central carbon metabolism and related pathways with analysis by LC-MS/MS. Nat Protoc 14, 313-330.
162. Zhdanov, A. V., Okkelman, I. A., Collins, F. W., Melgar, S., and Papkovsky, D. B. (2015). A novel effect of DMOG on cell metabolism: direct inhibition of mitochondrial function precedes HIF target gene expression. Biochim Biophys Acta 1847, 1254-1266.doi: 10.1016/j.bbabio.2015.06.016.
[163] Devin, A., Rigoulet, M., 2007. Mechanisms of mitochondrial response to variations in energy demand in eukaryotic cells. Am J Physiol Cell Physiol 292(1):C52-58.
[164] Yoboue, E. D., Mougeolle, A., Kaiser, L., Averet, N., Rigoulet, M., Devin, A., 2014. The role of mitochondrial biogenesis and ROS in the control of energy supply in proliferating cells. Biochim Biophys Acta 1837(7):1093-1098.
[165] Spinelli, J. B., Haigis, M. C., 2018. The multifaceted contributions of mitochondria to cellular metabolism. Nat Cell Biol 20(7):745-754.
[166] Jung, J. H., Bae, S., Lee, J. Y., Woo, S. R., Cha, H. J., Yoon, Y., et al., 2011. E3 ubiquitin ligaseHades negatively regulates the exonuclear function of p53. Cell death and differentiation 18(12):1865-1875.
[167] Zhang, B., Huang, J., Li, H. L., Liu, T., Wang, Y. Y., Waterman, P., et al., 2008. GIDE is a mitochondrial E3 ubiquitin ligase that induces apoptosis and slows growth. Cell research 18(9):900-910.
[168] Jenkins, K., Khoo, J. J., Sadler, A., Piganis, R., Wang, D., Borg, N. A., et al., 2013. Mitochondrially localised MUL1 is a novel modulator of antiviral signaling Immunology and cell biology 91(4):321-330.
[169] Puri, R., Cheng, X. T., Lin, M. Y., Huang, N., Sheng, Z. H., 2020. Defending stressed mitochondria: uncovering the role of MUL1 in suppressing neuronal mitophagy. Autophagy 16(1):176-178.
[170] Calle, X., Garrido-Moreno, V., Lopez-Gallardo, E., Norambuena-Soto, I., Martinez, D., Penaloza-Otarola, A., et al., 2022. Mitochondrial E3 ubiquitin ligase 1 (MUL1) as a novel therapeutic target for diseases associated with mitochondrial dysfunction. IUBMB Life 74(9):850-865.
[171] Doiron, K., Goyon, V., Coyaud, E., Rajapakse, S., Raught, B., McBride, H. M., 2017. The dynamic interacting landscape of MAPL reveals essential functions for SUMOylation in innate immunity. Sci Rep 7(1):107.
[172] Scorrano, L., Liu, D., 2009. The SUMO arena goes mitochondrial with MAPL. EMBO reports 10(7):694-696.
[173] Barry, R., John, S. W., Liccardi, G., Tenev, T., Jaco, I., Chen, C. H., et al., 2018. SUMOmediated regulation of NLRP3 modulates inflammasome activity. Nat Commun 9(1):3001.
[174] Cilenti, L., Mahar, R., Di Gregorio, J., Ambivero, C. T., Merritt, M. E., Zervos, A. S., 2022. Regulation of Metabolism by Mitochondrial MUL1 E3 Ubiquitin Ligase. Front Cell Dev Biol 10:904728.
[175] Faccio, L., Fusco, C., Chen, A., Martinotti, S., Bonventre, J. V., Zervos, A. S., 2000. Characterization of a novel human serine protease that has extensive homology to bacterial heat shock endoprotease HtrA and is regulated by kidney ischemia. The Journal of biological chemistry 275(4):2581-2588.
[176] Goyon, V., Besse-Patin, A., Zunino, R., Nguyen, M., Coyaud, E., Lee, J. M., et al., 2022. SUMOylation of ABCD3 restricts bile acid synthesis and regulates metabolic homeostasis. bioRxiv:2022.2003.2003.482848.
[177] Dobrzyn, P., Dobrzyn, A., Miyazaki, M., Ntambi, J. M., 2010. Loss of stearoyl-CoA desaturase 1 rescues cardiac function in obese leptin-deficient mice. J Lipid Res 51(8):2202-2210.
[178] Flowers, J. B., Rabaglia, M. E., Schueler, K. L., Flowers, M. T., Lan, H., Keller, M. P., et al., 2007. Loss of stearoyl-CoA desaturase-1 improves insulin sensitivity in lean mice but worsens diabetes in leptin-deficient obese mice. Diabetes 56(5):1228-1239.
[179] Liu, X., Miyazaki, M., Flowers, M. T., Sampath, H., Zhao, M., Chu, K., et al., 2010. Loss of Stearoyl-CoA desaturase-1 attenuates adipocyte inflammation: effects of adipocyte-derived oleate. Arterioscler Thromb Vasc Biol 30(1):31-38.
[180] Ntambi, J. M., Miyazaki, M., Stoehr, J. P., Lan, H., Kendziorski, C. M., Yandell, B. S., et al., 2002. Loss of stearoyl-CoA desaturase-1 function protects mice against adiposity. Proc Natl Acad Sci USA 99(17):11482-11486.
[181] Dobrzyn, P., Sampath, H., Dobrzyn, A., Miyazaki, M., Ntambi, J. M., 2008. Loss of stearoyl-CoA desaturase 1 inhibits fatty acid oxidation and increases glucose utilization in the heart. Am J Physiol Endocrinol Metab 294(2):E357-364.
[182] Johnson, D. H., Narayan, S., Wilson, D. L., Flask, C. A., 2012. Body composition analysis of obesity and hepatic steatosis in mice by relaxation compensated fat fraction (RCFF) MRI. J Magn Reson Imaging 35(4):837-843.
[183] Mehlem, A., Hagberg, C. E., Muhl, L., Eriksson, U., Falkevall, A., 2013. Imaging of neutral lipids by oil red O for analyzing the metabolic status in health and disease. Nat Protoc 8(6):1149-1154.
[184] Mina, A. I., LeClair, R. A., LeClair, K. B., Cohen, D. E., Lantier, L., Banks, A. S., 2018. CalR: A Web-Based Analysis Tool for Indirect calorimetry Experiments. Cell Metab 28(4):656-666 e651.
[185] John, L. M., Petersen, N., Gerstenberg, M. K., Torz, L., Pedersen, K., Christoffersen, B. O., et al., 2022. Housing-temperature reveals energy intake counter-balances energy expenditure in normal-weight, but not diet-induced obese, male mice. Commun Biol 5(1):946.
[186] Sadler, D. G., Treas, L., Sikes, J. D., Porter, C., 2022. A modest change in housing temperature alters whole body energy expenditure and adipocyte thermogenic capacity in 55 mice. Am J Physiol Endocrinol Metab 323(6):E517-E528.
[187] Koelmel, J. P., Kroeger, N. M., Ulmer, C. Z., Bowden, J. A., Patterson, R. E., Cochran, J. A., et al., 2017. LipidMatch: an automated workflow for rule-based lipid identification using untargeted high-resolution tandem mass spectrometry data. BMC Bioinformatics 18(1):331.
[188] Chong, J., Wishart, D. S., Xia, J., 2019. Using MetaboAnalyst 4.0 for Comprehensive and Integrative Metabolomics Data Analysis. Curr Protoc Bioinformatics 68(1):e86.
[189] Molenaar, M. R., Jeucken, A., Wassenaar, T. A., van de Lest, C. H. A., Brouwers, J. F., Helms, J. B., 2019. LION/web: a web-based ontology enrichment tool for lipidomic data analysis. Gigascience 8(6).
[190] Chen, Y., Lun, A. T., Smyth, G. K., 2016. From reads to genes to pathways: differential expression analysis of RNA-Seq experiments using Rsubread and the edgeR quasilikelihood pipeline. F1000Res 5:1438.
[191] Tu, C., Xiong, H., Hu, Y., Wang, W., Mei, G., Wang, H., et al., 2020. Cardiolipin Synthase 1 Ameliorates NASH Through Activating Transcription Factor 3 Transcriptional Inactivation. Hepatology 72(6):1949-1967.
[192] Joo, M., Sadikot, R. T., 2012. PGD synthase and PGD2 in immune response. Mediators Inflamm 2012:503128.
[193] Wu, X., Zheng, H., Yang, R., Luan, X., Zhang, L., Jin, Q., et al., 2019. Mouse trefoil factor 3 ameliorated high-fat-diet-induced hepatic steatosis via increasing peroxisome proliferator activated receptor-alpha-mediated fatty acid oxidation. Am J Physiol Endocrinol Metab 317(3):E436-E445.
[194] Yang, Y., Lin, Z., Lin, Q., Bei, W., Guo, J., 2022. Pathological and therapeutic roles of bioactive peptide trefoil factor 3 in diverse diseases: recent progress and perspective. Cell Death Dis 13(1):62.
[195] Ralston, J. C., Metherel, A. H., Stark, K. D., Mutch, D. M., 2016. SCD1 mediates the influence of exogenous saturated and monounsaturated fatty acids in adipocytes: Effects on cellular stress, inflammatory markers and fatty acid elongation. J Nutr Biochem 27:241-248.
[196] Weiss-Hersh, K., Garcia, A. L., Marosvolgyi, T., Szklenar, M., Decsi, T., Ruhl, R., 2020. Saturated and monounsaturated fatty acids in membranes are determined by the gene expression of their metabolizing enzymes SCD1 and ELOVL6 regulated by the intake of dietary fat. Eur J Nutr 59(6):2759-2769.
[197] Piccinin, E., Cariello, M., De Santis, S., Ducheix, S., Sabba, C., Ntambi, J. M., et al., 2019. Role of Oleic Acid in the Gut-Liver Axis: From Diet to the Regulation of Its Synthesis via Stearoyl-CoA Desaturase 1 (SCD1). Nutrients 11(10).
[198] Dragos, S. M., Bergeron, K. F., Desmarais, F., Suitor, K., Wright, D. C., Mounier, C., et al., 2017. Reduced SCD1 activity alters markers of fatty acid reesterification, glyceroneogenesis, and lipolysis in murine white adipose tissue and 3T3-L1 adipocytes. Am J Physiol Cell Physiol 313 (3):C295-C304.
[199] Kim, S. Y., Kim, H. J., Kim, H. J., Kim, D. H., Han, J. H., Byeon, H. K., et al., 2018. HSPA5 negatively regulates lysosomal activity through ubiquitination of MUL1 in head and neck cancer. Autophagy 14(3):385-403.
[200] Man, W. C., Miyazaki, M., Chu, K., Ntambi, J. M., 2006. Membrane topology of mouse stearoyl-CoA desaturase 1. J Biol Chem 281(2):1251-1260.
[201] Liu, J., Cinar, R., Xiong, K., Godlewski, G., Jourdan, T., Lin, Y., et al., 2013. Monounsaturated fatty acids generated via stearoyl CoA desaturase-1 are endogenous inhibitors of fatty acid amide hydrolase. Proc Natl Acad Sci USA 110(47):18832-18837.
[202] Dobrzyn, P., Dobrzyn, A., Miyazaki, M., Cohen, P., Asilmaz, E., Hardie, D. G., et al., 2004. Stearoyl-CoA desaturase 1 deficiency increases fatty acid oxidation by activating AMPactivated protein kinase in liver. Proc Natl Acad Sci USA 101(17):6409-6414.
[203] Bednarski, T., Olichwier, A., Opasinska, A., Pyrkowska, A., Gan, A. M., Ntambi, J. M., et al., 2016. Stearoyl-CoA desaturase 1 deficiency reduces lipid accumulation in the heart by activating lipolysis independently of peroxisome proliferator-activated receptor alpha. Biochim Biophys Acta 1861(12 Pt A):2029-2037.
[204] Kurikawa, N., Takagi, T., Wakimoto, S., Uto, Y., Terashima, H., Kono, K., et al., 2013. A novel inhibitor of stearoyl-CoA desaturase-1 attenuates hepatic lipid accumulation, liver injury and inflammation in model of nonalcoholic steatohepatitis. Biol Pharm Bull 36(2):259-267.
[205] A M, A. L., Syed, D. N., Ntambi, J. M., 2017. Insights into Stearoyl-CoA Desaturase-1 Regulation of Systemic Metabolism. Trends Endocrinol Metab 28(12):831-842.
[206] Kim, Y. C., Ntambi, J. M., 1999. Regulation of stearoyl-CoA desaturase genes: role in cellular metabolism and preadipocyte differentiation. Biochem Biophys Res Commun 266(1):1-4.
[207] Lounis, M. A., Bergeron, K. F., Burhans, M. S., Ntambi, J. M., Mounier, C., 2017. Oleate activates SREBP-1 signaling activity in SCD1-deficient hepatocytes. Am J Physiol Endocrinol Metab 313(6):E710-E720.
[208] Chu, K., Miyazaki, M., Man, W. C., Ntambi, J. M., 2006. Stearoyl-coenzyme A desaturase 1 deficiency protects against hypertriglyceridemia and increases plasma high-density lipoprotein cholesterol induced by liver X receptor activation. Mol Cell Biol 26(18):6786-6798.
[209] Puri, R., Cheng, X. T., Lin, M. Y., Huang, N., Sheng, Z. H., 2019. Mul1 restrains Parkinmediated mitophagy in mature neurons by maintaining ER-mitochondrial contacts. Nat Commun 10(1):3645.
[210] Rojansky, R., Cha, M. Y., Chan, D. C., 2016. Elimination of paternal mitochondria in mouse embryos occurs through autophagic degradation dependent on PARKIN and MUL1. Elife 5.
[211] Igarashi, R., Yamashita, S. I., Yamashita, T., Inoue, K., Fukuda, T., Fukuchi, T., et al., 2020. Gemcitabine induces Parkin-independent mitophagy through mitochondrial-resident E3 ligase MUL1-mediated stabilization of PINK1. Sci Rep 10(1):1465.
[212] Cui, C., Chen, S., Qiao, J., Qing, L., Wang, L., He, T., et al., 2018. PINK1-Parkin alleviates metabolic stress induced by obesity in adipose tissue and in 3T3-L1 preadipocytes. Biochem Biophys Res Commun 498(3):445-452.
[213] Moore, T. M., Cheng, L., Wolf, D. M., Ngo, J., Segawa, M., Zhu, X., et al., 2022. Parkin regulates adiposity by coordinating mitophagy with mitochondrial biogenesis in white adipocytes. Nat Commun 13(1):6661.

What is claimed is:

1. A gene expression signature indicative of inactivation of mitochondrial MUL1 E3 ubiquitin ligase, the gene expression signature exhibiting upregulation of trefoil factor 3 (TFF3), asparagine synthetase (ASNS), and cytochrome P450 2A4 (CYP2A4) genes and downregulation of stearoyl-CoA desaturase 1 (SCDI) gene.

2. A method for inhibiting lipogenesis in cells comprising:
providing cells;
inactivating mitochondrial MUL1 E3 ubiquitin ligase (MUL1) in the cells;
treating a portion of the cells with a simulator of a high fat diet for a pre-determined period of time;
using a remaining portion of the cells as untreated control cells;
applying a stain for identification of lipid droplets to the treated cells and untreated control cells;
identifying lipid droplets in the treated cells and in the untreated control cells using a stain capable of identification of lipids;
visualizing lipid droplets identified in the treated cells and in the untreated control cells using an optical instrument; and
comparing an amount of stained lipid droplets visualized in the treated cells to an amount of stained lipid droplets visualized in the untreated control cells, wherein a decrease of stained lipid droplets in the treated cells indicates inhibition of lipogenesis in the treated cells.

3. The method according to claim 2, wherein providing cells includes providing at least one of human cells and animal cells.

4. The method according to claim 2, wherein providing cells includes providing human liver cells.

5. The method according to claim 2, wherein inactivating Mul1 includes inactivating Mul1 using CRISPR-Cas9.

6. The method according to claim 2, wherein treating a portion of the cells with a simulator of a high fat diet for a pre-determined period of time includes treating a portion of the cells with oleic acid for 24 hours.

7. A method for protecting a subject against obesity induced by a high fat diet (HFD) by inactivation of mitochondrial MUL1 E3 ubiquitin ligase (MUL1), the method comprising:
providing an inactivator of MUL1; and
administering the inactivator of MUL1 to the subject, thereby inactivating MUL1 and protecting the subject against obesity induced by a high fat diet (HFD).

8. The method according to claim 7, wherein the subject is at least one of a human and an animal.

9. The method according to claim 7, wherein the inactivator of MUL1 is an inhibitor of MUL1.


SEQUENCE LISTING (2 sequences)

SEQ ID NO: 1 - DNA, 20 bp, synthetic construct
  source 1..20: mol_type = other DNA
gaccccatggactacaagtg

SEQ ID NO: 2 - DNA, 20 bp, synthetic construct
  source 1..20: mol_type = other DNA
gccgccgtcatggagagcgg